(19) United States
(12) Patent Application Publication
(10) Pub. No.: US 2026/0207083 A1
(43) Pub. Date: Jul. 23, 2026

(54) PHYSIOLOGICAL MONITORING APPARATUS AND METHODS

(71) Applicant: Amptron Medical, Inc., Carlsbad, CA (US)

(72) Inventors: Nirav Ashokbhai Patel, Carlsbad, CA (US); Nancy F. Dong, San Marcos, CA (US)

(21) Appl. No.: 19/566,672

(22) Filed: Mar. 13, 2026

Related U.S. Application Data
(63) Continuation-in-part of application No. PCT/US25/39507, filed on Jul. 28, 2025.
(60) Provisional application No. 63/664,007, filed on Jun. 25, 2024.

(51) Int. Cl. A61B 5/113; A61B 5/00; A61B 5/091; A61B 5/11

(52) U.S. Cl. CPC A61B 5/113; A61B 5/0002; A61B 5/091; A61B 5/1107; A61B 5/1126; A61B 5/4842; A61B 5/6823; A61B 5/6831; A61B 5/7267; A61B 5/7275; A61B 5/742; A61B 5/746; A61B 2562/164

(57) ABSTRACT

A real-time, AI-driven respiratory monitoring system is disclosed comprising a wearable diaphragm sensor configured to detect mechanomyographic signals, optionally with additional sensors including a photoplethysmographic (PPG) sensor, carbon dioxide (CO₂) sensor, and temperature sensor. A processor is configured to receive synchronized multi-sensor signals and execute machine learning algorithms trained to derive respiratory parameters such as respiratory rate, tidal volume, I:E ratio, minute volume, and rapid shallow breathing index. The system may classify respiratory abnormalities, generate adaptive alerts, and display trends through a graphical interface. Patient posture and symptom input may adjust model inference and alert thresholds. The system may operate in therapy-assist and post-therapy monitoring modes, support closed-loop ventilator control, and transmit time-stamped respiratory data and alerts to remote caregiver dashboards. A population-trained model may adapt alerts to patient-specific baselines and detect conditions including COPD exacerbation, neuromuscular decline, respiratory distress, post-surgical complications, and dialysis-related impairment.

### CROSS-REFERENCE TO RELATED APPLICATIONS

This application claims priority as a continuation of PCT/US25/39507, filed Jul. 28, 2025, which claims priority to U.S. Provisional Patent Application No. 63/664,007, filed Jun. 25, 2024; the contents of each of these applications are incorporated herein by reference in its entirety.

### FIELD

This disclosure relates to respiratory monitoring systems and methods. More specifically, this disclosure relates to non-invasive wearable systems that may incorporate, for example, multi-sensor signal acquisition and artificial intelligence (AI) algorithms for continuous, real-time monitoring, diagnosis, and management of respiratory health conditions in patients.

### BACKGROUND

The statements in this background section are provided to assist with understanding the present disclosure and the applications and uses of various example embodiments, and do not constitute prior art.

Accurate and continuous respiratory monitoring is increasingly important in clinical contexts such as chronic obstructive pulmonary disease (COPD), respiratory therapy, dialysis and post-surgical recovery. Conventional methods such as pulse oximetry or thoracic impedance may offer limited resolution or indirect measurements that can pose challenges for early detection or management of respiratory issues.

Accordingly, a need exists for improvements in physiological monitoring systems that may provide enhanced signal fidelity, greater parameter visibility, more adaptive alerting and response logic, broader patient context integration, and improved compatibility with existing therapy infrastructure. Conventional methods often fail to integrate multi-modal sensor data with AI for real-time adaptation, highlighting a gap this invention addresses.

### SUMMARY

Provided in various example embodiments are physiological monitoring apparatus and methods that may address one or more technical challenges associated with respiratory monitoring and management. The present improvements—are designed to support early detection, clinical decision-making, patient engagement, and long-term respiratory care in a variety of care environments. The invention introduces to a non-invasive, AI-driven system with multi-sensor integration for real-time respiratory monitoring and therapy adjustment.

The disclosed systems and methods provide concrete clinical benefits, including in various example embodiments improved early detection of respiratory deterioration (e.g., COPD exacerbation), increased safety between and during dialysis treatments (hemodialysis and peritoneal dialysis), post-surgical recovery, optimized ventilator weaning through RSBI monitoring, and reduced alarm fatigue through personalized threshold adaptation.

In various example embodiments, the disclosed respiratory monitoring system may include a combination of hardware, software, and signal processing components that work together to acquire, interpret, and act upon real-time physiological data. Example systems may comprise any or all of: (1) a wearable diaphragm sensor configured to detect respiratory effort via mechanomyographic, capacitive, or piezoelectric means; (2) optional secondary sensors including accelerometers, SpO₂ detectors, CO₂ sensors, and temperature sensors; (3) a base unit containing a processor, machine learning module, alert logic, graphical user interface (GUI), wireless transmitter, and cloud communication interface; (4) a GUI and clinician dashboard for real-time visualization and annotation; (5) real-time adaptive machine learning models trained to interpret multi-sensor input and generate patient-specific predictions, classifications, or threshold adaptations; and (6) closed-loop or semi-automated therapy interfaces, such as ventilator parameter control logic, configured to adjust settings based on signal-derived indices such as RSBI, MV, and I:E ratio. Systems according to various example embodiments may be used across inpatient, outpatient, or home care settings, and may support use cases including but not limited to COPD monitoring, post-operative respiratory assessment, dialysis mismatch detection, ventilator weaning optimization, and neuromuscular fatigue tracking.

These improvements are not abstract but rather improve the functioning of a respiratory monitoring system by, for example, reducing false positives and enhancing real-time accuracy through posture-aware adaptive thresholds and multi-sensor fusion.

For example, systems and methods are provided for real-time respiratory monitoring using a wearable device comprising multiple sensors including a flexible capacitive diaphragm sensor, a photoplethysmographic (PPG) sensor, a carbon dioxide (CO2) sensor, and a temperature sensor. These may be integrated with a processor configured to receive and synchronize time-varying physiological signals.

In various example embodiments, a system may include a machine learning engine trained to derive respiratory health indicators from non-invasive multi-sensor data. These include respiratory rate, tidal volume, respiratory mechanics, dyspnea detection, and rapid shallow breathing index, among others. In some example embodiments, a system may further include user interfaces for both clinicians and patients, an alert module for threshold deviations, and a ventilator controller for closed-loop therapy adjustment. The system supports wireless data transmission for remote monitoring. The system includes a non-invasive wearable sensor capturing mechanomyographic (MMG) data in three spatial dimensions via a flexible diaphragm sensor, and an accelerometer detecting patient motion, with a processor receiving time-varying signals and a machine learning module trained to derive parameters such as respiratory rate, inspiratory and expiratory phases, inspiratory-expiratory ratio, tidal volume, minute volume, respiratory mechanics, rapid shallow breathing index, cough detection, and dyspnea detection.

Patient posture data and self-reported symptoms may be used to adapt the inference model in real time. In some example embodiments, a cloud interface may support transmission of derived data to remote dashboards and electronic health records. In various example embodiments, a population-trained AI platform may allow further personalization of respiratory alerts based on normative datasets.

For example, provided in various example embodiments is a respiratory monitoring system comprising a wearable sensor configured to be positioned over a patient's diaphragm. The wearable sensor may include a flexible capacitive diaphragm sensor configured to detect mechanomyographic (MMG) data in three spatial dimensions, and an accelerometer configured to detect patient motion. A processor may be configured to receive time-varying signals from the diaphragm sensor and the accelerometer. A machine learning module, stored in memory and configured to be executed by the processor, may be trained to derive one or more respiratory parameters from the received signals. These parameters may include respiratory rate, inspiratory and expiratory phases, inspiratory-expiratory ratio, tidal volume, minute volume, respiratory mechanics, rapid shallow breathing index, cough detection, and dyspnea detection.

In some embodiments, the system may further include a GUI configured to display one or more of the respiratory parameters in real time, and an alert module configured to generate a notification when one or more of the derived respiratory parameters deviates from an adaptive or predefined threshold.

Also provided is a method of monitoring a patient's respiratory condition. The method may include detecting diaphragmatic motion using a wearable sensor positioned over the diaphragm, generating a time-varying signal representing three-dimensional diaphragm movement, processing the signal with a trained machine learning model to derive respiratory parameters such as respiratory rate and tidal volume, comparing at least one of the derived parameters to a threshold, and generating an alert when the threshold is exceeded.

In additional embodiments, a system is provided for monitoring respiratory parameters during and after therapy. This system may include a wearable respiratory sensor with a flexible capacitive element and an accelerometer, a processor configured to extract time-varying signals from the sensor, and a machine learning engine trained to derive parameters such as respiratory rate and minute volume. A user interface may display respiratory trends, and a therapy integration module may allow users to input therapy changes and correlate them with observed parameter changes to inform therapy adjustments. The system may operate in both a therapy-assist mode and a post-therapy monitoring mode.

In certain implementations, the wearable sensor may be secured to the patient using a non-elastic adjustable belt and buckle, and may include two or more stretchable electrodes separated by a dielectric polymer. The diaphragm sensor and accelerometer may be enclosed in a housing designed for patient comfort during extended use.

In various embodiments, the machine learning module may be a deep learning model with at least three hidden layers. The processor may be configured to derive both inspiratory and expiratory tidal volume. The machine learning module may also classify patient condition based on respiratory mechanics, such as identifying diaphragm atrophy, neuromuscular weakness, or restrictive lung disease. The model may be trained using data from both healthy individuals and unhealthy individuals with chronic conditions like obstructive pulmonary disease.

The GUI may be configured to display real-time active bar graphs showing current, minimum, maximum, and average values for selected respiratory parameters over a time window. The alert module may include a 360-degree visual indicator and may prioritize alerts based on a computed severity index involving multiple parameters.

In further embodiments, the therapy integration module may transmit control signals to a respiratory therapy device, based on respiratory trends or alert conditions. The user interface may also allow manual entry of therapy, medication, or patient status updates, which may be stored in association with time-stamped parameter records for later review. The processor may be configured to correlate changes in tidal volume and respiratory rate with clinical interventions to support therapy optimization.

In some implementations, the system may automatically switch between therapy-assist and monitoring modes based on the detected status of a connected respiratory support device. The post-therapy monitoring mode may allow for long-term analysis of stored respiratory data in local or cloud-based databases.

The system may include wireless communication and a cloud database interface, enabling continuous remote monitoring in home or outpatient settings and transmission of time-stamped data and alerts to remote caregivers. The machine learning module may analyze historical respiratory data to identify patterns indicating early-stage COPD exacerbation and compare current values to patient-specific baselines to detect clinical deterioration.

In additional embodiments, the system may include diaphragm sensor for multi-modal analysis, and the machine learning module may adapt classification thresholds based on an individual patient's history and physiological trends. The system may also classify or assist in identifying conditions such as airway inflammation due to infection, abdominal fluid retention, internal bleeding after surgery, and respiratory changes associated with delayed dialysis.

The wearable sensor may be configured for use during recovery from major surgeries such as liver, cardiac, or abdominal procedures, and may detect post-operative respiratory complications. The system may also be configured to generate visualizations that correlate manually entered treatment changes with respiratory data over time, and may produce three-dimensional surface plots of diaphragmatic activity based on tEAdi signal data.

Still further, provided in various example embodiments is a wearable patient monitoring system comprising: a thoracoabdominal muscle-mounted flexible capacitive sensor configured to measure mechanomyographic (MMG) data due to diaphragm activity; a photoplethysmographic (PPG) sensor configured to measure oxygen saturation (SpO₂); a carbon dioxide (CO₂) sensor and a temperature sensor; a processor configured to receive and synchronize the signals from these sensors and to generate synchronized multi-sensor signal sets; a machine learning model trained to derive respiratory health indicators such as respiratory rate, minute volume, and rapid shallow breathing index based on the synchronized signals; and a user interface configured to display these health indicators to a clinician in real time.

Also provided is a method of operating a respiratory monitoring system, comprising: acquiring patient posture data from a body-position sensor; receiving patient-reported symptom input through a GUI; collecting physiological signals from a diaphragm sensor and a PPG sensor; selecting one of a plurality of machine learning inference profiles based at least in part on the posture and symptom data; deriving a respiratory parameter such as respiratory rate or minute volume using the selected profile; and generating an alert when the derived parameter deviates from a context-specific threshold. In some embodiments, the selected profile may also dynamically adjust alert thresholds based on this contextual input. The patient posture data may include positions such as prone, supine, sitting, inclined, or walking.

Further provided in some embodiments is a cloud-integrated respiratory monitoring system comprising: a wearable respiratory sensor unit including a diaphragm sensor and a PPG sensor; a processor configured to derive respiratory parameters from these signals; a cloud interface module configured to transmit the derived parameters and associated time-stamped events to a remote server; a GUI hosted on the remote server and configured to display patient-specific respiratory trends along with clinician-entered treatment notes; and an alert engine configured to transmit prioritized alerts to remote caregivers based on deviations in respiratory trends.

In various embodiments, the machine learning model may be trained using datasets labeled with specific respiratory conditions, including COPD, respiratory fatigue, and ventilation-perfusion mismatch. The processor may be further configured to analyze the relationship between diaphragmatic effort and oxygen saturation over time to detect trends indicative of conditions such as neuromuscular decline or impaired gas exchange.

The system may be configured to calculate a severity score based on aggregated sensor trends to prioritize alerts. The GUI may include a real-time active dashboard showing multiple parameters such as SpO₂, respiratory rate, and minute volume. Cloud interface security may include HIPAA-compliant encryption and access controls. The interface may also support real-time updates across multiple patient profiles and allow clinician-entered treatment notes to be time-stamped and correlated with respiratory trends. The alert engine may rank notifications by severity, and the remote server may be configured to export data to electronic health record (EHR) systems.

In certain embodiments, the temperature sensor may be used to distinguish fever-induced hyperventilation from other causes. The CO₂ sensor may be used to track end-tidal CO₂ levels for ventilatory assessment. The system may apply real-time filtering to remove movement artifacts based on body position data.

The system may also support secure long-term storage of patient-reported symptom data and derived parameters, and enable longitudinal trend analysis. It may compare real-time parameters against patient-specific baselines to identify respiratory deviations associated with clinical events. Derived parameter trends may also be used to identify respiratory distress caused by airway inflammation, internal bleeding, or fluid overload.

In some configurations, the system may operate in either a therapy-assist mode, where it adjusts therapy settings based on sensor feedback, or a monitoring-only mode, where it passively tracks and displays data post-therapy. In some examples, the system may automatically dynamically select an inference model and adjust alert thresholds using a combination of real-time posture data, symptom input, and detected movement artifacts.

Also provided in various embodiments is a method of monitoring a patient following surgery, such as abdominal, liver, or cardiac procedures. This method may include positioning a wearable sensor over the patient's thoracoabdominal muscles (diaphragm), acquiring respiratory signals during recovery, deriving respiratory parameters such as respiratory rate, Tidal volume, IE ratio, RSBI and minute volume, detecting changes indicative of complications such as internal bleeding or fluid retention, and generating an alert or flagging the patient record for clinical review.

Yet further, provided in various example embodiments is a respiratory support system comprising: a wearable respiratory sensor configured to measure diaphragm movement and oxygen saturation; a processor configured to derive respiratory parameters including at least inhalation-exhalation phase, respiratory rate and minute volume from sensor signals; and a ventilator controller operatively coupled to a respiratory therapy device, the ventilator controller being configured to adjust one or more therapy parameters selected from pressure, flow rate, volume or oxygen concentration or to trigger a breath in response to changes in the derived respiratory parameters.

Also provided is a patient-managed respiratory monitoring system comprising: a non-invasive wearable diaphragm sensor configured to detect mechanomyographic activity; and a mobile device application configured to: receive sensor data wirelessly; display real-time respiratory trends to the patient; receive patient-reported inputs including symptoms or perceived distress; and transmit the data to a remote clinical dashboard.

Further provided is a respiratory monitoring system comprising: a diaphragm-mounted sensor configured to detect time-varying mechanomyographic (MMG) data corresponding to diaphragmatic effort; a photoplethysmographic (PPG) sensor configured to detect oxygen saturation (SpO₂); a processor configured to determine a time-correlated divergence between the diaphragmatic effort signal and the SpO₂ signal; an inference engine configured to identify a respiratory abnormality indicative of pre, post or during dialysis-dialysis fatigue or impaired gas exchange based on the divergence; and an alert module configured to generate a notification in response to detection of the respiratory abnormality.

Additionally provided is a respiratory analytics platform comprising: a server configured to receive multi-parameter respiratory data from a plurality of wearable monitoring systems; a machine learning model trained using population-level datasets labeled with respiratory outcomes to generate predictive thresholds; a patient-specific adaptation module configured to adjust alert sensitivity based on individualized deviation from the population-trained thresholds; and a notification engine configured to transmit alerts to clinicians when patient-specific adjusted thresholds are exceeded.

In some embodiments, the respiratory sensor may comprise both a diaphragm sensor and a PPG sensor. The ventilator controller may be configured to operate automatically without clinician input. The processor may be configured to calculate a rapid shallow breathing index (RSBI) and to adjust inspiratory pressure or volume accordingly.

In some embodiments, the mobile application may include voice-input fields for symptom reporting, may be configured to store historical respiratory data, and may transmit weekly summaries to a clinical provider. The wearable sensor may include a haptic feedback element configured to alert the patient.

The alert module may be configured to transmit a notification to a caregiver via a mobile application or web-based interface. The processor and sensors may be configured for continuous operation during an interdialytic period. The inference engine may comprise a rule-based threshold module and a neural network classifier configured to validate detection of the respiratory abnormality.

In some embodiments, the population-trained model may include clinical outcome labels such as hospitalization or COPD exacerbation. The patient-specific alert threshold may be configured to be recalculated weekly or more frequently based on trend data. The platform may be integrated with a hospital's electronic health record system for real-time decision support.

In safety-critical and other cases, the system may be configured to allow therapy adjustments to be confirmed by clinician override. The inference engine may be further configured to distinguish between diaphragm fatigue and central hypoventilation based on a time-correlated divergence between diaphragmatic effort signals and oxygen saturation signals.

In some embodiments, the machine learning engine may be calibrated using data from at least 100 patients. In other embodiments, the machine learning engine may be calibrated using data from at least 500 patients. In still other embodiments, the machine learning engine may be calibrated using data from at least 1,000 patients. The system may be configured to display alerts on both the mobile device and a remote clinical dashboard in synchrony. The mobile application may be configured to enable entry of therapy changes or medication updates and to display those alongside physiological parameter trends.

In further embodiments, the machine learning engine may compare patient respiratory patterns against condition-specific baselines to identify post-operative complications or overdue dialysis events.

The logic, models, and control operations described herein may be embodied as software stored on a non-transitory computer-readable medium and executed by one or more processors. In some embodiments, the code may be embedded within a local processor of the base unit or wearable device, or executed via remote cloud infrastructure. In various embodiments, the disclosed system may be implemented entirely in software, hosted on a cloud platform, and delivered to end users via mobile, browser-based, or API-accessible frontends without requiring any local base unit hardware.

The embodiments described in this summary are merely illustrative examples of various aspects that may be employed. These examples are not intended to limit the scope of the invention, which is defined solely by the claims. Various modifications, alternatives, and additional or different embodiments may be made without departing from the scope of the claims.

### BRIEF DESCRIPTION OF THE DRAWINGS

The accompanying drawings, which are incorporated in and constitute part of this specification, illustrate example embodiments and together with the description, explain various principles of the disclosed embodiments. For clarity, simplicity, and flexibility, not all elements, components, or specifications are defined in all drawings. Not all drawings corresponding to specific steps or embodiments of the system described herein are drawn to scale. Emphasis is instead placed on illustration of the nature, function, and product of the manufacturing method and devices described herein.

Embodiments described herein are exemplary and not restrictive. Embodiments will now be described, by way of examples, with reference to the accompanying drawings, in which:

FIG. 1 is a schematic diagram of an example base monitoring system with a wearable diaphragm sensor according to various example embodiments.

FIG. 2 is a perspective view of an example non-invasive wearable sensor showing a cable, buckle, and housing according to various example embodiments.

FIG. 3 is a chart showing an example of a tEAdi signal measured from a diaphragmatic sensor according to various example embodiments.

FIG. 4 is a surface plot showing an example three-dimensional tEAdi signal measured from a diaphragmatic sensor according to various example embodiments.

FIG. 5 is a block diagram of an AI-powered signal processing pipeline for deriving respiratory parameters from sensor data according to various example embodiments.

FIG. 6 is a block diagram of a system architecture integrating sensor outputs, machine learning algorithms, clinical records, and decision logic according to various example embodiments.

FIG. 7 is a flowchart of a machine learning model training process for respiratory classification according to various example embodiments.

FIG. 8 is a neural network diagram showing an example four-layer deep learning model for classifying respiratory conditions according to various example embodiments.

FIG. 9 is a chart showing an example of AI predictive model accuracy over successive learning iterations according to various example embodiments.

FIG. 10 is a diagram of an extended wearable belt incorporating multiple sensors for capturing respiratory and cardiac parameters according to various example embodiments.

FIG. 11 is a block diagram of a comprehensive patient monitoring system integrating respiratory, vital sign, and cardiac data according to various example embodiments.

FIG. 12 is a screenshot of a GUI showing real-time respiratory parameter bar graphs according to various example embodiments.

FIG. 13 is a chart showing an example respiratory rate waveform according to various example embodiments.

FIG. 14 is a chart showing an example tidal volume waveform according to various example embodiments.

FIG. 15 is a chart showing an example minute volume waveform according to various example embodiments.

FIG. 16 is a chart showing an example inspiratory-expiratory ratio (I:E ratio) waveform according to various example embodiments.

FIG. 17 is a chart showing an example rapid shallow breathing index (RSBI) waveform according to various example embodiments.

FIG. 18 is a layout diagram of a haptic feedback module embedded in a wearable sensor according to various example embodiments.

FIG. 19 is a block diagram of a cloud-based infrastructure for remote data routing and electronic health record integration according to various example embodiments.

FIG. 20 is a flowchart illustrating a therapy override protocol involving clinician confirmation according to various example embodiments.

FIG. 21 is a diagram of sensor fusion logic integrating CO₂ and temperature data into a respiratory model according to various example embodiments.

FIG. 22 is an exploded view of a wearable sensor showing a diaphragm sensor, accelerometer, housing, and patient interface components according to various example embodiments.

FIG. 23 is a logic diagram for detecting respiratory effort and oxygen saturation mismatches during dialysis according to various example embodiments.

FIG. 24 is a block diagram showing system integration for closed-loop ventilator control based on respiratory monitoring according to various example embodiments.

FIG. 25 is a block diagram illustrating system logic paths for therapy-assist mode and monitoring-only mode according to various example embodiments. The diagram includes inputs and outputs associated with each mode, as well as the conditions or triggers used to switch between operational modes.

FIG. 26 is a flowchart showing an example control loop for automated ventilator adjustment based on calculated rapid shallow breathing index (RSBI) according to various example embodiments. The flowchart includes signal input, RSBI computation, threshold evaluation, and ventilator parameter adjustment steps.

FIG. 27 is a diagram of a machine learning engine configured to perform patient-specific threshold adaptation based on historical respiratory data and population-level models according to various example embodiments. The diagram includes inputs for respiratory signals, baseline models, and clinical outcomes, and shows dynamic threshold recalibration logic.

FIG. 28 is a flowchart illustrating a therapy escalation decision protocol using multi-parameter trend analysis and severity scoring according to various example embodiments. The flowchart shows how the system may generate recommendations for therapy changes based on patient condition classification and parameter deviation thresholds.

### DETAILED DESCRIPTION

The following description provides specific details to illustrate example embodiments. It will be understood by those skilled in the art that the invention can be practiced with or without these details. Schematics, use cases, and diagrams are provided for clarity and are not limiting, as the invention is defined solely by the claims. Modifications and alternatives are possible within the scope of the invention, and features may be used independently or in combination.

Various example elements are referred to herein consistently with the following element numbers:

### FIG. 1—Base Monitoring System Schematic

100—Base Unit
102—Processor/GUI
104—360-Degree Alarm Visible from All Directions
106—Capacitive Touch Button
107—Wireless Transceiver
108—Lockable Connector (Base Unit Side)
109—System Cart Assembly
110—Cart Handle
112—System Cart
114—Power Supply Bracket
116—Cord Management Bracket
118—Cart Base
120—Lockable Wheels/Casters
122—Patient-Sensor cable
124—Lockable Connector (Wearable Sensor Side)

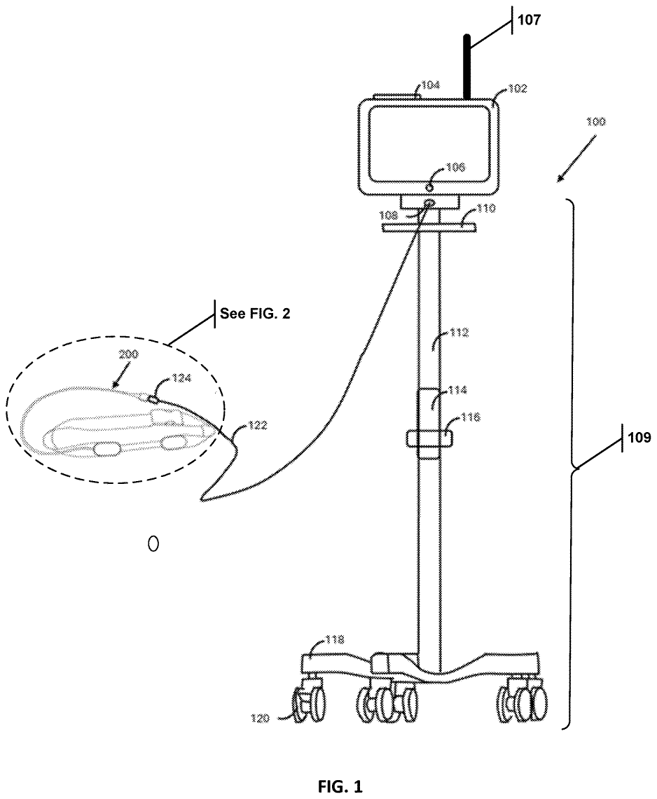

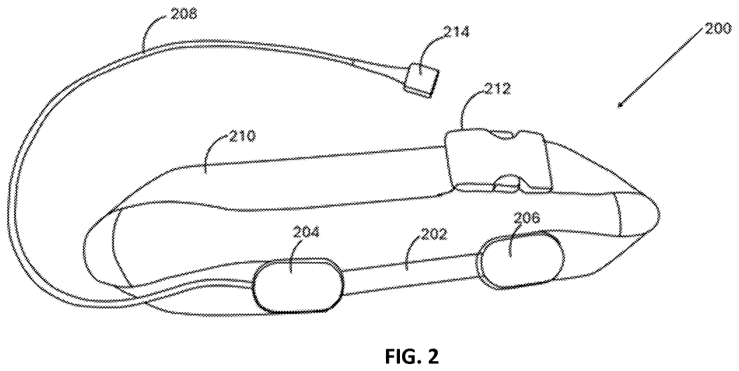

### FIG. 2—Wearable Sensor Perspective View

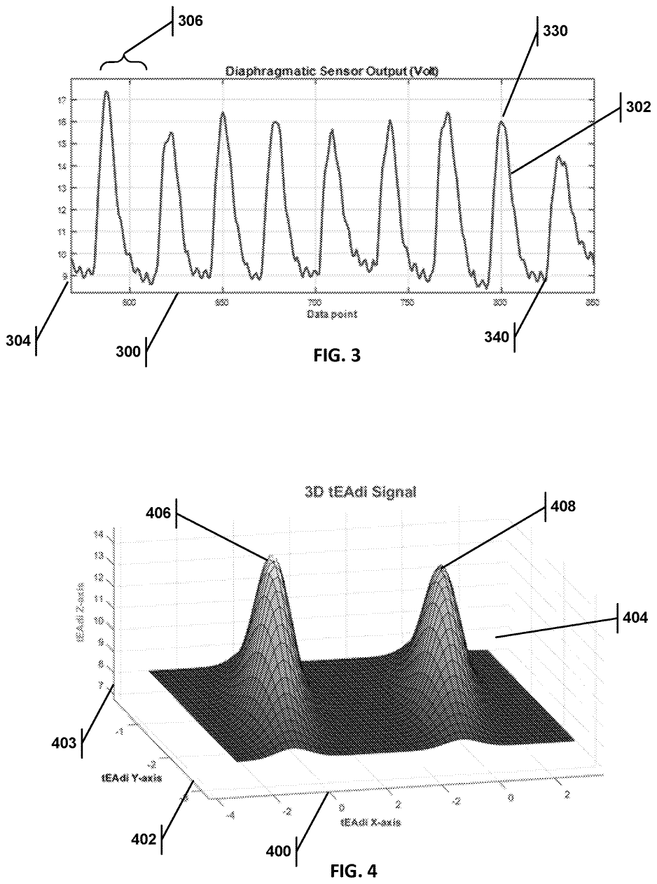

200—Wearable Diaphragm Sensor
202—Diaphragm Sensor
204—Sensor Housing
206—Processor
208—Cable
210—Adjustable Belt
212—Buckle
214—Lockable Connector
   FIG. 3—tEAdi Signal Chart
300—Time Axis
302—Waveform
304—tEAdi Signal Amplitude
306—Signal Patterns
330—Signal Peak Region
340—Noise Floor Reference
   FIGS. 4—3D tEAdi Surface Plot
400—Time Axis
402—Frequency Axis
403—Signal Amplitude
404—Signal Surface
408—Diaphragm Activation Hot Zone

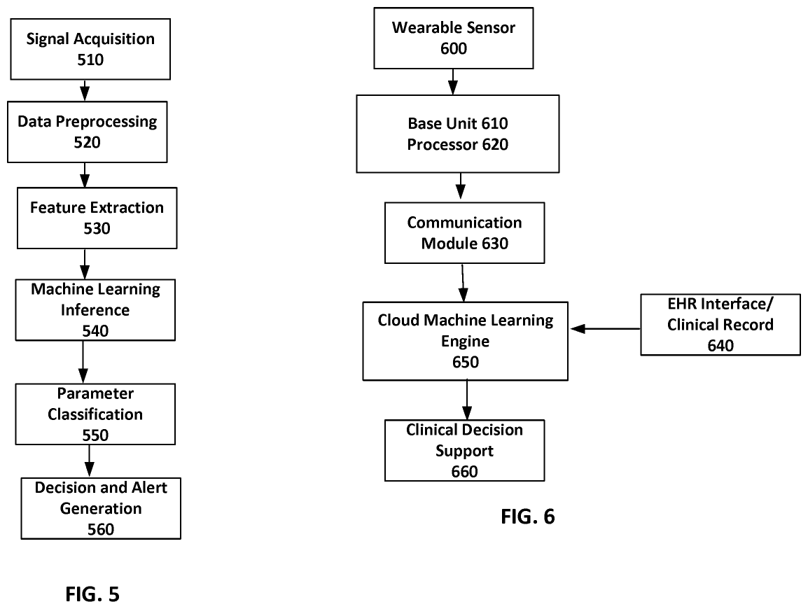

### FIG. 5—Signal Processing Pipeline Block Diagram

510—Signal Input/Acquisition (MMG/tEAdi)
520—Data Preprocessing
530—Feature Extraction
540—Machine Learning Inference
550—Parameter Classification
560—Alert Generation

### FIG. 6—Integrated System Architecture

600—Wearable Sensor
610—Base Unit
620—Local Processing Engine
630—Secure Communication Module
640—EHR Interface
650—Cloud ML Engine
660—Clinical Decision Support

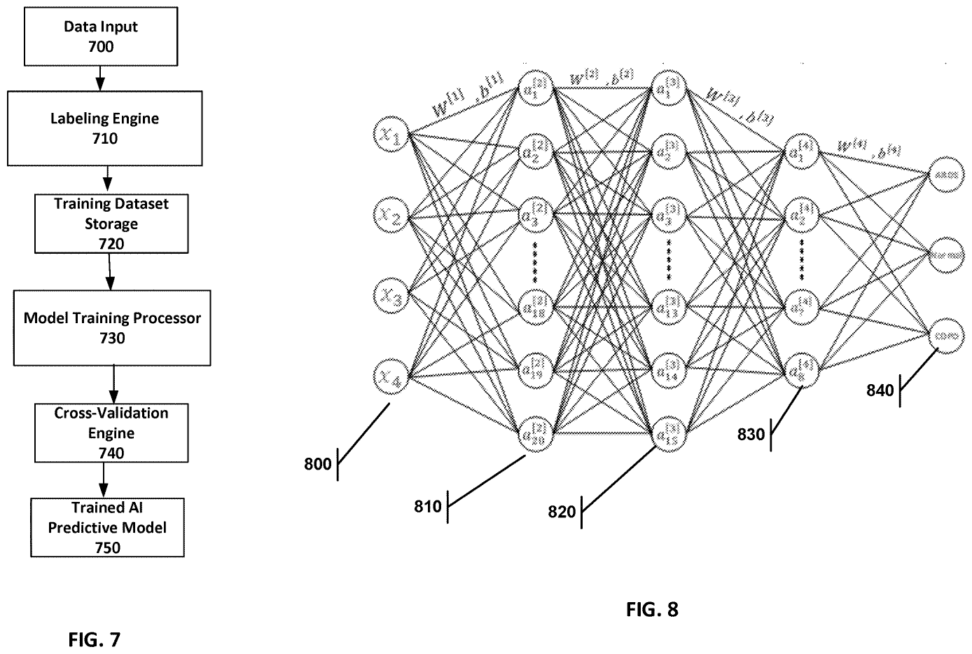

### FIG. 7—ML Model Training Flowchart

700—Input Data Stream
710—Labeling Engine
720—Training Dataset Storage
730—Model Training Processor
740—Cross-Validation Engine
750—Trained Model Export

### FIG. 8—Neural Network Architecture Diagram

800—Input Layer
810—First Hidden Layer
820—Second Hidden Layer
830—Third Hidden Layer
840—Output Layer

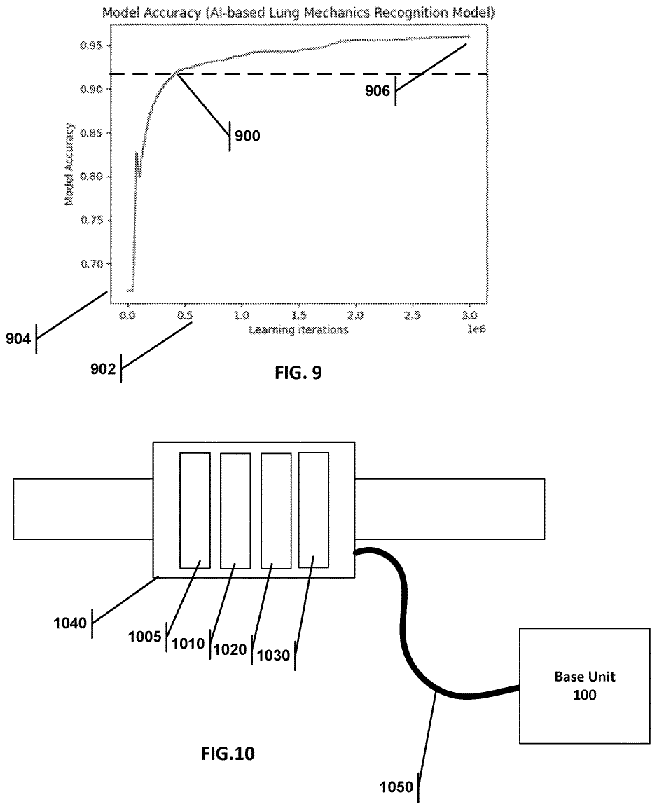

### FIG. 9—Model Accuracy Chart

900—Training Accuracy Curve
902—Iteration Axis
904—Accuracy Percentage Axis
906—Convergence

### FIG. 10—Extended Wearable Belt Design

1000—Belt Strap
1005—PPG Sensor
1010—MMG Sensor
1020—ECG Sensor
1030—SpO₂ Sensor
1040—Housing
1050—Multi-Sensor Cable Bundle

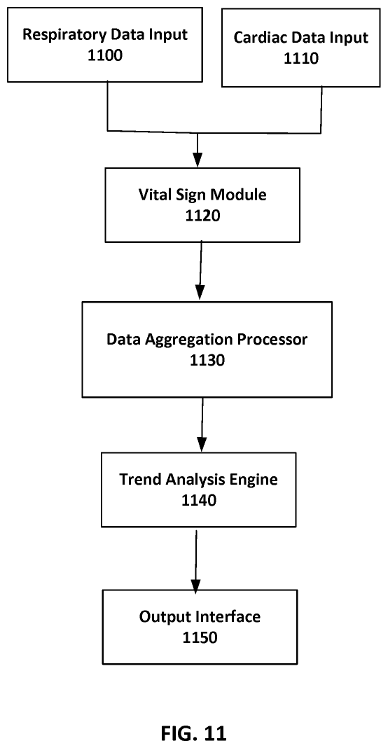

### FIG. 11—Comprehensive Monitoring Block Diagram

1100—Respiratory Input
1110—Cardiac Input
1120—Vital Sign Module
1130—Data Aggregation Processor
1140—Trend Analysis Engine
1150—Output Interface

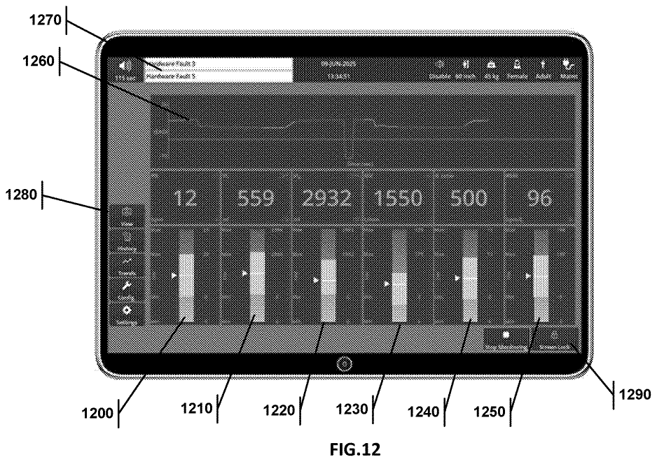

### FIG. 12—GUI Screenshot: Real-Time Bar Graphs

1200—Respiratory Rate Bar
1210—Tidal Volume Bar
1220—Minute Volume Bar
1230—RSBI Bar
1240—Color-Coding Legend
1250—Patient ID Display
1260—tEAdi Waveform
1270—Alert Indicator Panel
1280—Navigation Menu
1290—Screen Lock

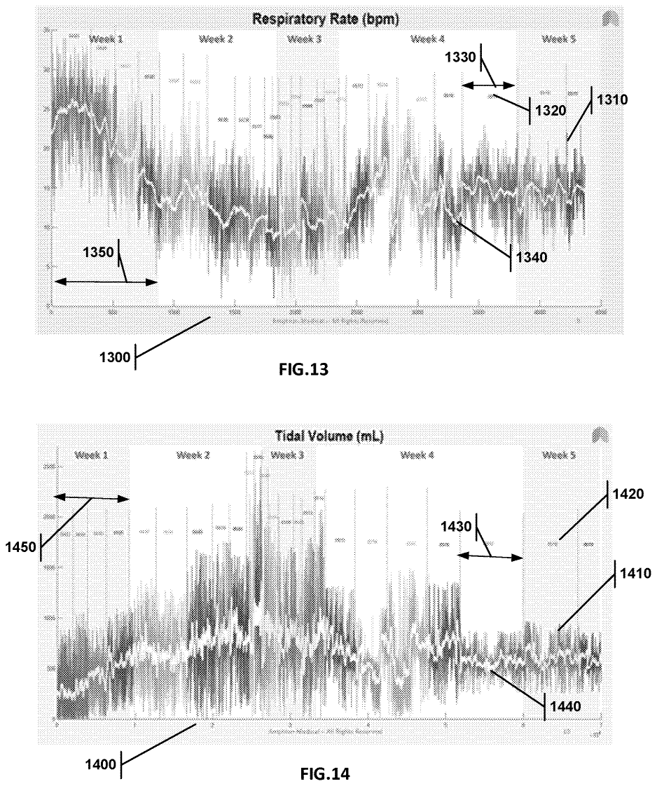

### FIG. 13—Respiratory Rate Waveform Chart

1300—Time Axis
1310—Respiratory Rate Signal
1320—Date Marker
1330—Event Segment
1340—Trend Smoothing Curve
1350—Week Band

### FIG. 14—Tidal Volume Waveform Chart

1400—Time Axis
1410—Tidal Volume Signal
1420—Date Marker
1430—Event Segment
1440—Trend Smoothing Curve
1450—Week Band

### FIG. 15—Minute Volume Waveform Chart

1500—Time Axis
1510—Minute Volume Signal
1520—Date Marker
1530—Event Segment
1540—Trend Smoothing Curve
1550—Week Band

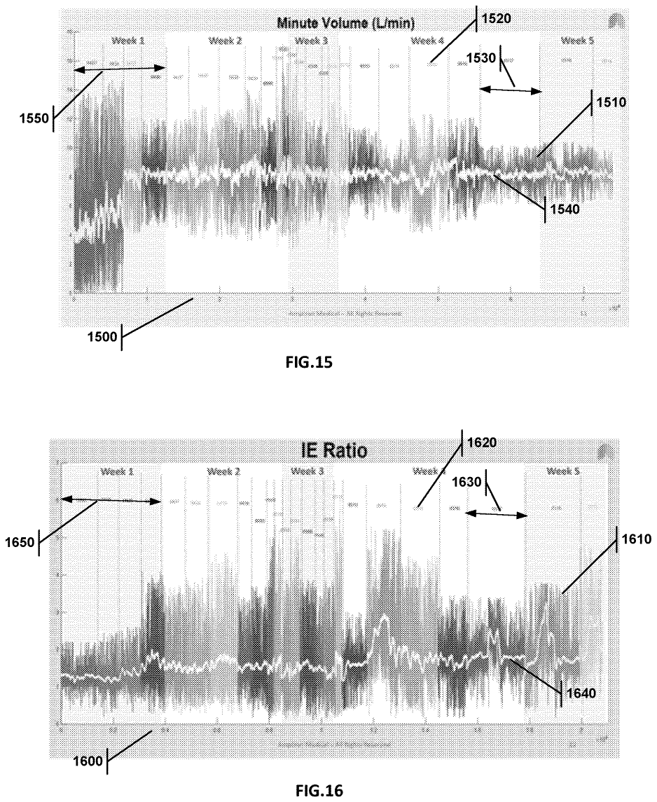

### FIG. 16—I:E Ratio Waveform Chart

1600—Time Axis
1610—I:E Ratio Signal
1620—Date Marker
1630—Event Segment
1640—Trend Smoothing Curve

### 1650—Week Band

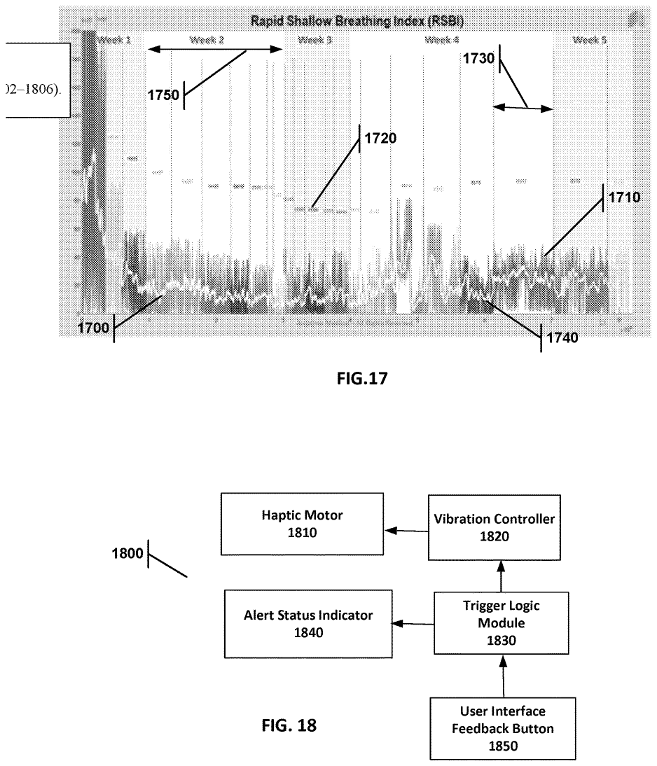

### FIG. 17—RSBI Waveform Chart

1700—Time Axis
1710—RSBI Signal
1720—Date Marker
1730—Event Segment
1740—Trend Smoothing Curve
1750—Week Band

### FIG. 18—Haptic Feedback Layout Diagram

1800—Wearable Housing
1810—Haptic Motor
1820—Vibration Controller
1830—Trigger Logic Module
1840—Alert Status LED
1850—User Interface Feedback Button

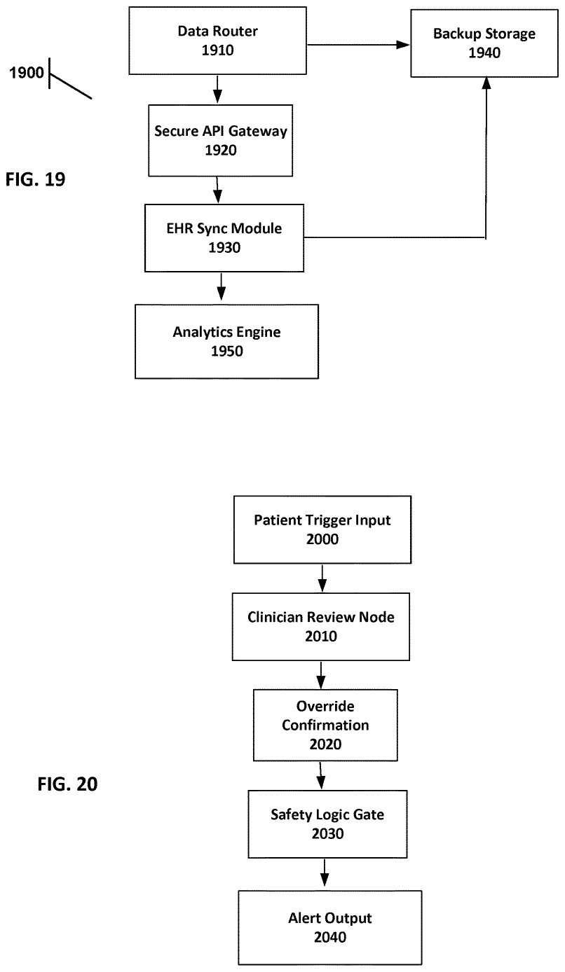

### FIG. 19—Cloud-Based Infrastructure Diagram

1900—Remote Monitoring Server
1910—Data Router
1920—Secure API Gateway
1930—EHR Sync Module
1940—Backup Storage Node
1950—Analytics Engine

### FIG. 20—Therapy Override Protocol Flowchart

2000—Patient Trigger Input
2010—Clinician Review Node
2020—Override Confirmation
2030—Safety Logic Gate
2040—Alert Output

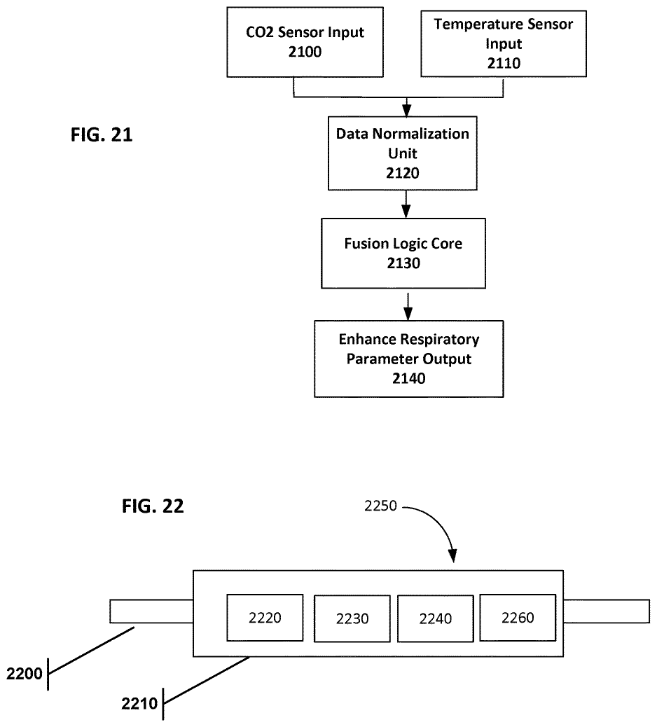

### FIG. 21—Sensor Fusion Logic Diagram

2100—CO₂ Sensor Input
2110—Temperature Sensor Input
2120—Data Normalization Unit
2130—Fusion Logic Core
2140—Enhanced Respiratory Parameter Output

### FIG. 22—Exploded Sensor View

2200—Belt Strap
2210—Outer Housing
2220—Diaphragm Sensor
2230—Accelerometer
2240—Signal Conditioning Board
2250—Skin Contact Pad
2260—Additional Components/Sensors

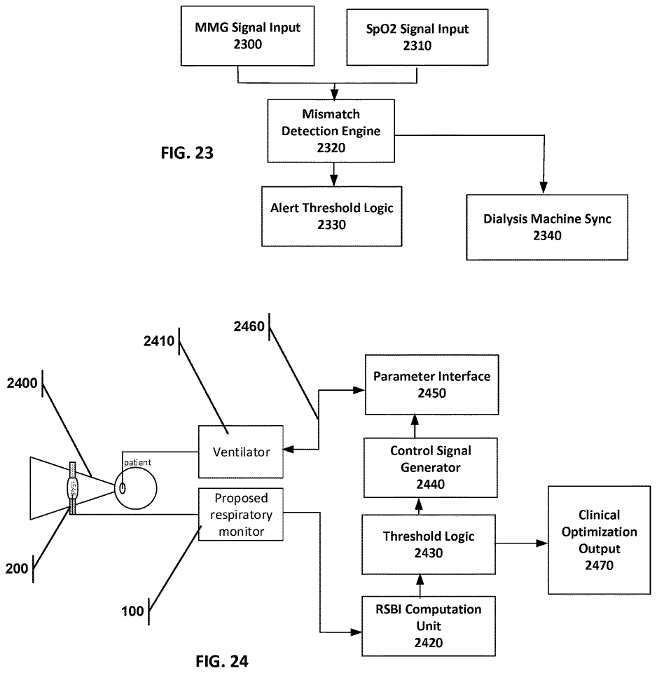

### FIG. 23—Dialysis Mismatch Logic Diagram

2300—MMG Signal Input
2310—SpO₂ Input
2320—Mismatch Detection Engine
2330—Alert Threshold Logic
2340—Dialysis Machine Sync

### FIG. 24—Closed-Loop Ventilator Control Diagram

2400—Patient
2420—RSBI Computation Unit
2430—Decision Threshold Logic
2440—Control Signal Generator
2450—Ventilator Parameter Interface
2460—Bidirectional Communication Link

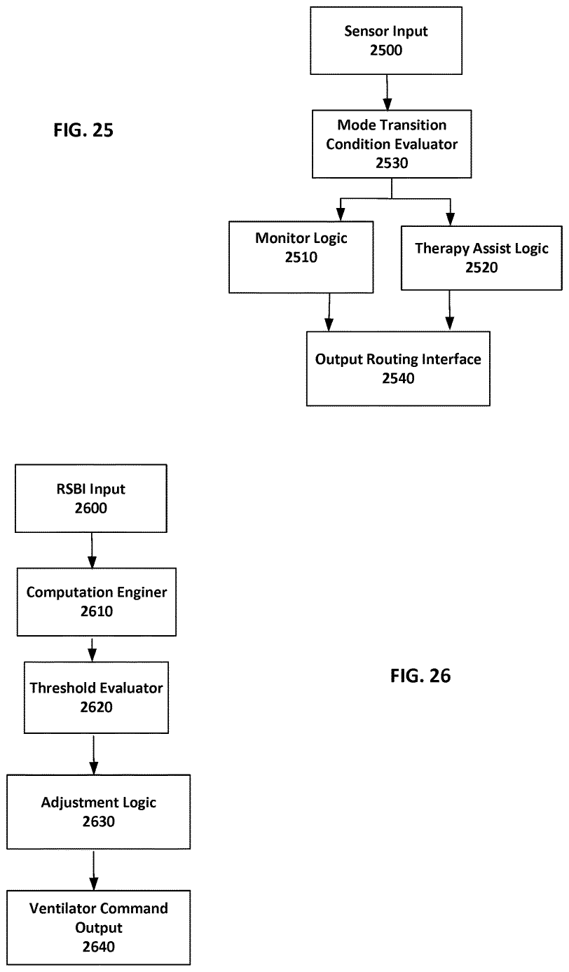

### FIG. 25—Therapy-Assist Logic Block Diagram

2500—Input Signal Set
2510—Therapy Mode Logic
2520—Monitoring Mode Logic
2530—Mode Transition Evaluator
2540—Output Routing Interface

### FIG. 26—RSBI-Based Control Loop Flowchart

2600—RSBI Input Node
2610—Computation Engine
2620—Threshold Evaluation
2630—Adjustment Logic
2640—Ventilator Command Output

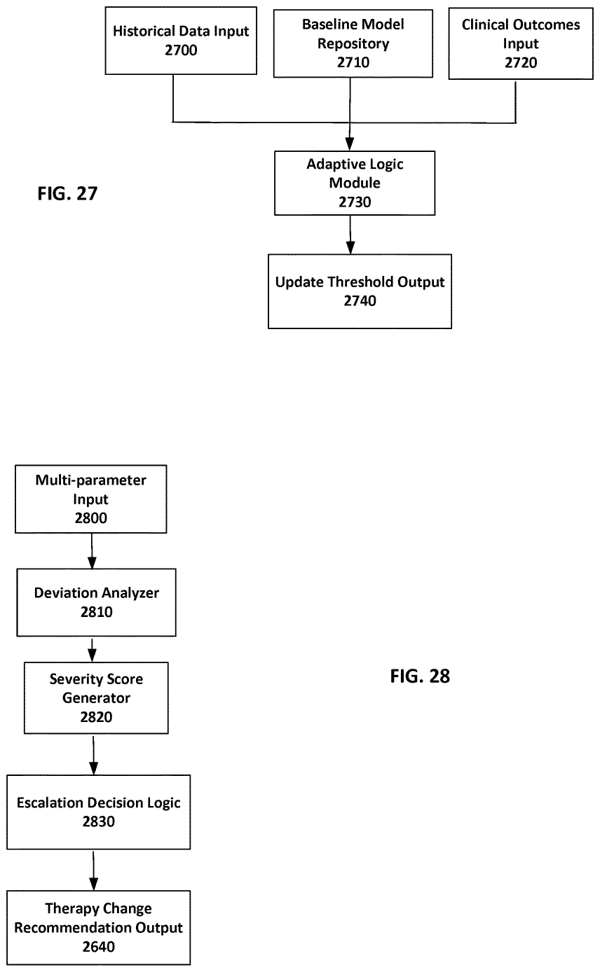

### FIG. 27—Patient-Specific Threshold Adaptation Diagram

2700—Historical Respiratory Data
2710—Baseline Model Repository
2720—Clinical Outcomes Input
2730—Adaptive Logic Module
2740—Updated Threshold Output

### FIG. 28—Therapy Escalation Protocol Flowchart

2800—Multi-Parameter Input Node
2810—Deviation Analyzer
2820—Severity Score Generator
2830—Escalation Decision Logic
2840—Therapy Change Recommendation

This description outlines example system components and how they work together to provide continuous respiratory monitoring in various example embodiments. Other example embodiments may include fewer, additional, or different components and features as would be apparent to persons of skill in the art. With reference to the figures and the identified example elements, example embodiments are now described in detail.

FIG. 1 illustrates a schematic diagram of an example base unit 100, which may serve as the central hub for processing, visualization, storage, therapy coordination, and communication of respiratory data. The base unit 100 may receive and analyze physiological signals transmitted from a wearable diaphragm sensor 200, which may be configured to monitor diaphragmatic motion and related parameters in real time.

The base unit 100 may include a processor with a GUI 102 configured to process incoming analog or digital signals. These signals may include, for example, mechanomyographic (MMG) signals, photoplethysmographic (PPG) and accelerometer-derived motion signals originating from an accelerometer within the wearable diaphragm sensor 200, or composite signals such as transcutaneous electrical activity of the diaphragm (tEAdi) signal. These input streams may be routed to a machine learning module, which may apply trained models to extract clinically relevant respiratory parameters from the data.

Signal input from the wearable diaphragm sensor 200 may be received via a cable 122 to base unit 100, which physically connects to the lockable sensor connector 108 on the base unit 100, and 124 on the wearable sensor. In some embodiments, the lockable sensor connectors 108, 124 may comprise a keyed, magnetic, or twist-lock mechanism to prevent unintentional disconnection and to support safe patient mobility or rapid detachment in emergencies. This two-piece cable connector design allows the patient to disconnect from the system without removing the wearable sensor.

The GUI 102 may be provided on the base unit 100 to enable clinician interaction with the system. The GUI 102 may present real-time respiratory waveforms (e.g., as in FIG. 3), time-aligned trend graphs, therapy overlays, alert indicators, and patient metadata (e.g., as in FIG. 12). In various embodiments, the GUI 102 may support capacitive touch, membrane buttons, or external control inputs to allow clinicians to annotate signals, review respiratory events, configure therapy options, or document symptoms.

An alert logic module, which may be a part of the processor 102, may generate visual, auditory, or haptic alerts when one or more derived respiratory parameters exceed predefined thresholds. In some embodiments, the alert logic module may drive multi-angle visual indicators or output alert metadata to connected dashboards or mobile interfaces (see FIGS. 12, 18 and 20). Alerts may be ranked by severity, color-coded by type, and synchronized across connected devices. A 360-degree visible alarm 104 may be visible from all direction.

A capacitive touch button 106 may be positioned on the housing of the base unit 100 to provide the user with direct physical control over common operations, such as acknowledging alerts, pausing live monitoring, toggling display views, or triggering a wireless data sync. The capacitive touch button 106 may be designed with soft-touch or sealed membrane construction to maintain cleanability and support infection control protocols.

The wireless transceiver 107 may enable encrypted communication between the base unit 100 and remote endpoints, including cloud dashboards, mobile caregiver applications, and clinical electronic health records. Data streams or inference outputs may be routed to a cloud interface module, which is part of the processor 102 and may support long-term data storage, clinician portal access, population-level reporting, or AI model retraining workflows (see FIGS. 6, 7, and 27).

A therapy control module, also part of the processor 102, may coordinate operation with one or more therapy devices, including ventilators, airway pressure systems, or neuromuscular stimulators. In embodiments where closed-loop control is used, the therapy control module may communicate with a ventilator interface module to modulate device parameters in real time based on respiratory effort metrics, patient-specific thresholds, or rapid shallow breathing index (RSBI) scores (see FIGS. 24 and 28).

A symptom tracking module, also a part of the processor 102, may also be included within the base unit 100, configured to store, tag, and visualize user-entered symptoms, questionnaire results, or clinician observations. These data may be manually entered via the GUI 102 or synchronized from a mobile interface, and may be used in conjunction with sensed data to support context-aware analysis, therapy personalization, or clinical documentation (see FIGS. 27 and 28).

The base unit 100 may be physically mounted on a system cart assembly 109, which may provide mobility, access, and ergonomic positioning in both bedside and ambulatory use cases. The system cart assembly 109 may include a cart handle 110 for repositioning, a cart base 118 for structural stability, and lockable wheels 120 to prevent unintended movement during patient care.

Additional support features may include a power supply bracket 114, which may be configured to hold an external AC adapter or battery pack, and a cord management bracket 116, which may allow coiled storage of the cable 122 to base unit 100 when not in use. These structural components may reduce cable clutter, minimize trip hazards, and support a clean clinical workflow.

In summary, the base unit 100, as illustrated in FIG. 1, may function as a portable, modular hub for acquiring, analyzing, displaying, and routing respiratory signals and related data. In various configurations, it may operate independently or as part of an integrated monitoring and therapy platform involving cloud systems, mobile devices, and third-party equipment. Embodiments may include fewer, additional, or alternative modules depending on the clinical use case.

FIG. 2 illustrates a perspective view of an example wearable diaphragm sensor 200 that may be configured to acquire physiological data from a region proximate the patient's diaphragm, such as the upper abdomen (thoracoabdominal muscles). Other placements may be used depending on patient anatomy or clinical goals. The wearable diaphragm sensor 200 may be secured to the patient using a circumferential adjustable belt 210 and may be designed for continuous or intermittent use in clinical or home environments.

In the illustrated embodiment, the wearable diaphragm sensor 200 may include a sensor housing 204, which may be contoured to fit the patient's body. The sensor housing 204 may contain a diaphragm sensor 202, which may include one or more flexible capacitive or stretch sensors configured to deform with diaphragmatic motion. The diaphragm sensor 202 may include a flexible capacitive sensor capable of capturing high-resolution respiratory effort signals from the patient's abdominal surface. The diaphragm sensor 202 may be constructed from stretchable, biocompatible materials such as medical-grade silicone or polyurethane, and may incorporate dielectric electroactive polymers that change capacitance in response to mechanical dimensions. The diaphragm sensor 202 may detect multi-dimensional deformation in X, Y, and Z axes, enabling the more accurate inference of respiratory effort and breathing patterns.

The sensor housing 204 may also enclose a set of onboard electronics. These may include an accelerometer configured to detect body position, movement artifacts, or changes in patient orientation. Data from the accelerometer may be used to augment signal quality analysis, adapt ML thresholds, or detect postural influences on respiratory effort.

A processor 206 may include a signal conditioning circuitry that may amplify and filter analog signals from the diaphragm sensor element 202, and a microcontroller that may digitize and encode this data for transmission. A local power supply, such as a rechargeable battery, may support autonomous operation. In some configurations, a wireless communication module and an antenna may also be included in the sensor housing 204, allowing untethered data transmission to the base unit 100.

For wired operation, a cable 208 may be integrated into the sensor housing 204, and a lockable connector 214 may be provided to secure the wired connection. A cable 122 to base unit (see FIG. 1) may transmit data and power between the wearable diaphragm sensor 200 and the base unit 100.

The sensor may interface non-invasively with the patient's body through their clothing, which can improve ease of use and comfort. The assembly may be secured to the patient using the belt 210, which may include an adjustable buckle 212 or a fastening pad such as a Velcro® or hook-and-loop closure.

FIG. 3 illustrates an example tEAdi signal waveform 302, which may represent a non-invasively measured signal indicative of transcutaneous electrical or mechanical activity of the diaphragm. The tEAdi signal waveform 302 may be acquired using a diaphragm sensor 202, positioned within a wearable diaphragm sensor 200 that is located over the upper abdomen or other region proximate to the diaphragm depending on patient anatomy, respiratory needs, or sensor configuration.

Together, the components of FIG. 2 form a compact, non-invasive sensing platform capable of generating high-resolution respiratory signals for use in real-time monitoring, clinical decision support, and therapy coordination. The integration of the diaphragm sensor element 202, the accelerometer, and the supporting electronics within the sensor housing 204 provides robust signal acquisition across diverse environments. In some embodiments, the wearable diaphragm sensor 200 may be embedded in garments, flexible straps, or patient harnesses depending on mobility needs, comfort preferences, or concealment requirements.

In various embodiments, the tEAdi signal waveform 302 may reflect mechanical and/or electrical activation of the diaphragm muscle, captured through one or more sensing modalities, including but not limited to: flexible capacitive sensors, piezoelectric elements, strain gauges, mechanomyographic sensors, or impedance-based electrodes. The system may combine these modalities to improve robustness against motion artifacts and postural variation.

The term “tEAdi” as used herein refers to a signal acquired transcutaneously and non-invasively, in contrast to invasive neural respiratory drive sensors. The tEAdi signal waveform 302 may be correlated with diaphragm contractility and effort, providing a direct measurement of patient breathing work and timing. In some embodiments, the tEAdi signal waveform 302 may include contributions from both mechanical deformation and electrical impedance variation, depending on how the diaphragm sensor 202 is constructed and implemented.

The tEAdi signal waveform 302 in FIG. 3 may be plotted against a time axis 300, extending horizontally, and an amplitude axis 304, extending vertically. The amplitude axis 304 may reflect signal strength or deflection corresponding to diaphragmatic activity, while the time axis 300 may represent seconds, milliseconds, or other sampling intervals depending on the system resolution and processing rate.

Within the tEAdi signal waveform 302, repeating segments known as signal patterns 306 may be visible. Each signal pattern 306 may correspond to one or more respiratory cycles and may contain identifiable features representing the inspiratory and expiratory phases of breathing. The shape, slope, frequency, and duration of the signal patterns 306 may be analyzed to determine respiratory effort, rhythm, rate, and consistency. The waveform 302 has a signal peak 330 and a noise floor reference 340.

In operation, the base unit 100 may analyze the tEAdi signal waveform 302 and extract any combination of derived respiratory parameters, including but not limited to: respiratory rate, inspiratory time, expiratory time, I:E ratio, inspired tidal volume (VTi), expired tidal volume (VTe), minute ventilation (MV), rapid shallow breathing index (RSBI), cough detection, effort-recovery time, and abnormal rhythm events. Such computations may be performed by the machine learning module within the processor/GUI 102 and/or transmitted to the cloud interface module for enhanced inference or longitudinal tracking.

Abnormalities in the signal patterns 306, such as low amplitude, irregular spacing, or waveform flattening, may indicate respiratory muscle fatigue, neuromuscular impairment, or clinical signs of dyspnea or apnea. These anomalies may trigger alerts via the alert logic module 102 or be flagged for clinician review through the GUI 102.

In some embodiments, the tEAdi signal waveform 302 may be displayed in real time on the GUI 102, optionally with overlays, annotations, and synchronized event markers (see FIGS. 4 and 12). When a critical threshold is crossed, the alert logic module may issue an audiovisual notification or transmit alert data through the wireless transmitter 106 to mobile caregivers or cloud-connected dashboards.

FIG. 3 thus illustrates a foundational diagnostic and analytical signal within the system. By enabling continuous, non-invasive, high-resolution monitoring of diaphragm activation, the tEAdi signal waveform 302 may support early detection of clinical deterioration, guide ventilator weaning, or track the effectiveness of therapy interventions in patients with COPD, neuromuscular disease, or acute respiratory failure.

FIG. 4 illustrates an example signal amplitude surface 404, representing a three-dimensional visualization of diaphragm activation derived from a diaphragm sensor 202, as described in FIG. 2. The signal amplitude surface 404 may represent spatial and temporal variations in mechanical or electrical activity of the diaphragm muscle during breathing cycles. This three-dimensional representation may provide enhanced insight into respiratory mechanics relative to conventional one-dimensional waveform plots.

The signal amplitude surface 404 may be computed from multidimensional inputs collected by the diaphragm sensor 202 and the accelerometer, both enclosed within the sensor housing 204. These data streams may reflect changes in capacitance, stretch, strain, acceleration, or impedance, and may be sampled across multiple axes corresponding to anatomical planes of diaphragmatic motion: mediolateral, superoinferior, and anteroposterior.

The horizontal dimensions of the surface may be defined by a time axis 400 and a frequency axis 402. In various embodiments, the time axis 400 may reflect temporal progression in seconds or sampling indices, while the frequency axis 402 may represent either spectral features derived from signal decomposition or spatial sensor orientation (e.g., directional vector alignment or electrode location). The signal amplitude surface 404 may be rendered across these two axes to reflect dynamic changes over time and space.

The vertical displacement of the signal amplitude surface 404 may encode signal amplitude, relative contraction force, or composite waveform magnitude, and may correspond to fused signal scores derived from MMG and accelerometric modalities. In some embodiments, a color map legend 406 may be applied to enhance visualization of magnitude or abnormality severity, using continuous or segmented scales. Specific regions of elevated activation may be referred to as a diaphragm activation hot zone 408, denoting peak inspiratory effort or signal features of clinical interest.

Together, the time axis 400, frequency axis 402, and signal amplitude surface 404 define a three-dimensional surface visualization of diaphragmatic activity, with distinct patterns indicating the presence, shape, and symmetry of inspiratory-expiratory cycles. In healthy subjects, the signal amplitude surface 404 may demonstrate regular, symmetrical undulations. Deviations in topology may indicate one or more of the following:
Diaphragm fatigue, atrophy, or disuse
Paradoxical or asynchronous breathing
Positional respiratory impairment
Early signs of ventilator-patient mismatch
Exacerbation of obstructive pulmonary conditions (e.g., COPD)

The base unit 100 may render the signal amplitude surface 404 in real time via the GUI 102, allowing clinicians to view diaphragm activation dynamically. In other embodiments, the signal surface may be processed by the machine learning module within the processor/GUI 102 or uploaded to the cloud interface module for extraction of spatial signal features, trend analysis, or classification using trained neural networks.

The diaphragm activation hot zone 408 may be monitored for displacement, expansion, or flattening, and such changes may correlate with altered respiratory mechanics or reduced inspiratory strength. When threshold deviations are detected, the alert logic module 102 may initiate a local or remote alert based on configured severity levels.

The signal amplitude surface 404 shown in FIG. 4 may serve as a complement to the tEAdi signal waveform 302 shown in FIG. 3. While the tEAdi signal waveform 302 provides high-resolution temporal insights, the signal amplitude surface 404 enhances interpretability by presenting multi-axis spatial variation. This combination may allow for earlier recognition of deteriorating patient status, more robust ventilator titration, or improved diagnostic sensitivity in conditions such as neuromuscular weakness, paradoxical breathing, and patient-ventilator dyssynchrony.

FIG. 5 illustrates a block diagram of an example signal processing pipeline, comprising a multi-stage algorithmic workflow executed by the system to derive respiratory parameters from physiological sensor data. This pipeline may be implemented in whole or in part within the processor 102 of the base unit 100, or may be deployed across both local and remote infrastructure using modular software containers, firmware modules, or cloud-deployed services.

The pipeline may begin at the signal acquisition block 510, which may be configured to receive input from the wearable diaphragm sensor 200, including the diaphragm sensor 202, the accelerometer, and, in some configurations, additional physiological sensors (e.g., temperature, PPG, impedance). The acquired data may include a tEAdi signal waveform 302, accelerometric traces, or composite signals that reflect both electrical and mechanical activity of the diaphragm and upper thoracic region.

The incoming raw data may be passed to a preprocessing block 520, which may normalize, align, filter, and resample signals from different sources. The preprocessing block 520 may also perform baseline correction, motion artifact rejection, denoising, channel synchronization, and other signal hygiene techniques. In some embodiments, signal quality may be dynamically assessed based on posture, recent motion events, or sensor contact or placement quality inferred from the accelerometer or diaphragm sensor 202.

From there, signals may be routed to a feature extraction block 530, where waveform characteristics such as peak amplitude, rise time, cycle duration, inter-breath intervals, and frequency content may be calculated. The feature extraction block 530 may compute derived metrics over moving windows, breath-by-breath intervals, or cumulative time segments, optionally using thresholds or adaptive baselines for personalization.

The extracted features may be fed into a machine learning inference block 540, which may apply a trained model to the incoming data. The model may take the form of a deep neural network, convolutional classifier, random forest, or ensemble learning architecture, and may be trained using representative data from healthy individuals and those with known respiratory conditions such as COPD, asthma, neuromuscular disease, or COVID-related lung injury. In some configurations, the machine learning inference block 540 may be capable of online learning or continuous refinement using anonymized, cloud-stored patient data accessed via the cloud interface module.

The output of the machine learning inference block 540 may be processed by a parameter classification block 550, which may assign quantitative values or labels to respiratory events or time segments. These parameters may include, for example:
Respiratory rate (RR)
Inspiratory and expiratory time
Inspiratory-to-expiratory (I:E) ratio
Inspired tidal volume (VTi)
Expired tidal volume (VTe)
Minute volume (MV)
Respiratory mechanics (RM) indicators
Rapid shallow breathing index (RSBI)
Presence of cough or double breath signatures
Indicators of dyspnea or effort intolerance

These values may be provided in real time to the GUI 102 for clinician visualization, logged for trend analysis, or passed downstream to therapeutic systems or decision engines.

The final stage of the pipeline may be implemented in a decision logic and alert generation block 560, which may compare respiratory parameter outputs to preconfigured thresholds, historical trends, or patient-specific baselines. When clinically relevant deviations are detected, the decision logic and alert generation block 560 may trigger alerts via the alert logic module, annotate the display of the GUI 102, or transmit structured alerts to mobile caregivers or cloud monitoring dashboards.

In some embodiments, alerting behavior may be modulated based on patient posture, recent activity patterns, or user-reported symptoms collected via the symptom tracking module. Alerts may also trigger re-evaluation of therapy setpoints by the therapy control module or other components interfaced via the ventilator interface module 2450.

The AI-enabled signal processing pipeline shown in FIG. 5 enables the system to function as a dynamic, context-aware, and adaptive respiratory analysis engine. Through this pipeline, the system may support continuous monitoring, real-time diagnosis, automated alerting, and personalized therapy management in a wide range of clinical and non-clinical environments.

FIG. 6 illustrates an example system-level architecture comprising multiple interconnected processing modules for integrating physiological sensor data, machine learning analytics, and clinical decision-making. This integrated system architecture may enable real-time or near-real-time respiratory assessment and therapy guidance across local and distributed environments. The configuration shown in FIG. 6 may be implemented using edge processing, cloud computing, or a hybrid model depending on deployment requirements.

The system may begin with wearable sensor input 600, which may originate from the wearable diaphragm sensor 200 described in FIG. 2. The wearable sensor input 600 may include signals acquired from the diaphragm sensor 202 and accelerometer, such as transcutaneous electrical activity of the diaphragm (tEAdi), mechanomyographic (MMG) signals, or accelerometric motion traces. These signals may reflect mechanical strain, vibrations, muscle activation, or postural shifts and may be transmitted to downstream processing modules via wired or wireless connection.

Signal data from the wearable sensor input 600 may be received by the base unit 610, which may correspond to the base unit 100 shown in FIG. 1. The base unit 610 may perform local filtering, buffering, encryption, and transmission tasks. The base unit 610 may also provide real-time display capabilities through the GUI 102, enabling clinicians to monitor raw signals, waveform overlays, and trend indicators.

Within the base unit 610, data may be routed to a local processing engine 620, which may execute onboard software modules such as the preprocessing block 520 and feature extraction block 530 described in FIG. 5. These operations may include signal normalization, denoising, and segmentation to prepare data for inference.

Processed signals may be transmitted securely via a secure communication module 630, which may handle bidirectional, encrypted data exchange with cloud-based services. The secure communication module 630 may also manage remote configuration updates, synchronization with mobile dashboards, and network health reporting.

Data and metadata from the base unit 610 may be transmitted to a cloud machine learning engine 650, which may implement the functionality described with reference to the machine learning inference block 540 of FIG. 5. The cloud machine learning engine 650 may include one or more trained models configured to classify respiratory patterns, predict clinical deterioration, or recommend therapy actions based on both real-time inputs and historical records.

The cloud machine learning engine 650 may also incorporate a feedback loop using historical datasets, including a structured electronic health record (EHR) interface 640. The EHR interface 640 may provide access to longitudinal clinical information such as prior diagnoses, therapy plans, outcomes, and clinician annotations. In some configurations, the cloud machine learning engine 650 may use federated learning, ensemble retraining, or dynamic thresholding strategies based on this EHR context.

Predictions, classifications, or parameter outputs may be transmitted from the cloud machine learning engine 650 to a clinical decision support module 660. The clinical decision support module 660 may serve as an integration layer, combining model outputs with configured clinical rules, patient-specific preferences, and guideline-driven constraints. The module may prioritize recommendations by severity, rank outcomes by risk class, and support explainability by surfacing features or signal segments most relevant to the model's prediction.

Recommendations generated by the clinical decision support module 660 may be presented through the GUI 102, allowing a clinician to review results, accept or override decisions, and optionally document their actions. These clinician-entered decisions may be routed back into the EHR interface 640, closing the loop and enabling ongoing model validation and clinical learning.

The system architecture depicted in FIG. 6 supports deployment in high-acuity inpatient settings, outpatient clinics, or remote monitoring workflows. It may enable early detection of conditions such as respiratory fatigue or COPD exacerbation through longitudinal trend analysis and patient-specific modeling. The system may also support integration with mobile caregiver platforms and telemedicine dashboards.

It will be appreciated that FIG. 6 provides only one representative arrangement of system modules. Alternative configurations may include additional components such as signal fusion engines, regulatory logging interfaces, user access control modules, or device orchestration tools. Individual modules may be instantiated in hardware, firmware, or cloud microservices, and may operate asynchronously, continuously, or on a scheduled batch basis.

FIG. 7 illustrates a flowchart representing an example architecture for training a machine learning model used to classify respiratory conditions, compute parameter estimates, or predict risk scores. The diagram depicts a modular process for developing a predictive model from historical or synthetic respiratory data. The model training architecture may be deployed locally on a clinical server, in a remote cloud environment, or distributed across a federated healthcare system using secure data aggregation.

The process may begin with an input data stream 700, which may include labeled physiological data collected using the wearable diaphragm sensor 200, including signals from the diaphragm sensor 202 and the accelerometer (see FIG. 2). The input data stream 700 may comprise time-series recordings representing a range of respiratory phenotypes, including healthy breathing patterns, obstructive lung disease, restrictive ventilatory disorders, and neuromuscular impairment.

In some configurations, the input data stream 700 may include contextual signals such as posture, body temperature, carbon dioxide concentration, oxygen saturation, or heart rate variability. The data may be recorded from patients in clinical trials, home settings, rehabilitation programs, or intensive care environments. Synthetic or augmented data may also be generated through signal warping, noise injection, or simulated pathology insertion to expand the diversity and robustness of the training corpus.

The input data stream 700 may be processed by a labeling engine 710, which may assign ground-truth classifications, symptom tags, or parameter ranges to each segment of the input data. Labeling may be performed manually by clinicians, semi-automatically via heuristics, or automatically using consensus models. In some embodiments, the labeling engine 710 may include tools for visual annotation, respiratory phase segmentation, or template matching.

Labeled data may be stored in a training dataset storage 720, which may serve as a structured repository for batch access by downstream training pipelines. The training dataset storage 720 may include indexed data formats, metadata files, and cohort labels, and may optionally support versioning, snapshotting, or access control for research and clinical compliance.

The data may be processed by a model training processor 730, which may execute a training algorithm such as a neural network, convolutional classifier, transformer-based model, or recurrent sequence model. The model training processor 730 may use supervised, unsupervised, or semi-supervised learning depending on availability of ground-truth labels. Transfer learning or federated learning strategies may be applied to refine general-purpose base models for specific clinical populations or conditions.

As part of training, the data may be passed through a cross-validation engine 740, which may perform k-fold cross-validation, holdout validation, or stratified sampling to evaluate model generalization and reduce overfitting. The cross-validation engine 740 may output validation metrics including accuracy, recall, precision, AUC, or FI score across diagnostic categories.

Upon successful training and validation, the resulting model may be passed to a trained model export 750, which may generate a portable, deployable format for integration into downstream inference pipelines (e.g., machine learning inference block 540 in FIG. 5 or cloud machine learning engine 650 in FIG. 6). The trained model export 750 may also store metadata such as model version, training parameters, validation history, and applicable population cohort.

The exported model may be used to produce predictive outputs, such as condition classifications, regression-based parameter estimates, or probabilistic risk scores. These outputs may be used in the clinical decision support module 660 (FIG. 6), routed to the GUI 102, or integrated with third-party health analytics platforms. In various embodiments, model outputs may include:
Classification of respiratory mechanics (e.g., obstructive, restrictive, normal)
Confidence scores or probability distributions
Time-to-deterioration predictions
Personalized parameter forecasts (e.g., expected RSBI threshold crossing)

This machine learning module may include an inference engine configured to classify diaphragm fatigue vs. central hypoventilation based on MMG-SpO₂ divergence and/or sensitivity comparison.

While FIG. 7 depicts a simplified flow from input data stream 700 to trained model export 750, other configurations may include additional preprocessing layers, signal augmentation modules, hyperparameter tuning, ensemble generation, or human-in-the-loop review. In some embodiments, training may be performed periodically using rolling datasets from newly acquired patient data, enabling continuous model improvement while maintaining explainability and auditability.

FIG. 8 illustrates an example architecture of a deep learning model featuring an input layer 800, one or more hidden layers, and an output layer 840. This architecture may be used for recognizing lung mechanics or classifying respiratory conditions. The diagram provides a non-limiting example of a fully connected feedforward neural network comprising multiple layers of interconnected nodes—commonly referred to as “neurons”—through which input data may be processed, transformed, and ultimately classified by a trained AI model.

The input layer 800 may receive a plurality of input features, represented in this example as x₁, x₂, x₃, x₄. These input features may be derived from any number of physiological signals or contextual data collected by one or more wearable diaphragm sensors 200 (see FIG. 2), including but not limited to outputs from the diaphragm sensor 202 and the accelerometer. The features may include raw or preprocessed signals reflecting respiratory motion, waveform amplitude, timing intervals, breathing effort, or multimodal parameters such as carbon dioxide (CO₂), oxygen saturation (SpO₂), body temperature, or patient posture. The total number and nature of input features may vary depending on system configuration and use case.

Between the input layer 800 and the first hidden layer 810, each input trainable feature may be multiplied by a weight matrix W^{{[1]}} and summed with a bias vector b^{{[1]}}, resulting in the first layer of transformed values. These parameters—weights and biases—constitute the learnable part of the network and are optimized during training to minimize classification error.

The first hidden layer 810 includes a plurality of artificial neurons, shown in this example as

$${a}_{1}^{\{ [ 2 ] \}} , {a}_{2}^{\{ [ 2 ] \}} , ... , {a}_{\{ 20 \}}^{\{ [ 2 ] \}} ,$$

corresponding to twenty nodes. Each neuron computes a weighted sum of its inputs and applies a nonlinear activation function, which may be a rectified linear unit (ReLU), sigmoid, hyperbolic tangent (tanh), or another activation function commonly used in neural networks. These non-linear operations enable the model to capture complex and subtle patterns within high-dimensional sensor data.

The outputs of the first hidden layer 810 are propagated to the second hidden layer 820, which includes fifteen neurons labeled

$${a}_{1}^{\{ [ 3 ] \}} , {a}_{2}^{\{ [ 3 ] \}} , ... , {a}_{\{ 15 \}}^{\{ [ 3 ] \}} .$$

The transformation at this stage again involves a set of weights W^{{[2]}} and biases b^{{[2]}}, and the application of an activation function to each resulting sum. This layer may represent intermediate feature abstractions or compression of low-level input patterns.

Subsequently, values are passed to the third hidden layer 830, which includes eight neurons

$${a}_{1}^{\{ [ 4 ] \}} , {a}_{2}^{\{ [ 4 ] \}} , ... , {a}_{8}^{\{ [ 4 ] \}} .$$

The third hidden layer 830 may compute higher-level features that are most strongly associated with a classification outcome. The progressive reduction in neuron count across layers—from 20 to 15 to 8—is illustrative and may be adjusted depending on task complexity, training data, or deployment constraints. In other embodiments, additional or fewer hidden layers, alternative architectures (e.g., convolutional, recurrent, or transformer blocks), or different connection patterns may be used.

The final layer in the model is the output layer 840, which receives the processed activations from the third hidden layer 830 and computes one or more output predictions ŷ. In the example shown, the output layer 840 classifies input data into several categories, such as “ARDS” (Acute Respiratory Distress Syndrome), “Normal,” and “COPD” (Chronic Obstructive Pulmonary Disease). These labels are non-limiting and exemplary only. Other implementations may output continuous values, risk probabilities, severity scores, or regression-based predictions for clinical parameters such as tidal volume, respiratory resistance, or dynamic lung compliance.

The connections between all layers-shown as directed edges represent trainable weight matrices W^{{[1]}}, W^{{[2]}}, W^{{[3]}} and corresponding bias vectors b^{{[1]}}, b^{{[2]}}, b^{{[3]}}. These are optimized during training using backpropagation and gradient-based optimization algorithms, such as stochastic gradient descent or Adam. Additional training techniques, such as dropout regularization, batch normalization, learning rate decay, and gradient clipping, may be employed to improve generalization and stability.

While FIG. 8 depicts a fully connected feedforward architecture, other non-limiting neural network topologies may include:
Convolutional layers for detecting local features and spatial dependencies
Recurrent or gated layers for capturing temporal dynamics in sequential data
Transformer blocks with self-attention mechanisms for modeling long-range dependencies
Residual or skip connections to mitigate vanishing gradient issues in deep networks

The deep learning model represented in FIG. 8 may be trained using datasets that include labeled physiological signals from both healthy individuals and those with respiratory conditions. These datasets may include waveform segments annotated by clinical experts, synthetic variations for rare event simulation, and multimodal features from wearable sensors. The model may be validated using cross-validation, test sets, or prospective clinical trial data to ensure generalizability and safety.

Once trained, the model structure shown in FIG. 8 may be deployed within the machine learning inference block 540 of FIG. 5, forming part of the real-time decision engine that continuously monitors patient breathing, classifies respiratory states, and supports early detection of deterioration or condition change. Model outputs may trigger alerts via the alert logic module, populate summary metrics in the GUI 102, or interface with therapy coordination systems such as the therapy control module. Additional downstream use cases may include updating population dashboards or closed-loop ventilator control.

In summary, the deep learning model shown in FIG. 8 provides an example of how sensor-derived feature vectors may be transformed through multiple computational layers to generate meaningful clinical predictions. The depicted configuration is illustrative only and may be adapted, scaled, or replaced with alternative architectures or data sources consistent with the disclosed system and methods.

FIG. 9 illustrates an example accuracy curve 900 of a trained artificial intelligence (AI) model, showing model accuracy as a function of learning iterations during the training phase of a deep learning-based respiratory condition recognition system. The figure represents a non-limiting example of how model performance may improve over time with exposure to training data derived from wearable physiological sensors.

The horizontal axis 902 may represent the number of learning iterations, gradient descent steps, or epochs used to train the model. In the illustrative embodiment, the iteration count extends beyond 3 million steps, reflecting a high-resolution training regime conducted over an extensive dataset. The vertical axis 904 may correspond to model accuracy, which may include any of classification accuracy, validation accuracy, cross-entropy loss inverse, or any other metric derived during supervised or semi-supervised training.

The accuracy curve 900 shown in FIG. 9 may represent the cumulative improvement in predictive accuracy over time as the model ingests more data and updates its internal weights (e.g., weights such as

$${w}_{\{ ij \}}^{\{ ( 2 ) \}} , {w}_{\{ jk \}}^{\{ ( 3 ) \}} ,$$

etc., as shown in FIG. 8). The curve may exhibit an initial rapid increase in accuracy, followed by a gradual leveling off, which may indicate convergence (906) to a locally optimal or globally optimal set of parameters. This behavior may be typical of deep learning systems trained using backpropagation and stochastic gradient descent (SGD) or its variants (e.g., Adam, RMSprop, or Adagrad).

In various example embodiments, the model depicted in FIG. 9 may be derived from architectures such as the one shown in FIG. 8 and trained using features extracted from physiological signals acquired via wearable sensors 200 (see FIG. 2), including diaphragm sensor 202 and accelerometer 207 outputs. The training dataset may include data collected from a diverse population of subjects, including both healthy individuals and those with respiratory pathologies such as obstructive or restrictive lung conditions.

The training phase reflected in FIG. 9 may be conducted offline, such as during system development or model tuning, and the resulting model may be deployed to run in real-time or near-real-time classification modes during actual patient monitoring, as described in FIG. 5. The accuracy trends shown in FIG. 9 may assist developers or clinical engineers in selecting training stopping points, assessing overfitting or underfitting, or validating generalization capability prior to deployment.

Although FIG. 9 depicts a single performance curve over time, in other embodiments multiple curves may be generated, such as training vs. validation accuracy, precision-recall tradeoffs, or confidence intervals. Additionally, accuracy curves may be generated for models trained using other input modalities (e.g., SpO₂, CO₂, posture), other neural architectures (e.g., convolutional, recurrent, attention-based), or for other classification targets (e.g., disease progression scoring, ventilation responsiveness prediction, or dyspnea severity grading).

The example accuracy curve 900 in FIG. 9 may thus serve to illustrate that the described AI model can be trained to a high level of accuracy given sufficient data and appropriate architecture, enabling its use in clinical support systems for respiratory health monitoring, therapy adjustment, or risk prediction.

A validation accuracy curve may optionally be plotted alongside the training curve, enabling analysis of model generalization. Divergence between the training accuracy curve and the validation accuracy curve may signal potential overfitting, prompting model regularization, early stopping, or adjustment of network complexity. A threshold line may be added to indicate a minimum acceptable model performance level required for clinical deployment, such as 90% accuracy or better.

A convergence indicator may optionally mark the region of the curve where learning slows and performance stabilizes, helping developers determine the optimal training endpoint. This may support efficient use of compute resources and minimize the risk of overfitting.

Although FIG. 9 shows a single composite performance curve, in other embodiments, multiple curves may be generated and plotted, including:
Training vs. validation accuracy
Cross-validation fold performance
Precision-recall curves
Receiver operating characteristic (ROC) curves
Confidence interval bands or uncertainty quantification regions

In addition, accuracy curves may be generated for alternative neural architectures (e.g., convolutional, recurrent, transformer-based), or for models targeting different outputs such as disease trajectory prediction, ventilation responsiveness estimation, or dyspnea severity scoring.

The example shown in FIG. 9 thus serves to illustrate how model performance may evolve through progressive exposure to training data, highlighting the model's capacity for learning meaningful patterns and supporting its use in a clinical respiratory monitoring pipeline. The presence of a convergence indicator 906 and a clearly defined threshold line 900 may guide developers, data scientists, and regulatory reviewers in evaluating model maturity, safety, and clinical applicability.

FIG. 10 illustrates a perspective or top-down view of an extended wearable belt design configured to enable simultaneous acquisition of multiple physiological signals relevant to respiratory, cardiac, and metabolic health monitoring. This multi-sensor assembly may be used in conjunction with the base unit 100 (see FIG. 1), or as part of an alternative integrated or modular monitoring system.

The belt strap 1000 may be configured to wrap circumferentially around the patient's torso, typically in the upper abdomen (thoracoabdominal) region just below the rib cage. The belt strap 1000 may be formed from elastic or non-elastic material and may include adjustable segments, fastening mechanisms, or sizing markers to accommodate a wide range of body types and use contexts, such as ambulatory monitoring or supine clinical positioning.

Affixed to the belt strap 1000 may be one or more sensing components. In the illustrated embodiment, the belt may include an Accelerometer Sensor 1005, a MMG sensor 1010, which may detect mechanomyographic activity corresponding to diaphragm motion. The MMG sensor 1010 may use capacitive-based vibration or strain detection to capture localized surface deformations associated with respiratory effort. These signals may correspond to tEAdi signal waveform 302 (FIG. 3) or signal amplitude surface 404 (FIG. 4), and may be used in downstream feature extraction pipelines as described in FIG. 5.

Adjacent to or integrated with the MMG component may be an ECG sensor 1020, configured to detect cardiac electrical signals from a thoracic or abdominal lead placement. The ECG sensor 1020 may support real-time detection of heart rate, arrhythmias, or signal morphology shifts associated with respiratory effort, postural changes, or autonomic stress. ECG data may also assist in phase-aligning respiratory and cardiac waveforms, enabling cardiorespiratory coupling analysis or differential filtering.

In some embodiments, a SpO₂ sensor 1030 may be integrated into the belt structure, using reflective or transmissive photoplethysmographic (PPG) techniques to estimate blood oxygen saturation. The SpO₂ sensor 1030 may be positioned along the lateral or anterior portion of the belt strap 1000, or routed to a peripheral site (e.g., finger, earlobe) via a modular extension. PPG signals from the SpO₂ sensor 1030 may be used in fusion models described in FIGS. 23 and 24, particularly for respiratory desaturation detection and modeling of oxygenation-effort mismatches during sleep, exertion, ventilation, or dialysis. The sensitivity of signals from SpO₂ sensor 1030 and diaphragm (MMG) sensor 1010 may be assessed in relation to changes in device settings or through a longitudinal comparison of the sensors' responses over time.

All sensor modules may be enclosed or mounted within a shared or segmented housing 1040, which may protect sensitive electronics and provide a unified mechanical interface for skin contact and cable retention. The housing 1040 may include internal compartments for each sensing modality and may be constructed from biocompatible materials that support long-term wear, disinfectability, and moisture management. Structural features such as gel pads, mounting clips, or heat sinks may be integrated to enhance signal quality or patient comfort.

Signal and power connections for the multiple sensors may be routed through a multi-sensor cable bundle 1050, which may converge at a single connector leading to the cable to base unit 210 (FIG. 1) or to a separate wireless transmission module. In some configurations, the multi-sensor cable bundle 1050 may include shielding or differential signal paths to reduce cross-talk between ECG, MMG, and PPG channels. Cable strain relief mechanisms and locking connectors may be used to ensure safe and durable connections during patient repositioning or mobility.

In operation, the multi-sensor wearable shown in FIG. 10 may support simultaneous acquisition of respiratory effort (via the MMG sensor 1010), cardiac status (via the ECG sensor 1020), and oxygenation (via the SpO₂ sensor 1030). These data streams may be combined in the feature extraction block 530 and analyzed by the machine learning inference block 540 (see FIG. 5) or by cloud-based algorithms described in FIG. 6.

The modular, belt-based configuration allows rapid deployment in inpatient, outpatient, or home settings, and supports integrated physiological monitoring with minimal burden on the patient. The belt may be worn under clothing and may remain in place for extended monitoring periods (e.g., overnight, post-surgical recovery, or during rehabilitation programs). In other embodiments, belt form factors may be modified to support pediatric, geriatric, or bariatric populations, or adapted to serve as part of a harness, garment, or flexible wrap.

FIG. 10 thus illustrates a multi-modal wearable sensor configuration that enhances the clinical utility of the system by enabling synchronized capture of multiple physiological parameters. This integration supports more robust machine learning inference, real-time condition classification, and early detection of events such as hypoxia, bradycardia, respiratory fatigue, or ventilator-patient asynchrony.

FIG. 11 illustrates a block diagram of a comprehensive patient monitoring system architecture that integrates multiple physiological input streams and analytical modules to support continuous, real-time respiratory and cardiac assessment. The configuration shown in FIG. 11 enables synchronized acquisition, aggregation, and trend analysis across several physiologic domains, including respiration, heart activity, and vital sign monitoring.

The system may include a respiratory input 1100, which may receive respiratory effort signals from one or more wearable diaphragm sensors 200 as shown in FIG. 2. These signals may be mechanomyography signals from the MMG sensor 1010 (FIG. 10), and posture-corrected effort readings from the accelerometer. The respiratory input 1100 may also include derived waveform features, such as respiratory rate, I:E ratio, or RSBI, as computed by the machine learning inference block 540 (FIG. 5).

In parallel, a cardiac input 1110 may receive signals from one or more ECG sensors 1020 (FIG. 10) or other electrocardiogramonitoring sources or be derived from PPG sensor data 1030 (FIG. 10). The cardiac input 1110 may support beat-to-beat interval analysis, arrhythmia detection, and heart rate variability computation, providing insight into autonomic tone and cardiopulmonary interaction. In some configurations, the cardiac input 1110 may include real-time waveform streaming or preprocessed heart rhythm labels supplied by upstream classifiers.

A vital sign module 1120 may receive and store data from both respiratory and cardiac channels, as well as from other available sources such as SpO₂ sensor 1030, temperature probes, or CO₂ sensors. The vital sign module 1120 may also track cumulative statistics, trends, and alert thresholds across all vital domains, integrating both raw signals and derived parameters.

The data aggregation processor 1130 may serve as a central integration node that synchronizes timestamps, aligns multi-sensor input streams, and resolves inconsistencies in sampling rates or data dropouts. The data aggregation processor 1130 may normalize signal units, apply calibration models, or perform sensor fusion operations to construct unified patient state representations. This processor may be implemented locally on the base unit 100, remotely in a cloud service, or across distributed compute nodes (see FIG. 6).

Aggregated data may be passed to a trend analysis engine 1140, which may generate rolling averages, identify abrupt deviations, or detect slowly evolving baseline shifts. The trend analysis engine 1140 may implement algorithms that flag clinical deterioration patterns, such as progressive hypoventilation, declining oxygenation, increased respiratory variability, or correlated changes in heart rate and effort. In some embodiments, predictive models (trained as described in FIG. 7) may be embedded within this module to generate risk scores, early warning indices, or therapy guidance cues.

Results and derived insights may be displayed or transmitted via an output interface 1150, which may correspond to the GUI 102 (FIG. 1), a clinician-facing dashboard, or a mobile caregiver interface. The output interface 1150 may include visualizations of multi-sensor data, annotated alerts, waveform overlays, and decision-support messages. Data presented via the output interface 1150 may also be logged into the electronic health record interface 640 (FIG. 6) or exported to external systems using HL7, FHIR, or proprietary data formats.

In use, the architecture shown in FIG. 11 enables integrated, multimodal physiological monitoring that reflects both short-term status and long-term patient trajectories. The system may operate continuously in acute care environments, or intermittently during rehabilitation, post-discharge monitoring, or outpatient screening. By unifying respiratory input, cardiac input, and supplemental vital sign monitoring within a single architecture, the system enhances situational awareness, facilitates early intervention, and enables closed-loop or semi-automated decision-making for a range of clinical use cases.

FIG. 12 illustrates an example of a GUI (GUI) that can be used to present real-time respiratory metrics and system status for clinician interpretation, configuration, and monitoring according to various example embodiments. The GUI may be implemented locally on the display of the base unit 100, or presented on a connected mobile device, workstation, tablet, or web-accessible interface within a clinical or home environment.

In the illustrated example, a parameter trend overlay 1260 is shown at the top of the display and may present a real-time waveform such as a tEAdi signal waveform 302 (FIG. 3), a filtered MMG signal, or any other physiologic trace derived from the wearable diaphragm sensor 200. The parameter trend overlay 1260 may be continuously updated to reflect current respiratory dynamics, waveform morphology, or processed output from signal feature extraction modules (e.g., FIG. 5).

Beneath the waveform display, a series of real-time quantitative metrics may be shown using a combination of digital values and vertical bar graphs. These may include, but are not limited to:
A respiratory rate bar 1200 configured to display current breathing frequency (in breaths per minute) alongside a historical range or threshold scale;
A tidal volume inhaled bar 1210 displaying the inspiratory tidal volume (VTi), derived from respiratory waveform integration or model-based estimation;
A tidal volume exhaled bar 1220 showing the exhaled tidal volume (VTe), which may reflect air trapping or ventilatory asymmetry;
A minute volume bar 1230 representing the calculated minute ventilation (MV), typically in liters per minute;
An I:E ratio bar 1240 displaying the inspiratory-to-expiratory duration ratio, which may support detection of restrictive or obstructive patterns;
An RSBI bar 1250 showing the rapid shallow breathing index, a non-invasive surrogate for fatigue or readiness-to-wean in ventilated or monitored patients.

Each of these bars may include color-coded histograms or shaded regions reflecting min/max boundaries, individualized baselines, clinician-set thresholds, or guideline-derived target zones. These visual indicators may assist users in quickly identifying trends, excursions, or risks without requiring deep waveform analysis.

The GUI may also include an alert indicator panel 1270, which can display hardware alerts, physiologic parameter deviations, or data integrity issues. Alerts may be issued by the alert logic module 102 (FIG. 1), and may include visual icons, text banners, auditory signals, or remote notifications depending on system configuration.

A navigation menu 1280 may be positioned along one side of the interface and may allow the user to access different display modes, review historical trends, configure patient parameters, or manage system settings. The interface may include touch-based, pointer-driven, or voice-controlled interactions, depending on the form factor of the device used.

A screen lock button 1290 may be provided to prevent accidental activation or modification of monitoring parameters. This feature may be especially useful in environments with high patient mobility, shared workstations, or during patient transport. Additional interface elements (not separately labeled in the current illustration) may include “Stop Monitoring,” session timers, patient demographics, or battery/network status indicators.

In various example embodiments, the GUI of FIG. 12 may be highly configurable, enabling the user to select which parameters are shown, adjust scaling, choose color schemes, or toggle between numerical and graphical representations. The system may also support dynamic annotation, allowing clinicians to mark events, enter observations, or synchronize with patient-reported symptoms entered via mobile interfaces.

Data presented on the GUI may originate from local inference engines (e.g., machine learning inference block 530, FIG. 5), trend summarization modules (e.g., trend analysis engine 1140, FIG. 11), or cloud-based models (cloud machine learning engine 650, FIG. 6). Outputs may be mirrored in remote dashboards, sent to the clinical decision support module 660, or integrated into the electronic health record interface 640, depending on the configuration.

In some embodiments, the GUI may also support overlay of therapy data (e.g., ventilator settings, neuromuscular stimulation events), synchronizing these with physiological readings to support clinician feedback and therapy optimization. Display elements may update in real-time, semi-real-time, or periodically, based on patient stability, bandwidth availability, or clinician preference.

Accordingly, FIG. 12 provides a non-limiting example of a GUI designed to support real-time, multi-parameter respiratory monitoring in a flexible, user-configurable format, capable of operating in hospital, outpatient, or home environments, and supporting clinician decision-making through accessible and context-rich visualization.

FIG. 13 illustrates an example of a longitudinal respiratory rate waveform chart according to various example embodiments. The figure shows how respiratory rate measurements may be visualized over an extended period—such as five weeks—in a format designed to support clinical interpretation, retrospective analysis, and trend detection.

The time axis 1300 spans a continuous monitoring period and may represent time in absolute or relative units (e.g., seconds, hours, days, or epochs). In the illustrated example, the time axis extends across approximately 4,500 units, divided into week segments demarcated by a week label band 1350, which provides a temporal context for clinicians and analysts reviewing the data.

The respiratory rate signal 1310 represents the raw or preprocessed measurement of breaths per minute (bpm), computed from input streams acquired via the wearable diaphragm sensor 200, including the diaphragm sensor 202 and/or the accelerometer. The respiratory rate signal 1310 may be derived from tEAdi waveforms, MMG data, or other mechanical or electrical indicators of respiratory cycles. The signal may be sampled continuously or at predefined intervals depending on the clinical setting or device configuration.

A trend smoothing curve 1340 is overlaid on the signal, illustrating broader trends in breathing rate over time. The trend smoothing curve 1340 may be calculated using moving averages, Gaussian filters, exponential smoothing, or low-pass digital filters, and may be updated in real time or during post-processing. This curve enables easier identification of long-term changes in respiratory rate, such as those caused by therapy response, disease progression, medication effect, or physical recovery.

The respiratory rate signal 1310 is shown in multicolored bands, corresponding to different measurement clusters or detection events. These are visualized as color-coded event segments 1330, which may help identify periods of abnormal variability, increased effort, or measurement artifact. Colors may correspond to underlying signal quality metrics, diagnostic classifications (e.g., obstructive, restrictive, normal), or machine learning predictions.

Specific dates and events may be annotated on the chart using date markers 1320, which are positioned above the signal line. These date markers 1320 may represent clinical annotations, patient-entered symptom logs, medication changes, or milestone events (e.g., onset of dyspnea, intervention administered, hospital discharge, etc.). These may be entered through the GUI 102, a mobile caregiver portal, or a clinician dashboard.

In various example embodiments, the chart shown in FIG. 13 may be generated by the trend analysis engine 1140 (FIG. 11) and visualized via the output interface 1150, which may be locally displayed or shared across care teams via the cloud machine learning engine 650 or clinical decision support module 660 (FIG. 6). The visualization may assist in identifying clinically significant respiratory trends, such as:
Gradual increases in respiratory rate due to infection, fluid overload, or metabolic acidosis;
Day-night rhythm disruptions due to sleep-related breathing disorders;
Sudden spikes suggesting pain, panic, or obstruction;
Post-intervention changes confirming treatment efficacy or decompensation.

In some configurations, additional channels may be layered onto the chart, such as oxygen saturation, heart rate, or symptom scores, enabling multi-dimensional interpretation. Clinicians may interact with the visualization to zoom into a specific week or date range, annotate observations, or export waveform segments to the electronic health record interface 640 (FIG. 6).

FIG. 13 provides a non-limiting example of how respiratory rate data may be transformed into an informative, longitudinal trend chart, supporting clinical decision-making, patient monitoring, early deterioration detection, and machine learning model validation. The use of dynamic visualization, smoothing overlays, and time-aligned annotations enables clinicians and researchers to interpret complex time-series data in a meaningful and actionable way.

FIG. 14 illustrates an example of a tidal volume waveform chart that can be used to visualize multi-week respiratory monitoring data according to various example embodiments. The illustrated chart shows changes in tidal volume (measured in milliliters) over time and may assist clinicians in assessing ventilatory adequacy, recovery trends, or the effects of intervention across various care settings.

The time axis 1400 spans a multi-week period, which in this example is divided into week label bands 1450 labeled Week 1 through Week 5. These bands help orient the clinician or reviewer when analyzing data trends. The time units may be represented in hours, minutes, days, or other sampling intervals, depending on system configuration and visualization scale.

The tidal volume signal 1410 represents measured or estimated values of inspired or expired tidal volume (VTi or VTe) per breath. This data may be acquired from the wearable diaphragm sensor 200 using signals from the diaphragm sensor 202, accelerometer, or in combination with derived values from machine learning inference block 540 (FIG. 5). In some embodiments, volume estimates may be generated using model-based inference from tEAdi, MMG, or impedance sensors, rather than spirometry alone.

A trend smoothing curve 1440 is overlaid on the tidal volume signal 1410, helping to identify long-term increases, decreases, or stabilization of tidal volume. This smoothed curve may be calculated using running average filters, low-pass filters, or advanced modeling techniques such as LOESS, Kalman filtering, or Gaussian process regression. The smoothing window may be adjustable by the user or automatically tuned based on signal variability.

Different color-coded event segments 1430 are used to visually delineate clusters of measurements or to highlight changes in signal character, physiological state, or sensor quality. These segments may correspond to different physical activity states, patient posture changes, signal confidence levels, or automated classifications from machine learning models (e.g., “normal,” “at risk,” “unstable”). The system may adjust segment coloring in real-time or as part of retrospective review, supporting pattern recognition and contextual interpretation.

Annotated date markers 1420 are positioned above the waveform, indicating days on which key clinical or environmental events occurred. These may include interventions, symptom onset, medication administration, or sensor configuration changes. The date markers 1420 may be manually entered via the GUI 102, automatically triggered by changes in respiratory status, or imported from external data streams such as patient self-reports or electronic medical records.

In various embodiments, the tidal volume signal 1410 shown in FIG. 14 may be used to derive one or more secondary parameters, such as:
Minute volume (via integration with respiratory rate);
Dynamic compliance (in conjunction with pressure measurements);
Air-trapping index (by comparing VTi and VTe over multiple breaths);
Tidal volume variability (TVV) as a marker of respiratory irregularity or neuromuscular fatigue.

The chart may be rendered by the trend analysis engine 1140 (FIG. 11) and visualized on the output interface 1150, accessible via bedside systems, mobile clinician application, or centralized dashboards. Longitudinal visualization of tidal volume signal 1410 may assist clinicians in evaluating patient status post-extubation, during home oxygen therapy, or throughout rehabilitation following acute respiratory events.

Although FIG. 14 depicts a single continuous signal over five weeks, in other example embodiments, data may be sampled intermittently (e.g., once per hour), binned by sleep/wake cycles, or visualized across different dimensions (e.g., posture, therapy type, or sensor position). The system may also provide interactive zoom, date filtering, or anomaly flagging tools to enhance usability.

Accordingly, FIG. 14 provides a non-limiting example of how tidal volume data may be displayed over time to support monitoring, analysis, and decision-making, using a signal display framework that is adaptable to different patient types, clinical environments, and monitoring goals.

FIG. 15 illustrates an example of a minute volume waveform chart that can be used to monitor respiratory function across multiple weeks in various example embodiments. This chart shows temporal fluctuations in minute ventilation (MV), typically measured in liters per minute (L/min), which may reflect both the depth and rate of breathing. The visual structure of the chart supports detection of clinically significant patterns, including gradual decompensation, acute instability, or recovery following intervention.

The time axis 1500 spans a continuous period of respiratory monitoring, segmented into week label bands 1550 (e.g., Week 1 through Week 5). These week labels help contextualize the longitudinal data and align it with clinical timelines, symptom logs, or therapy sessions.

The minute volume signal 1510 represents a real-time or aggregated value calculated from the product of respiratory rate and tidal volume over a rolling window. The data may be derived from signals acquired by the wearable diaphragm sensor 200, including the diaphragm sensor 202 and accelerometer, and may be estimated using inference models described in machine learning inference block 540 (FIG. 5).

The minute volume signal 1510 may be color-segmented into different color-coded event segments 1530, which may represent physiological state changes, signal confidence levels, or classifier output groups (e.g., “normal,” “increased effort,” “shallow pattern,” etc.). These colored segments may be generated by the trend analysis engine 1140 (FIG. 11) based on changes in minute volume, co-occurrence with heart rate or SpO₂ shifts, or clinician-defined thresholds.

A trend smoothing curve 1540 is shown overlaid on the waveform, generated using rolling statistical methods such as exponential moving average, Kalman filters, or spline-based smoothing. The trend smoothing curve 1540 helps clinicians interpret broad directional shifts or confirm the stability of respiratory effort over time.

Date markers 1520 positioned above the signal may annotate specific clinical events or milestones, such as medication changes, therapy interventions, or symptom reports. These date markers 1520 may be manually entered via the GUI 102, or automatically annotated based on event detection algorithms, wearable data trends, or remote caregiver input.

In some embodiments, additional derived metrics may be calculated from the minute volume signal 1510, including:
MV variability: standard deviation or coefficient of variation over time;
MV thresholds: detection of hypoventilation (<4 L/min) or hyperventilation (>12 L/min) periods;
Recovery curves: change in MV slope post-intervention or over rehabilitation phases;
Ventilatory reserve ratio: comparing predicted vs. observed MV during activity.

The system may display the minute volume waveform chart on the output interface 1150, integrated bedside systems, mobile clinician application, or centralized dashboards. Clinicians may use the visualization to track minute ventilation during post-extubation monitoring, sleep, physical therapy, or medication titration.

Although FIG. 15 shows a continuous MV trace across five weeks, other formats may display shorter snapshots, synchronized multi-parameter overlays (e.g., with respiratory rate and SpO₂), or bin data into trend score categories for simplified dashboards.

Accordingly, FIG. 15 provides a non-limiting example of how minute volume data may be visualized over time to support respiratory monitoring, clinical analysis, and machine-assisted decision-making. This figure reinforces the system's capacity to derive, display, and interpret core respiratory parameters with sufficient fidelity for deployment in both high-acuity and home-care settings.

FIG. 16 illustrates an example of an inspiratory-to-expiratory ratio (I:E ratio) waveform chart that may be used to assess respiratory pattern dynamics over time according to various example embodiments. This visualization enables clinicians and analysts to monitor changes in the temporal structure of breathing cycles, providing insight into breathing effort, airflow obstruction, restrictive physiology, or therapy effects.

The time axis 1600 represents the elapsed monitoring duration and may be segmented into week label bands 1650 (Week 1 through Week 5, as shown), which may help contextualize observed trends within longer-term therapy, recovery, or disease progression timelines.

The I:E ratio signal 1610 may represent the breath-by-breath ratio of inspiratory time to expiratory time. This ratio may be computed in real time from signal landmarks derived from the wearable diaphragm sensor 200, including the diaphragm sensor element 202, accelerometer, and optionally, a SpO₂ sensor 1030 or airflow measurement proxy. In some embodiments, the I:E ratio may be calculated using machine learning inference, using waveform segmentation and breath pattern classification performed by the machine learning inference block 540 (FIG. 5).

A trend smoothing curve 1640 overlays the I:E ratio signal 1610, offering a visual summary of how the patient's baseline breathing pattern may be evolving. For example, a prolonged inspiratory phase (I:E>2:1) may suggest restrictive disease or stress-related breathing, while a prolonged expiratory phase (I:E<1:2) may be indicative of obstructive lung disease. The trend smoothing curve 1640 may be updated dynamically using digital filtering, low-pass smoothing, or model-based estimation.

The I:E ratio signal 1610 is also segmented into color-coded event segments 1630, with each color representing a shift in respiratory state or confidence level. These segments may be assigned by the trend analysis engine 1140 (FIG. 11) based on signal thresholds, waveform morphology, or automated classifications (e.g., “stable,” “restrictive,” “postural shift,” etc.).

Date markers 1620 are displayed across the top of the waveform and may be used to annotate key time points such as intervention delivery, reported symptom onset, therapy adjustment, or system configuration changes. The date markers 1620 may be entered manually via the GUI 102, automatically populated based on system logs, or linked to patient-reported data via mobile caregiver platforms.

In various example embodiments, I:E ratio may be interpreted in isolation or alongside other respiratory parameters shown in FIGS. 13-15, such as:
Elevated I:E combined with declining tidal volume→fatigue or neuromuscular compromise
I:E flattening (approaching 1:1)→stress breathing or upper airway obstruction
I:E drift during sleep vs. wake cycles→detection of sleep-disordered breathing
Postural I:E shifts correlated with accelerometer data→supine breathing analysis

This visualization may be rendered within the output interface 1150 and made accessible through bedside systems, mobile clinician application, or centralized dashboards. The system may allow for zooming, annotation, overlaying additional parameters, or exporting of I:E ratio signal 1610 segments for external analysis or training of new models (e.g., FIG. 7).

Although the illustrated waveform shows a single, continuous I:E ratio signal 1610, alternative embodiments may bin the data into hourly summaries, present percentile ranges, or generate alerts when the ratio exceeds or drops below predefined thresholds set by clinicians or automatically tuned by the system. Such threshold excursions may be routed to the alert logic module 102 to trigger interventions or notifications.

Accordingly, FIG. 16 provides a non-limiting example of how I:E ratio data may be visualized over time to support analysis of respiratory mechanics, identification of obstructive or restrictive breathing trends, and machine-assisted clinical decision-making. The figure supports claims involving both time-series respiratory parameter computation and longitudinal trend assessment.

FIG. 17 illustrates an example of a rapid shallow breathing index (RSBI) waveform chart that can be used to monitor patient respiratory effort and fatigue trends over a multi-week period, according to various example embodiments. RSBI is a widely recognized clinical indicator used to assess weaning readiness from mechanical ventilation and detect early signs of respiratory compromise.

The time axis 1700 spans a multi-week monitoring window, segmented by week label bands 1750 indicating five sequential weeks (Week 1 through Week 5). These bands help contextualize long-term changes and facilitate correlation with clinical interventions or symptom reports.

The RSBI signal 1710 represents the breath-by-breath or smoothed ratio of respiratory rate to tidal volume (RR/Vt), expressed in breaths per minute per liter (bpm/L). The signal may be derived from sensor data acquired by the wearable diaphragm sensor 200, including the diaphragm sensor 202 and accelerometer, and may be computed using inference models running within the machine learning inference block 540 (FIG. 5).

A trend smoothing curve 1740 overlays the RSBI signal 1710, providing a rolling average or model-estimated baseline of respiratory effort. This curve may help clinicians quickly identify transitions into high-RSBI zones (e.g., >100 bpm/L), which may indicate risk of fatigue, weaning failure, or compromised ventilatory capacity.

The RSBI signal 1710 is displayed using segmented color bands, or color-coded event segments 1730, which may highlight regions of interest including abnormal RSBI elevations, recovery zones, or uncertain/confounded data. These segments may be generated by the trend analysis engine 1140 (FIG. 11), based on user-defined thresholds, confidence metrics, or anomaly detection algorithms.

Date markers 1720 may be placed across the upper region of the graph to indicate specific events, such as symptom onset, intubation/extubation, medication changes, therapy adjustments, or caregiver annotations. These date markers 1720 may be added manually via the GUI 102, or automatically through system event logs, remote clinician dashboards (FIG. 22), or patient-reported updates via mobile interfaces (FIG. 29).

In various embodiments, the RSBI values visualized in RSBI signal 1710 may be used for:
Predicting weaning success in ventilated patients (e.g., RSBI<105 bpm/L as a weaning criterion);
Detecting fatigue onset in spontaneously breathing patients during high-demand periods;
Tracking recovery after respiratory failure or neuromuscular weakening episodes;
Triggering alerts via the alert logic module 102 when RSBI exceeds defined thresholds.

The waveform shown in FIG. 17 may be displayed on the output interface 1150, and integrated with real-time monitoring dashboards, historical trend views, or decision-support systems. In some cases, the system may cross-correlate the RSBI signal 1710 with other time-series metrics—such as tidal volume signal 1410 (FIG. 14), respiratory rate signal 1310 (FIG. 13), and minute volume signal 1510 (FIG. 15)—to refine interpretations and reduce false positives in RSBI-triggered alerts.

While FIG. 17 illustrates a continuous RSBI trace across five weeks, alternative configurations may present this data in heatmap format, bar chart summaries, or regression-based trend projections. The system may also support interactive chart features such as zooming, date filtering, anomaly flagging, or threshold adjustment by the clinician.

Accordingly, FIG. 17 provides a non-limiting example of how RSBI may be visualized over time to support respiratory monitoring, clinical assessment, and machine-assisted decision-making. This figure reinforces the system's capabilities in managing weaning decisions, tracking fatigue, and enhancing early detection of respiratory decompensation in a variety of patient care contexts.

FIG. 18 illustrates an example layout diagram of a haptic feedback module embedded in a wearable sensor according to various example embodiments. The wearable sensor 200 may also provide haptic feedback to the patient or user. This feedback may be triggered when certain thresholds are exceeded, such as during respiratory distress or inadequate breathing. For example, the system may send a vibration signal through the wearable sensor to alert the patient or caregiver about a detected respiratory anomaly. This feature is particularly useful in settings where visual or auditory signals may be missed, such as in noisy environments or for patients with impaired vision. The haptic feedback module may provide local, non-visual, tactile alerts to the user or patient wearing the device, and may be used in clinical, home, or ambulatory settings. In some embodiments, the haptic module may serve as a local feedback mechanism complementing visual indicators (e.g., GUI displays or LEDs) and remote notifications delivered via dashboards or mobile interfaces.

The core mechanical structure shown in FIG. 18 is a wearable sensor housing 1800, which may correspond to the same housing shown in FIG. 2 and may house respiratory sensing components such as the diaphragm sensor 202, accelerometer, or signal conditioning circuitry. In the present configuration, the wearable sensor housing 1800 is modified to incorporate the haptic system internally.

A haptic motor 1810 may be embedded within the wearable sensor housing 1800, and may be configured to generate vibrational pulses, buzz patterns, or mechanical taps detectable by the wearer. The haptic motor 1810 may be a coin motor, linear resonant actuator (LRA), eccentric rotating mass (ERM), or piezoelectric element, and may be coupled directly to the interior housing wall or suspended via flexible mounts to ensure skin-conductive feedback.

The vibration controller 1820 may regulate the amplitude, frequency, duration, and pattern of haptic signals generated by the haptic motor 1810. In various example embodiments, the controller may be implemented using low-power embedded microcontrollers or as part of the device's main signal processing unit. The vibration controller 1820 may support preset or programmable feedback patterns (e.g., single pulse, double buzz, escalating tap), depending on the alert type or interaction logic.

A trigger logic module 1830 may receive input from other system components and determine when and how to activate the haptic motor. Trigger conditions may include, for example:
Threshold breaches in respiratory parameters (e.g., RSBI, I:E ratio, tidal volume);
Calibration confirmation or failure (see FIG. 18);
Disconnection of the sensor from the base unit 100;
System alerts routed from the alert logic module (FIG. 1);
Confirmation of patient-initiated actions (e.g., symptom log acknowledgment or breath-hold instruction feedback).

In some embodiments, the trigger logic module 1830 may operate in coordination with machine learning classification outputs (e.g., FIG. 5 or 6), using risk scores or anomaly detections as input conditions for haptic signaling.

The module may also include an alert status LED 1840, mounted externally on the wearable sensor housing 1800, to provide visual confirmation of an alert, calibration status, or system mode. The alert status LED 1840 may blink or glow continuously during different operational states and may be color-coded (e.g., green for monitoring, yellow for warning, red for error).

An optional user interface feedback button 1850 may allow the user to acknowledge an alert, silence the haptic signal, or initiate a user-initiated calibration or data mark. The user interface feedback button 1850 may be implemented as a mechanical pushbutton, capacitive touch sensor, or soft-sealed interface designed for hygienic use and long wear-time.

The haptic feedback module shown in FIG. 18 may operate autonomously or as part of a broader multi-modal alerting and interaction system. In some embodiments, haptic feedback may be triggered even if the user is not actively viewing the GUI 102 (FIG. 12), providing a fallback mechanism for urgent alerts. This may be especially useful for high-risk patients, ambulatory users, or patients with visual impairments.

Although FIG. 18 depicts a haptic module embedded within the wearable sensor housing 1800, alternative embodiments may include belt-mounted, patch-mounted, or wireless haptic actuators. The system may support adjustable sensitivity, customizable alert types, and secure over-the-air update protocols for modifying feedback logic remotely.

Accordingly, FIG. 18 provides a non-limiting example of a haptic feedback module embedded within a wearable respiratory sensor system (200, FIG. 2), enabling tactile communication between the system and the user in support of safety, usability, and clinical responsiveness across a wide range of use environments.

FIG. 19 illustrates an example block diagram of a cloud-based infrastructure for routing respiratory monitoring data and integrating patient information with electronic health record (EHR) systems, according to various example embodiments. The cloud infrastructure shown may support secure, scalable, and interoperable data exchange across care teams, institutions, and analytic systems in both real-time and batch processing contexts.

The system may include a remote monitoring server 1900, which can receive, aggregate, and manage incoming physiological data from one or more base units 100 or mobile-connected wearable diaphragm sensors 200. The remote monitoring server 1900 may support continuous telemetry, periodic sync, or store-and-forward configurations based on patient context, bandwidth, or usage policy.

Incoming data may be passed through a data router 1910, which is configured to direct each data stream to the appropriate processing or storage destination. The data router 1910 may operate using rule-based logic, machine learning-driven prioritization, or real-time load balancing. For example, high-risk alerts may be routed to clinician dashboards immediately, while lower-priority trend data may be queued for later review.

A secure API gateway 1920 may enforce encryption, authentication, and access control policies. The secure API gateway 1920 may validate credentials for external applications (e.g., hospital portals, caregiver apps, or research tools) and control access to protected patient data. This gateway may implement industry-standard protocols such as OAuth2.0, TLS 1.3, FHIR RESTful APIs, and mutual authentication.

To support clinical integration, an electronic health record (EHR) sync module 1930 may translate device-generated data into standardized formats (e.g., HL7 v2, FHIR resources) and push updates to hospital record systems. The EHR sync module 1930 may map machine learning-derived respiratory parameters—such as RSBI, MV, or I:E ratio (FIGS. 13-17)—to structured fields in the patient's longitudinal record.

In parallel, data may be mirrored to a backup storage node 1940, which may maintain immutable, versioned, or encrypted archives for regulatory, forensic, or analytics purposes. The backup storage node 1940 may support tiered storage models (e.g., hot, warm, cold) depending on retrieval frequency and clinical relevance. In some embodiments, it may also facilitate audit trails or support FDA 21 CFR Part 11 compliance.

An analytics engine 1950 may provide advanced data processing capabilities, including signal reanalysis, population-level trend extraction, AI model retraining (FIG. 7), or research cohort aggregation. The analytics engine 1950 may be accessed by authorized researchers or clinicians, and may provide real-time dashboards, predictive insights, or retrospective performance summaries.

This cloud infrastructure may operate in concert with the cloud machine learning engine 650 (FIG. 6), and may serve as a backbone for distributed decision support across facilities, telehealth networks, or national health systems. All modules in FIG. 19 may scale horizontally to support increasing numbers of connected patients, wearable units, or third-party applications.

Although the architecture in FIG. 19 is shown in discrete modular blocks, alternative implementations may consolidate components, distribute them across cloud regions, or deploy hybrid edge/cloud configurations. The infrastructure may also support asynchronous data workflows, customizable alerts, failover redundancy, and compliance with data localization regulations.

Accordingly, FIG. 19 provides a non-limiting example of a cloud-based architecture for routing physiologic data and synchronizing with clinical systems, enabling longitudinal respiratory monitoring, collaborative decision-making, and secure data interoperability across a wide variety of care environments.

FIG. 20 illustrates an example flowchart of a therapy override protocol involving clinician confirmation according to various example embodiments. This protocol may be used in systems where respiratory therapy is influenced by machine learning predictions or automated control logic (see FIG. 5 and FIG. 6), and serves to ensure that clinicians retain supervisory authority over therapeutic changes or system-driven recommendations.

The process may begin with a patient trigger input 2000, which may originate from multiple sources, including:
Manual interaction by the patient, such as pressing a user interface feedback button 1850 (FIG. 18) or interacting with a mobile interface;
Physiological thresholds being crossed (e.g., a sudden spike in RSBI signal 1710, or persistent tachypnea);
Machine learning model outputs (e.g., from machine learning inference block 540, FIG. 5) indicating the need for therapy escalation, ventilation mode change, or stimulation activation.

Once a triggering condition is met, the system routes the event to a clinician review node 2010, which presents a summary of relevant parameters, trends, and model interpretations through the GUI 102 (FIG. 12) or other authorized portals. This may include trend overlays, alert timelines, and structured therapy suggestions (e.g., “increase inspiratory support,” “pause stimulation,” or “recalibrate thresholds”).

The clinician is then prompted to perform an override confirmation 2020, which may involve:
Accepting the system's suggested therapy adjustment;
Modifying the proposed settings (e.g., changing pressure levels or disabling escalation);
Rejecting the adjustment entirely and returning to a prior baseline.

This step may require password entry, digital signature, or biometric authentication to ensure authorized control and traceability. In some embodiments, audit logging is performed simultaneously by the electronic health record interface 640 (FIG. 6) or EHR sync module 1930 (FIG. 19).

Before the action is implemented, a safety logic gate 2030 evaluates the proposed override against pre-defined clinical boundaries. For instance, even if approved by a clinician, therapy settings that exceed safe limits (e.g., respiratory rate thresholds, minute volume constraints, or contraindicated stimulation levels) may be automatically rejected or require secondary authorization. This gate may be governed by hard-coded limits, guidelines (e.g., ARDSNet), or adaptive risk models informed by population data.

If the override is approved and passes safety review, the system proceeds to an alert output 2040, which may:
Notify care teams through visual or auditory alerts (see alert logic module 102, FIG. 1);
Update therapy devices or ventilator interfaces;
Document the action and rationale in clinical records;
Adjust machine learning inference behavior to incorporate supervised override examples.

In real-time implementations, the therapy override protocol shown in FIG. 20 may also support asynchronous modes—for example, when a remote clinician is reviewing a flagged condition but has not yet responded. In such cases, the system may suspend action, maintain current therapy settings, and re-evaluate the override request on a recurring schedule or after a configured timeout period.

The protocol shown may be adapted for different environments:
In ICU settings, override confirmation may be mandatory for all model-driven recommendations;
In outpatient or telemonitoring systems, override may occur asynchronously with escalation logic (e.g., notify the clinician and delay change for 24 hours if no rejection is received);
In closed-loop control scenarios, override actions may also trigger a re-weighting of inference confidence or update model tuning.

Accordingly, FIG. 20 provides a non-limiting example of a therapy override protocol that combines real-time physiological monitoring, machine learning decision-making, and clinician authority, supporting safe, transparent, and accountable integration of AI into respiratory care workflows.

FIG. 21 illustrates an example diagram of sensor fusion logic that integrates carbon dioxide (CO₂) and temperature data into a respiratory model, according to various example embodiments. In some embodiments, the sensor fusion system may dynamically adjust the weight given to each sensor input based on context, such as patient posture, activity level, or the specific type of respiratory issue being monitored. For example, during sleep, the system may reduce the influence of motion-based sensors like accelerometers, while giving greater weight to the diaphragm sensor for detecting subtle breathing patterns. The real-time data processing engine adjusts the thresholds based on this multi-sensor feedback, ensuring more accurate, patient-specific predictions and alerting. The diagram in FIG. 21 shows how environmental and metabolic indicators can be combined with existing respiratory signals to enhance the precision, robustness, and context-awareness of respiratory parameter estimation and clinical inference.

The CO₂ sensor input 2100 may represent data acquired from a capnography sensor, transcutaneous CO₂ monitor, or integrated nasal/oral cannula outfitted with an end-tidal CO₂ sensor. In various example embodiments, the CO₂ sensor input 2100 may provide a waveform corresponding to the capnogram (CO₂ concentration over time) and/or discrete values such as end-tidal CO₂ (EtCO₂), partial pressure of carbon dioxide, peak CO₂, and phase slope metrics. The sensor may be wired into the base unit 100, wirelessly connected, or embedded in the wearable diaphragm sensor 200.

The temperature sensor input 2410 may originate from a thermistor, thermocouple, or infrared thermopile integrated into the respiratory path, housing, or adjacent skin contact surface. The temperature sensor input 2410 may provide continuous or event-based updates of local or core temperature, and may support respiration-phase tracking (e.g., detecting exhaled air as temperature rises), compensating for thermal drift in mechanical sensors, or correlating with circadian or febrile effects on respiration.

Both signals are routed to a data normalization unit 2120, which may preprocess each input to a common scale, unit system, or temporal resolution. This may include resampling, interpolation, filtering, or z-score transformation. The data normalization unit 2120 ensures that the input streams are compatible with downstream fusion models regardless of originating sensor resolution, noise level, or sampling latency.

The preprocessed signals are then passed to a fusion logic core 2130, which may implement various fusion strategies, including:
Weighted averaging based on real-time signal quality;
Feature-level concatenation for input to a unified classifier or regressor;
Decision-level fusion where parallel models contribute individual predictions that are merged via voting, averaging, or meta-learning (e.g., stacked generalization);
Conditional fusion where temperature or CO₂ input is used as a gating signal or context vector for modulating inference (e.g., attention weights or residuals in a deep learning model, FIG. 8).

In some embodiments, the fusion logic core 2130 may also be used to refine core respiratory signals like RSBI, tidal volume, or I:E ratio when the primary signal quality (e.g., from the diaphragm sensor 202) is degraded. For instance, rising CO₂ and falling temperature variance may trigger recalibration, signal substitution, or increased inference uncertainty estimates.

The output of the fusion process is the enhanced respiratory parameter output 2140, which may include one or more of the following:
Smoothed or confidence-weighted respiratory rate, tidal volume, or minute volume;
Adjusted baseline thresholds (e.g., to account for thermally induced respiration changes);
Risk-adjusted RSBI predictions;
Early warning signals derived from divergence between CO₂ and mechanical effort;
Multimodal composite indicators for decision support modules.

These enhanced outputs may be transmitted to the machine learning inference block 540 (FIG. 5), logged into the electronic health record interface 640 (FIG. 6), or visualized via the output interface 1150. They may also trigger or modify alert behavior through the alert logic module, enabling CO₂- and temperature-modulated alert tuning (e.g., reduce alert frequency in febrile conditions or suppress false positives during transient hypoventilation).

Although FIG. 21 shows CO₂ and temperature as the fusion targets, additional sensor modalities may be added, such as SpO₂, ECG, or motion signals. Fusion models may be retrained periodically using the model training processor 730 (FIG. 7) and performance evaluated via the validation accuracy curve 906 (FIG. 9).

Accordingly, FIG. 21 provides a non-limiting example of a sensor fusion logic diagram for incorporating auxiliary physiologic data into a respiratory model, enhancing signal quality, interpretability, and robustness across varying clinical and ambulatory use conditions.

FIG. 22 illustrates an example exploded view of a wearable sensor configured to acquire diaphragmatic motion and effort signals, according to various example embodiments. This view expands upon the wearable designs shown in FIG. 2 and FIG. 10, and details the physical structure and internal components of the device, which supports accurate signal acquisition, patient comfort, and long-duration use.

At the base of the assembly is the belt strap 2200, which may wrap around the patient's torso to hold the wearable device in place. The belt strap 2200 may be adjustable, elastic or non-elastic, and include buckles, Velcro®, or other fastening mechanisms described in prior figures. The strap provides anchoring for the sensor assembly and ensures appropriate positioning of the diaphragm-sensing components.

Secured to the belt is the outer housing 2210, which encloses the internal sensing hardware and protects it from environmental exposure, impact, or patient movement. The outer housing 2210 may be formed from lightweight, biocompatible plastics, flexible polymers, or multilayer composites, and may be shaped to conform to the abdominal surface or intercostal spaces.

Integrated with the housing is the diaphragm sensor 2220, which may include a stretchable capacitive sensor, piezoresistive strip, flexible strain gauge, or a micromechanical system configured to detect diaphragmatic motion. The diaphragm sensor 2220 may be configured to capture tEAdi, MMG, or other diaphragm-related motion signatures in more than one dimension and may feed data into the system's core signal processing pipeline (e.g., FIG. 5).

Adjacent to or integrated with the diaphragm sensor is an accelerometer 2230, which may detect gross body motion, postural orientation, or respiration-related movement artifacts. The accelerometer 2230 may provide multi-axis (e.g., 3D) inertial data used for signal correction, motion artifact rejection, and respiratory phase detection (as shown in FIG. 3, FIG. 20). In some embodiments, this sensor also supports patient fall detection, sleep posture classification, or cough detection.

Both sensors connect to a signal conditioning board 2240, which may include analog front-end circuitry, analog-to-digital conversion, signal amplification, impedance matching, and filtering components. The signal conditioning board 2240 may also include a microcontroller or local processor to manage data acquisition, calibration (FIG. 18), power management, and wireless communication functions.

Beneath the housing, a skin contact interface pad 2250 may provide soft, secure coupling to the patient's body. The skin contact interface pad 2250 may be made of gel-based adhesive, breathable fabric, medical-grade silicone, or hydrophilic foam. It may be removable, disposable, or cleanable, depending on clinical requirements. This interface supports stable signal coupling, reduces motion artifacts, and ensures comfort during long-term wear.

In some embodiments, the entire sensor module shown in FIG. 22 may be modular and detachable, allowing rapid sensor replacement without removing the full belt assembly. The system may also support different sensor cartridges for different use cases (e.g., pediatric vs. adult, supine vs. ambulatory, mechanical ventilation vs. spontaneous breathing).

Power, data, and ground lines may be routed internally to the signal conditioning board 2240, which may communicate with the base unit 100 via a wired cable (FIG. 1) or a wireless transmitter embedded in the housing. The module may also include additional sensors or components 2260, such as temperature sensors (FIG. 24), vibration motors (FIG. 18), or haptic feedback circuits.

Accordingly, FIG. 22 provides a non-limiting exploded view of a wearable respiratory sensor assembly, detailing how internal components—including the diaphragm sensor, accelerometer, and signal processing board—are integrated within a patient-facing device designed for robust, comfortable, and accurate monitoring. This figure supports claims involving wearable construction, signal acquisition, modularity, and patient safety.

FIG. 23 illustrates an example logic diagram for detecting mismatches between respiratory effort and oxygen saturation during dialysis according to various example embodiments. This figure highlights how the system integrates multi-sensor data to detect physiologically significant divergences—such as cases where the patient demonstrates high respiratory effort but fails to maintain adequate oxygen saturation—during dialysis, ultrafiltration, related fluid management procedures, or between dialysis sessions.

The system may receive input from two core sources. First, the MMG signal input 2600 may originate from the MMG sensor 1010 embedded in the wearable diaphragm sensor 200 (see FIG. 10 and FIG. 22), and may reflect mechanomyographic signatures of respiratory muscle activity, including breath effort, cycle rate, and muscular fatigue. These signals may be filtered and extracted via the feature extraction block 530 and interpreted through the machine learning inference block 540 (FIG. 5).

Second, the SpO₂ input 2310 may be obtained from the SpO₂ sensor 1030, also embedded in the wearable or connected peripherally. This signal may be processed using the system's sensor fusion logic (FIG. 23), and may provide continuous or trend-based readings of oxygen saturation percentage (% SpO₂) during dialysis sessions.

The two signal streams are passed to a mismatch detection engine 2320, which may apply temporal alignment, signal quality scoring, and conditional logic to determine when significant mismatches occur. For example:
Scenario A: High MMG activity (indicating increased breathing effort) with declining or stagnant SpO₂ levels;
Scenario B: Low or absent MMG signal (suggesting shallow or failed inspiration) with slow desaturation;
Scenario C: Cyclical divergence—e.g., desaturation occurring post-breath-hold or during hypotensive episodes.

The mismatch detection engine 2320 may operate using rules-based logic (e.g., thresholds and timing windows), statistical methods (e.g., correlation coefficients, time-delay cross-correlation), or learned models trained on mismatched event data (e.g., see FIG. 7).

The output is routed to an alert threshold logic 2330 module, which evaluates whether the detected mismatch exceeds a configurable alert threshold based on patient condition, dialysis protocol, or clinician configuration. Thresholds may be time-based (e.g., mismatch persisting for >20 seconds), magnitude-based (e.g., effort index increase>30% with SpO₂ decrease >3%), or probabilistic (e.g., likelihood of mismatch exceeding 95% confidence). This module may trigger alerts via the alert logic module 102 (FIG. 1) or update risk displays in the clinician dashboard.

Simultaneously, the system may interface with a dialysis machine sync 2340, which allows alignment with ultrafiltration phases, fluid removal cycles, or session timelines. The dialysis machine sync 2340 may be implemented via hardware connection (e.g., RS-232, Bluetooth), API integration with modern machines, or manually entered schedules. Synchronization allows the system to distinguish between effort-oxygen mismatches due to ventilatory insufficiency vs. hemodynamic events (e.g., intradialytic hypotension, pulmonary congestion).

The detection and alerting logic in FIG. 23 may operate continuously or intermittently throughout a dialysis session, and alerts may be presented via the bedside systems, mobile clinician application, or centralized dashboards for collaborative care.

In some embodiments, mismatch detections may be annotated onto longitudinal parameter charts (e.g., RSBI signal 1710, FIG. 17; minute volume signal 1510, FIG. 15), or used to trigger recalibration of thresholds or re-weighting of decision logic. The system may also store mismatch events for inclusion in EHRs via the EHR sync module 1930, along with clinician notes and therapy adjustments.

Accordingly, FIG. 23 provides a non-limiting example of a logic diagram for detecting respiratory effort and oxygenation mismatches during dialysis, using integrated sensor data, machine learning, and context-aware alerting to enhance patient safety and care quality during high-risk treatment periods.

FIG. 24 illustrates a block diagram showing integration between a wearable respiratory monitoring system and a ventilator in a closed-loop control configuration, according to various example embodiments. The system is designed to optimize respiratory support using patient-specific signals, AI-driven inference, and bidirectional communication with therapeutic devices, while preserving clinician oversight and system safety.

At the center of the system is the patient 2400, connected physiologically to both the ventilator 2410 and the base unit 100. The patient's real-time respiratory effort signals—such as MMG waveform amplitude, RSBI, and inspiratory timing—are acquired by the wearable diaphragm sensor 200 (as described in FIGS. 2, 10, and 22), and processed within the base unit 100.

The RSBI computation unit 2420 calculates the rapid shallow breathing index (RSBI) and may also analyze other metrics including I:E ratio, respiratory rate (RR), tidal volume, and minute volume (MV). These metrics are derived from signals produced by the diaphragm sensor element 202, MMG sensor 1010, and accelerometer, and optionally augmented by data from CO₂ and SpO₂ inputs (see FIG. 21).

Results are evaluated by the decision threshold logic 2430, which determines whether changes in patient condition warrant ventilator adjustment. Thresholds may be configured manually or derived dynamically using patient-specific tuning or population-informed models such as those shown in adaptive threshold curve. In some embodiments, these thresholds are further adjusted by contextual variables including posture, fatigue trajectory, or recent therapy history.

If a change is indicated, the system uses a control signal generator 2440 to formulate a parameter modification, which is passed to the ventilator via a ventilator parameter interface 2450. The connection occurs over a bidirectional communication link 2460, enabling the monitor to both read current ventilator settings and issue real-time control instructions. This integration supports dynamic updates to pressure support, backup respiratory rate, or timing synchronization settings based on continuous patient condition.

In embodiments intended for therapy-assist or closed-loop ventilator control, the system may include safety features aligned with medical device regulatory frameworks such as ISO 13485 (quality management), IEC 62304 (medical device software lifecycle), or FDA Class II software-controlled devices. For example, software executing the therapy control logic (e.g., control signal generator 2440, decision threshold logic 2430, and ventilator interface module 2450) may include watchdog timers, fail-safe defaults, and interlocks requiring explicit clinician override. Safety-critical subsystems may incorporate dual-channel verification, data integrity checks, and user prompts to confirm parameter changes before transmitting commands to connected ventilators. All adjustments may be logged with time-stamped metadata for traceability, auditability, and clinical signoff. These features enable the invention to be implemented in regulated environments while ensuring patient safety and enabling real-time therapy adaptation. For example, where deployed in safety-critical or regulated environments, the systems and methods disclosed herein may comply with relevant standards including IEC 62304 (medical device software lifecycle), ISO 14971 (risk management), and FDA guidance for software as a medical device (SaMD). Closed-loop configurations may include interlocks, dual-channel safety logic, or clinician override requirements.

In various embodiments, the base unit 100 may connect to commercial ventilators via a standardized ventilator interface module (FIG. 1), which may support serial protocols (e.g., RS-232), proprietary APIs (e.g., GE, Philips, or Hamilton ventilator interfaces), or cloud-based control via FHIR-compatible endpoints. Integration may be performed by configuring command mapping tables within the control signal generator 2440, which translate system outputs (e.g., RSBI threshold crossings) into ventilator-specific parameter adjustment commands. In scenarios requiring FDA or MDR compliance, these connections may include built-in safety interlocks and clinician override prompts to ensure regulatory alignment and traceability.

The monitoring feedback loop then tracks post-adjustment response metrics—such as RSBI reduction, synchrony restoration, or decreased effort—and confirms whether the system should retain the new setting, revert, or escalate to clinician review.

In some embodiments, where direct device control is unavailable or not enabled, the system may operate in a recommendation-only fallback mode, wherein control instructions are presented as suggestions within the clinician dashboard, mobile interface, or GUI (FIG. 12), rather than enacted directly. These suggestions may include confidence scores, rationale summaries, and acknowledgment logging to support human-in-the-loop oversight and compliance auditing.

In various example configurations, if signal quality deteriorates or inference confidence falls below a predefined threshold, the system may default to passive monitoring mode, suspend further adjustments, and issue a notification for clinician review. This behavior ensures safe fallback under conditions of uncertainty or sensor degradation.

The clinical optimization outputs 2470 represent the system's intended outcomes and include:
Optimizing the weaning experience, by helping clinicians assess readiness and reduce respiratory burden;
Optimizing pressure and volume support, ensuring individualized, responsive therapy;
Improving patient-ventilator synchrony, minimizing asynchronous cycles and associated discomfort or fatigue.
These clinical benefits are supported by core parameter monitoring, including:
I:E ratio and respiratory rate
RSBI and breathing effort (e.g., MMG amplitude)
Minute volume and respiratory rate, used to assess ventilation adequacy and control dynamics

The entire framework may interface with other system components described throughout this disclosure, including:
The machine learning inference block 540 (FIG. 5);
Safety logic from the therapy override protocol (FIG. 20);
Longitudinal trend visualization and alerting (FIGS. 11, 13-17);
Threshold and decision support personalization (FIG. 6);
Logging and audit for system validation.

FIG. 25 illustrates an example block diagram showing system logic paths for therapy-assist mode and monitoring-only mode, according to various example embodiments. This figure describes how the respiratory monitoring system may be configured to operate flexibly in one of two primary modes—monitoring-only or therapy-assist—and how transitions between these modes may be triggered, validated, or constrained by system rules, patient context, or clinician input.

At the beginning of the decision flow is the sensor input node 2500, which collects incoming signals from the wearable diaphragm sensor 200, including real-time data from the MMG diaphragm sensor 202 (1010), accelerometer (2230), and optionally additional sources such as SpO₂ sensor 1030 (FIG. 10) or CO₂ input 2100 (FIG. 21). These signals may be passed through the system's core signal processing chain (see FIG. 5) and fused using the fusion logic core 2130 (FIG. 21).

In monitoring-only mode, data is routed through the monitoring-only logic path 2510, which may include trend analysis (FIG. 11), alert generation (FIG. 1), visual display on clinical dashboards, mobile views, or integration into health records (FIG. 19). In this mode, the system provides observation, annotation, and risk detection functionality without attempting to modify therapy settings or influence ventilator operation.

In therapy-assist mode, data is routed to the therapy-assist logic path 2520, which includes inference-driven actions such as:
Computation of actionable respiratory metrics (e.g., RSBI 1710, MV 1510, I:E 1610);
Execution of closed-loop logic (FIG. 24) via the control signal generator 2440 and ventilator parameter interface 2450;
Delivery of therapy recommendations via therapy suggestion panel on clinician dashboard or override requests via the clinician review node 2010 (FIG. 20);
Integration with therapy timing, delivery confirmation, and feedback validation via monitoring feedback loop (FIG. 24).

To determine which mode is active or should be engaged, the system invokes a mode transition condition evaluator 2530. This module checks for conditions such as:
User-set configuration preferences (e.g., ICU bedside unit set to assist mode);
Device permissions or compatibility (e.g., connected ventilator via interface 2450);
Risk thresholds or trigger events (e.g., RSBI spike, post-weaning trial failure);
Explicit clinician command or policy-driven defaults (e.g., home setting defaults to monitor-only).

In some embodiments, this transition evaluator may apply logic based on population-informed thresholds, or system override rules (FIG. 20). When transition criteria are met, the system may shift modes automatically, queue the transition for clinician approval, or recommend the transition visually via output interface 1150 or the GUI 102.

Final outputs from either path are handled by an output routing interface 2540, which may direct data or control instructions to one or more destinations, including:
On-screen clinician interfaces (FIG. 12);
Mobile alerts;
Device commands (e.g., ventilator adjustment via FIG. 24);
Logging modules and EHR systems (FIG. 19).

The system may log all decisions and transitions to maintain an auditable history and to support continuous learning through model retraining workflows (see training dataset storage 720, FIG. 7).

Although FIG. 25 shows a binary decision architecture, alternative embodiments may include hybrid modes (e.g., suggest-only mode), layered permissions (e.g., therapy assist with human signoff), or adaptive confidence thresholds that govern transitions between passive and active engagement.

Accordingly, FIG. 25 provides a non-limiting example of dual-mode system logic supporting both passive respiratory monitoring and therapy-assist operation, with built-in intelligence for mode transition, safety, clinical authorization, and real-time patient context adaptation.

FIG. 26 illustrates a flowchart showing an example of a control loop for automated ventilator adjustment based on the calculated rapid shallow breathing index (RSBI), according to various example embodiments. This flowchart outlines the real-time, inference-based logic used to derive therapy control signals based on patient-specific respiratory effort indicators, while supporting safe clinical supervision and adaptive control.

The process begins with an RSBI input node 2600, which receives raw or derived signals from the wearable diaphragm sensor 200, including those from the diaphragm sensor 202 and accelerometer. These signals are used to compute respiratory rate (RR) and tidal volume (VT), which together yield the rapid shallow breathing index (RSBI=RR/VT), a clinically validated predictor of weaning readiness and ventilatory efficiency (see RSBI signal 1710, FIG. 17).

The computation engine 2610 performs RSBI calculation using breath-by-breath or windowed data from the system's real-time signal processing pipeline (see FIG. 5). The computation engine 2610 may apply filtering, confidence scoring, and artifact rejection, and may optionally weight RR and VT based on posture, sensor quality, or patient-specific baselines.

The RSBI value is then passed to a threshold evaluation module 2620, which compares the current value against static or adaptive thresholds. These thresholds may include:
A clinically accepted baseline (e.g., RSBI<105 for weaning readiness);
Dynamic thresholds derived from adaptive threshold curve;
Context-aware bounds based on posture, time of day, or recent effort trends.

If the RSBI crosses one of these thresholds—e.g., exceeds the fatigue threshold or drops below the support-reduction trigger—the system advances to the adjustment logic block 2630. This block determines whether and how ventilator parameters should be modified, based on predefined mappings or model-driven suggestions.

Examples of adjustments include:
Increasing pressure support or inspiratory time if RSBI is rising;
Decreasing ventilator backup rate during recovery phases;
Triggering a clinician alert to confirm a proposed setting change;
Logging a therapy escalation event for subsequent review.

Once the appropriate adjustment is determined, the ventilator command output 2640 issues the corresponding instruction to the ventilator parameter interface 2450 (FIG. 24). This output may be transmitted via a secure connection (wired or wireless) or shown to a clinician via GUI (FIG. 12) for final confirmation before activation.

In some embodiments, the ventilator command output 2640 may also activate a monitoring feedback loop (FIG. 24), which evaluates patient response over time to validate or reverse the intervention. Additionally, data from this control cycle may be archived and used for future model retraining (FIG. 7) or input into therapy escalation scoring protocols.

Alternative implementations of this loop may support:
Multivariable input logic (e.g., RSBI+SpO₂+MV);
Confidence-weighted adjustments with built-in safety gates;
Integration with asynchronous caregiver systems via mobile alerts;
Fall-back to manual assist mode based on the dual-mode logic of FIG. 25.

Accordingly, FIG. 26 provides a non-limiting example of an RSBI-based control loop that continuously monitors breathing efficiency, evaluates against adaptive thresholds, and generates context-aware ventilator control outputs, forming a core feedback mechanism within a broader AI-driven respiratory support system.

FIG. 27 illustrates a diagram of a machine learning engine configured to perform patient-specific threshold adaptation based on historical respiratory data and population-level models, according to various example embodiments. This figure supports the system's ability to tailor its classification and alert thresholds to each individual, accounting for variability in physiology, disease status, therapy response, and clinical outcome history.

The process begins with a historical respiratory data input 2700, which may include previously collected values for key respiratory parameters such as RSBI, minute volume, tidal volume, I:E ratio, and respiratory rate, as shown in FIGS. 13-17. These data may originate from prior monitoring sessions using the wearable diaphragm sensor 200, and may include both raw waveform segments and derived parameters extracted via the feature extraction block 530 (FIG. 5).

The system accesses a baseline model repository 2710, which stores one or more reference models that reflect generalized behavior across the monitored population. These may include:
Normative thresholds (e.g., RSBI<105);
Disease-specific parameter expectations (e.g., higher RR tolerance in restrictive disease);
Population-segmented classifiers derived during model training (FIG. 7);
Empirically tuned models validated via the cloud machine learning engine 650 (FIG. 6).

At the same time, a clinical outcomes input 2720 provides ground truth data about previous episodes, including:
Whether prior alerts corresponded to real clinical deterioration (e.g., respiratory failure);
Success or failure of ventilator weaning attempts;
Patient responses to therapy escalation (e.g., whether RSBI dropped after pressure support increase);
Symptoms reported or documented (e.g., via manual input control).

The adaptive logic module 2730 receives the three inputs and applies supervised, semi-supervised, or reinforcement learning techniques to update the system's decision thresholds or inference rules. This may include:
Gradient descent or Bayesian updates on model weights;
Confidence-based reweighting of alert parameters;
Customization of baseline thresholds for RSBI, MV, or I:E ratio for each individual;
Incorporation of comorbid factors, postural response variability or therapy history.

The adaptive logic module 2730 may run periodically (e.g., overnight), upon new clinical event resolution, or continuously in real-time systems. The module may also quantify confidence or risk in any recalibration, and flag potentially unstable adjustments for clinician review via the dashboard or override logic (FIG. 20).

The output is a set of updated threshold outputs 2740, which are passed downstream to influence alerting logic (FIG. 1), control decisions, and visualization overlays. These thresholds may be:
Stored in the patient's profile for longitudinal use;
Displayed in GUI interfaces to promote transparency;
Audited and versioned for compliance and traceability (via EHR sync module 1930, FIG. 19).

In some embodiments, updated threshold outputs 2740 may also be used as labels or features in further rounds of model retraining (FIG. 7), supporting continual system improvement across the monitored population.

For example, a patient with COPD may initially present with an RSBI baseline of 80 during early recovery. Over a 72-hour period, if the system detects a consistent reduction in tidal volume alongside increasing inspiratory time and rising fatigue signals, the adaptive logic module 2730 may shift the RSBI escalation threshold from 105 to 95 to trigger earlier intervention. This adjustment may be based on population-trained rules, personalized outcome history, or clinician-approved adaptations logged via the override module (FIG. 20). The updated threshold 2740 may then be used to alter alert logic, escalation scoring, and therapy suggestion behavior.

Although shown as a discrete engine, the logic in FIG. 27 may be embedded within the cloud-based infrastructure 1900 (FIG. 19), run locally on edge hardware, or distributed across nodes for federated learning deployments. Patient-specific adaptations may also be rolled back, versioned, or reset upon clinical command or adverse signal detection.

Accordingly, FIG. 27 provides a non-limiting example of how a machine learning engine may combine individual patient data, generalized model knowledge, and outcome history to refine respiratory decision thresholds and improve monitoring accuracy, personalization, and clinical safety across diverse deployment scenarios.

FIG. 28 illustrates a flowchart showing a therapy escalation decision protocol based on multi-parameter trend analysis and severity scoring, according to various example embodiments. This protocol enables the system to synthesize complex respiratory monitoring data into actionable, risk-weighted recommendations for therapy adjustment, escalation, or clinician review.

The process begins at the multi-parameter input node 2800, which receives respiratory signals and derived parameters from the real-time monitoring system. These may include, without limitation:
RSBI signal 1710, minute volume signal 1510, and tidal volume signal 1410 from FIGS. 17-14;
Trend analytics from the trend analysis engine 1140 (FIG. 11);
Posture-adjusted baselines, patient-specific thresholds, and mismatch markers.

The signals are passed to the deviation analyzer 2810, which evaluates the current state of each parameter relative to:
Personalized thresholds derived from the adaptive logic module 2730 (FIG. 27);
Recent trends or slopes from trend smoothing curves (FIGS. 13-17);
Static limits embedded in system configuration or population-based guidelines.

The deviation analyzer 2810 may assess both instantaneous and time-aggregated deviation, factoring in the duration, magnitude, directionality, and parameter interplay (e.g., a simultaneous rise in RSBI and fall in MV may be more concerning than either alone).

The output of this analysis feeds the severity score generator 2820, which assigns a numeric or categorical severity rating. This score may be computed using:
Rule-based weighted scoring (e.g., breath effort>80% of max+MV drop>30%=“High Severity”);
Probabilistic models (e.g., likelihood of deterioration in next 4 hours);
Learned classifiers trained on historical outcome data (via training dataset storage 720, FIG. 7).

Each severity score may be adjusted based on posture, patient history, or time of day. Confidence levels and signal quality metrics may also be factored into the calculation.

The result passes into the escalation decision logic 2830, which determines whether a therapy change should be recommended, suggested with clinician review, deferred, or rejected. Escalation logic may be configured for:
Automatic action under closed-loop protocols (FIG. 24);
Clinician-in-the-loop suggestions requiring confirmation (FIG. 20);
Asynchronous mobile alerts delivered to caregivers;
Documentation-only logging for audit and longitudinal modeling.

If escalation is warranted, the system issues a therapy change recommendation output 2840, which may include:
A proposed ventilator setting change (e.g., increase pressure support);
Activation of a therapy (e.g., neuromuscular stimulation or oxygen delivery);
A recommendation to transition modes (see mode transition evaluator 2530, FIG. 25);
A suggestion to engage the therapy override protocol.

The therapy change recommendation output 2840 may appear in the clinician dashboard, be annotated on a time-aligned input overlay, or routed to a decision review queue within the cloud machine learning engine 650 (FIG. 6). Final actions may be contingent on feedback from clinicians, caregivers, or the system's safety layer.

In alternate implementations, the logic of FIG. 28 may run continuously, on-demand (e.g., after an alert or input), or on a recurring schedule. Outputs may be versioned, risk-scored, and tracked for longitudinal therapy outcome modeling or retraining.

Accordingly, FIG. 28 provides a non-limiting example of a therapy escalation decision flow that uses multi-parameter respiratory trends and severity scoring to generate context-aware therapy change recommendations, closing the loop on real-time respiratory monitoring and adaptive, safe, and explainable clinical intervention.

### 1. Overview of System Assembly

In various example embodiments, the respiratory monitoring system may include a wearable sensor configured to detect diaphragmatic activity, a base unit for processing and interface, and associated modules for signal acquisition, transmission, storage, and display. This section describes how a person of ordinary skill in the art may construct and assemble the system components.

### 1.1 Wearable Sensor Assembly

The wearable portion of the system may be assembled beginning with the belt strap 2200 (FIG. 22), which may be fabricated from flexible, breathable, and hypoallergenic materials suitable for extended patient wear. The belt strap 2200 may incorporate adjustable fasteners such as a buckle 212 (FIG. 2) or hook-and-loop systems to accommodate a wide range of torso sizes. Elastic or non-elastic materials may be selected based on the clinical use case, patient mobility, and the degree of mechanical coupling desired.

Secured to the belt strap 2200 is the outer housing 2210 (FIG. 22), which may be injection-molded or fabricated from biocompatible polymer composites. The outer housing 2210 may be designed to protect and support internal electronic components while conforming to the contours of the patient's upper abdomen.

Enclosed within the outer housing 2210, the diaphragm sensor element 2220 may be a capacitive strain sensor, stretch sensor, MMG pickup, or any other mechanism suitable for detecting diaphragmatic contraction and mechanical effort in more than one dimension. This element may be positioned in a mechanically stable, body-facing orientation, may be supported by a skin contact interface pad 2250. The skin contact interface pad 2250 may be formed from a gel, foam, or silicone material and may be replaceable or disinfectable for repeated use.

An accelerometer 2230 may be co-located within the same housing, affixed to a signal conditioning board 2240 that also supports analog-to-digital conversion, microcontroller logic, and signal buffering. The accelerometer 2230, may be mounted on the signal conditioning board 2240 and used to capture tri-axial motion data. In some embodiments, a haptic motor 1810 (FIG. 18) and vibration controller 1820 may also be embedded to provide tactile feedback to the wearer.

The completed sensor module, including all internal components, may then be securely closed and sealed within the outer housing 2210. Depending on system configuration, the unit may be equipped with a cable port to support a cable 122, 208 (FIG. 1, FIG. 2) for power and data, or may include an onboard battery and wireless transmission circuit.

### 1.2 Base Unit Assembly

The system may further include a base unit 100 (FIG. 1), which may be configured as a portable, cart-mounted, or bedside device. The base unit 100 may house a processor/GUI 102, which receives and processes signal data from the wearable sensor. Internally, the base unit 100 may include a machine learning module, a wireless transmitter, a cloud interface module, and an alert logic module-all which may be part of the processor/GUI 102. In therapy-assist configurations, the base unit 100 may also include a therapy control module and a ventilator interface module, also part of the processor/GUI 102.

Externally, the base unit 100 may include a GUI 102 for displaying real-time waveforms, status indicators, and interactive controls (FIG. 12). The housing may also include features such as a power supply bracket 114, cord management bracket 116, cart handle 110, and lockable wheels 120 to support use in clinical settings.

In certain implementations, the sensor cable 122 from the wearable unit may connect to the base unit 100 via a lockable connector 124, 214 to prevent accidental detachment during patient movement. Alternatively, systems equipped with wireless transmission capability may eliminate the physical cable entirely.

### 1.3 System Integration and Additional Components

The assembled system may further include interfaces for connecting to cloud infrastructure (FIG. 6, FIG. 19), electronic health record (EHR) systems (FIG. 19), or mobile caregiver devices, using secure communication protocols, application programming interfaces (APIs), or hardware links. The system may also include a battery backup, environmental enclosure, or network redundancy module (not shown) in configurations designed for continuous outpatient use.

In some embodiments, the system architecture may be modular, allowing components to be added, removed, or substituted depending on clinical context or patient-specific needs. For example, in lightweight monitoring configurations, the system may operate with only the diaphragm sensor 2220 and base unit 100, omitting secondary sensors such as SpO₂, CO₂, or temperature inputs. Conversely, expanded configurations may incorporate additional inputs such as electrocardiogramaracic impedance, speech pattern detection, or ambient environmental sensors (e.g., air quality, humidity). Each module may communicate with the processor 102 or cloud interface module using standardized protocols, and may be enabled or disabled via software configuration. This flexibility allows the system to be deployed in a range of care settings—from hospital ICU to remote outpatient environments—while still supporting core signal interpretation, trend analysis, and decision support functions.

Accordingly, a person of skill in the art would recognize that the invention may be made using commercially available microcontrollers, wearable-grade sensors, plastic enclosures, and cloud infrastructure components, assembled using routine techniques in electronics fabrication, device integration, and health device compliance, and may be adapted across a wide range of patient populations and care environments without departing from the scope of the invention.

### 2. Sensor Signal Acquisition and Calibration

In various example embodiments, the system is configured to continuously or intermittently acquire physiological signals associated with diaphragmatic effort, respiratory mechanics, and contextual variables such as posture and motion. The signal acquisition process may include mechanical coupling of the sensor assembly to the patient, real-time data capture, and execution of a calibration routine to verify signal integrity and enable downstream analysis.

2.1 Signal Acquisition from Wearable Components

During operation, the wearable diaphragm sensor 200 (see FIG. 2, FIG. 10, FIG. 22) is positioned around the patient's upper abdomen using the belt strap 2200 and affixed to the skin via the skin contact interface pad 2250 or may be placed over patient's clothing. This ensures sufficient mechanical coupling to detect subtle diaphragmatic displacement, particularly during inspiration and expiration cycles.

The diaphragm sensor element 2220, housed within the outer housing 2210, may operate as a capacitive, electroactive polymer-based sensor configured to detect displacement, stretch, or vibration signatures of diaphragmatic contraction in more than one direction of motion. This signal may represent a mechanomyographic (MMG) component, an electrical impedance shift, or other motion-correlated value.

In parallel, the accelerometer 2230 may generate tri-axial motion data that characterizes body orientation, postural change, gross movement, and high-frequency respiratory oscillations. These motion signals may be used to detect motion artifacts, confirm breath phase timing, or adjust inference confidence scores.

The outputs from the diaphragm sensor 2220 and accelerometer 2230 are routed through the signal conditioning board 2240, which performs analog filtering, signal amplification, and analog-to-digital conversion. The digitized signals are then transmitted to the base unit 100, either via a cable 210 or over a secure wireless link through the wireless transmitter 107.

In multi-sensor configurations, additional signals such as SpO₂ input 2110 and CO₂ input 2100 (see FIG. 21) may be collected from secondary sensors and routed into the same processing stream. These signals may be time-synchronized using the data aggregation processor 1130 (FIG. 11) to allow multimodal fusion.

In other example embodiments, the wearable system may incorporate or interface with additional sensor modalities including impedance-based respiratory sensors, electrocardiography (ECG) electrodes, nasal airflow sensors, or chest wall expansion belts. These may be used independently or in conjunction with the diaphragm sensor to provide multi-channel input to the machine learning engine.

### 3. Base Unit Configuration

The base unit 100 forms the processing, control, and communication hub of the respiratory monitoring system. In various example embodiments, the base unit 100 (see FIG. 1) may be configured to operate as a bedside module, mobile cart-mounted system, wall-mounted unit, or compact desktop processor. It integrates the processing hardware, machine learning engine, alerting system, user interface, and communication interfaces necessary to support both real-time signal interpretation and integration with external systems such as ventilators, electronic health records, or cloud-based dashboards.

### 3.1 Core Processing Modules

At the heart of the base unit is a processor/GUI 102, which receives incoming signal data from the wearable diaphragm sensor 200 via a wired cable 122 or wireless interface using the wireless transmitter. The processor/GUI 102 manages data acquisition, buffering, and pre-inference processing, including data formatting, time alignment, and communication with downstream modules.

A part of the processor/GUI is a machine learning module, which executes one or more pre-trained models to classify respiratory patterns, derive parameters such as RSBI, I:E ratio, tidal volume, minute volume, and detect anomalies or condition shifts. The machine learning module may operate locally using embedded firmware or software logic, or may delegate inference tasks to cloud-based models via the cloud interface module (see FIG. 6 and FIG. 19).

The base unit may include a therapy control module, which enables integration with closed-loop therapy systems. This module may initiate ventilator setpoint changes or signal therapy transitions based on inferences drawn from live respiratory data or threshold crossing events. In therapy-assist configurations, the therapy control module may also interact with the ventilator interface module to transmit commands to external ventilators or therapy delivery systems, depending on patient status, confidence scores, and escalation protocols.

A dedicated alert logic module monitors outputs from the ML engine and threshold evaluators (see FIG. 5). This module handles local and remote notification, prioritization, and integration with the GUI 102 and clinician-facing dashboards. The alert logic module may be configured to implement multilevel alerts (e.g., warning, urgent, critical).

### 3.2 GUI and User Controls

The GUI 102 may be implemented as a capacitive touchscreen display mounted directly on the base unit 100 housing (FIG. 12). The GUI presents a combination of waveform plots, bar graphs, trend indicators, and control elements. In various implementations, the GUI may include:
Live waveform displays of respiratory parameters (e.g., tidal volume, RR, RSBI);
Color-coded bar graphs showing parameter severity (e.g., FIG. 12);
Trend overlays aligned with clinician-entered events (e.g., FIGS. 13-17);
Configuration menus for patient ID, signal settings, and connectivity;
Alert controls for acknowledging, silencing, or escalating system-generated warnings.

A capacitive touch button 108 (FIG. 1) or equivalent user control element may allow quick tactile interaction with the system, such as acknowledgment of alerts, mode switching, or initiating recalibration of the wearable sensor.

### 3.3 Housing, Mobility, and Accessories

In hospital or mobile care settings, the base unit 100 may be mounted to a medical-grade mobile cart assembly, which may include:
A cart handle 110 for maneuverability;
A cart base 118 with lockable wheels 120 to maintain position during monitoring;
A power supply bracket 114 to hold batteries or an AC adapter;
A cord management bracket 116 to store excess cable 122 and reduce clutter.

The system housing may also include a sealed enclosure for sterile environments, EMI shielding, external USB or Ethernet ports for diagnostic access, and antimicrobial surface materials in long-term care use cases.

### 3.4 Communications and Connectivity

The base unit may connect to external systems using the cloud interface module 150, which may include Wi-Fi, cellular, or Ethernet capabilities, depending on deployment environment. The cloud interface module 150 enables:
Continuous or scheduled uploads to the remote monitoring server 1900 (FIG. 19);
API-based communication with EHR systems via the EHR sync module 1930;
Integration with mobile caregiver tools and dashboards.

In sum, the base unit 100 serves as the intelligent processing center of the respiratory monitoring system, integrating signal interpretation, clinical interface, safety logic, and communication control into a single, adaptable platform. It enables both passive observation and active therapy support, and may be configured across multiple form factors and deployment settings without departing from the scope of the invention.

### 4. Signal Processing and Parameter Derivation

Once physiological signals are acquired and transmitted to the base unit 100, the system begins a multi-stage digital processing pipeline to extract clinically meaningful respiratory parameters, detect abnormalities, and support real-time decision-making. This process is performed through modular software and hardware logic embedded in the system's processor and inference modules, and may operate locally or via cloud-connected infrastructure.

### 4.1 Preprocessing and Signal Conditioning

Raw signals from the diaphragm sensor element 2220 and accelerometer 2230 (FIG. 25), transmitted via the cable 208 or wireless transmitter 107, are first received and temporarily buffered by the processor/GUI 102 (FIG. 1). These digitized signals are passed through a signal acquisition block 510 and a preprocessing block 520 (see FIG. 5), which may include:
Low-pass and band-pass filtering to isolate respiratory-frequency bands;
Noise reduction algorithms for motion artifact suppression;
Temporal alignment across multi-modal signals (e.g., MMG, SpO₂, temperature, accelerometer);
Baseline normalization and dynamic range compression;
Optional signal validation logic, e.g., posture-based rejection filters (FIG. 20).

Preprocessed signals are prepared for parameter extraction and machine learning interpretation.

### 4.2 Feature Extraction and Parameter Computation

Within the feature extraction block 530 (FIG. 5), the system computes key respiratory features from the cleaned input waveforms. These features may be derived directly from signal morphology or indirectly from signal timing, frequency, or amplitude patterns. Examples include:
Breath rate (cycles per minute) derived from peak detection in MMG or accelerometer traces;
Tidal volume estimate derived from signal amplitude or derived effort waveform;
I:E ratio (FIG. 16) derived from inspiratory and expiratory time segmentation;
RSBI (FIG. 17) calculated as the ratio of respiratory rate to tidal volume (RR/VT);
Minute volume (MV) (FIG. 15) estimated as the product of RR×VT;
Rapid Shallow Breathing Index tracking and trend smoothing;
Cough, breath-hold, or apnea event detection via waveform classification.

In some embodiments, this feature extraction step includes vectorization of the signal for machine learning model input (see FIG. 8), or transformation into spectral, frequency-domain, or wavelet representations for enhanced classification accuracy.

### 4.3 Machine Learning Inference and Classification

The extracted features may be passed to the machine learning inference block 540 (FIG. 5), which may execute one or more classifiers trained on labeled respiratory condition datasets (see FIG. 7). These models may be implemented as:
Feedforward neural networks (FIG. 8);
Decision trees, SVMs, or ensemble models;
Sequence-based classifiers (e.g., RNNs) for breath-by-breath trends;
Attention-based or transformer-style architectures.

The models may be trained to detect or classify:
Obstructive vs. restrictive respiratory mechanics;
Dyspnea or fatigue onset;
Cough clusters, apnea patterns, or post-exertional desaturation;
Ventilator-patient asynchrony;
RSBI crossing escalation or de-escalation thresholds.

Thresholds applied during this inference stage may be static or dynamically adapted via the adaptive logic, using inputs from the historical respiratory data and a baseline model.

### 4.4 Multimodal Sensor Fusion

To improve specificity and robustness, the system may apply multimodal fusion logic within the sensor fusion logic core 2130 (FIG. 21). This core receives inputs such as:
CO₂ sensor input 2100 (e.g., end-tidal CO₂, pCO2 sensor);
SpO₂/PPG input 2310 (oxygen saturation);
Temperature sensor input 2110

The system may apply weighted averaging, context-aware signal gating, or late fusion strategies to generate enhanced respiratory parameter estimates (e.g., RSBI adjusted for postural hypoventilation). Fusion logic may also help resolve edge cases such as respiratory effort with paradoxical desaturation (see mismatch logic in FIG. 23).

### 4.5 Trend Generation and Alert Logic

Derived parameters may be streamed in real time to the trend analysis engine 1140 (FIG. 11), which calculates rolling averages, deltas, slopes, and risk scores over configurable time windows. Key outputs include:
Parameter deviation markers (e.g., RSBI increase>20% from baseline);
Cyclical trend oscillation detectors (e.g., Cheyne-Stokes respiration);
Fatigue risk classifiers (e.g., trending effort with decreasing tidal volume);
Severity scoring to drive escalation.

The alert logic module 102 (FIG. 1) evaluates these trend outputs against configured thresholds to generate alerts. These alerts may be:
Routed to GUI 102 or clinician dashboard;
Pushed to mobile caregiver interface;
Used to trigger control logic in closed-loop mode;
Logged with timestamp and metadata for audit and EHR sync (FIG. 19).

Alerts may also be tagged with context and confidence (e.g., “High-confidence alert: RSBI>140, SpO₂<91%, increasing fatigue trend”).

Together, this processing pipeline enables robust, adaptive respiratory monitoring that transforms raw sensor signals into clinically actionable parameters and model outputs, enabling both passive observation and active decision support across inpatient, outpatient, and home care environments.

### 5. Machine Learning Model Deployment and Training

The respiratory monitoring system described herein may incorporate one or more machine learning (ML) models configured to analyze physiological signals, classify respiratory conditions, generate parameter estimates, and adapt thresholds over time. These models may be deployed locally on the base unit 100, remotely via the cloud interface module, or in a distributed hybrid configuration. In some embodiments, the machine learning module may be configured as a modular plug-in or containerized software block, allowing substitution, retraining, or real-time switching between multiple models based on patient condition, care setting, or computational availability. This modular architecture may support clinician-selected inference profiles, automatic fallback to rule-based logic, or cross-institution model sharing using federated learning protocols. This section describes how a person of ordinary skill in the art may train and deploy such models to support real-time inference, personalization, and closed-loop respiratory control.

### 5.1 Training Dataset Generation

As shown in FIG. 7, the model development process may begin with a training dataset 720, which may include labeled physiological signal data captured using the wearable diaphragm sensor 200, including MMG signals, accelerometry, SpO₂, CO₂, and temperature input. Data may be drawn from:
Healthy and symptomatic individuals;
Controlled testing environments (e.g., post-surgical monitoring, pulmonary rehabilitation);
Ambulatory and home monitoring populations;
Synthetic or augmented data (e.g., noise injection, waveform warping, time-stretching).

The dataset may also incorporate contextual labels, such as patient posture, ventilator settings, oxygen delivery status, and clinician-confirmed episodes of respiratory decompensation or fatigue.
Training labels may be generated from:
Manual expert annotation;
EHR-confirmed events;
Outcomes recorded via clinical outcomes input;
Physiological triggers (e.g., RSBI thresholds, desaturation events).

In additional example embodiments, the system may incorporate additional input modalities such as facial EMG, speech acoustic patterns, or ambient air quality sensors to further refine respiratory status classification or alerting logic. These signals may be fused using deep structured models, such as multi-modal transformers, to improve robustness under noisy or ambiguous conditions.

### 5.2 Model Architecture and Training Process

Using the training data, a model may be trained via the training algorithm (FIG. 7), which may implement any of the following architectures:
Feedforward neural networks (e.g., multi-layer perceptrons, as shown in FIG. 8);
Convolutional networks for time-series pattern recognition;
Recurrent or gated models (e.g., LSTM, GRU) for breath-to-breath context modeling;
Transformer-based architectures for long-range temporal dependency tracking;
Ensemble or hybrid models for combining multimodal inputs.

During training, inputs may be preprocessed into fixed-length feature vectors or continuous waveforms, and may be passed through multiple hidden layers with dropout, batch normalization, or adaptive activation functions. Models may be trained using supervised learning (e.g., cross-entropy loss) or reinforcement learning (e.g., feedback from outcome signals), and may be validated using cross-validation, test-holdout partitions, or clinician-reviewed accuracy assessments.

The output of the training process is a trained AI predictive model 750, which may be versioned, performance-tested, and packaged for deployment to local and/or remote inference nodes.

### 5.3 Deployment and Real-Time Inference

Trained models may be deployed into the machine learning inference block 540 (FIG. 5) within the base unit 100, where they may receive real-time feature vectors derived from the feature extraction block 530. Inference outputs may include:
Predicted respiratory parameters (e.g., RR, VT, RSBI, MV,I:E);
Classification outputs (e.g., normal, restrictive, obstructive);
Confidence scores and anomaly flags;
Trend predictions or risk escalation scores.

In some embodiments, the trained model and associated inference logic may be stored on a non-transitory computer-readable medium, such as flash memory, RAM, ROM, magnetic disk, optical disk, or any other persistent or reprogrammable storage device operable within the base unit 100 or in connection with a cloud server.

In further embodiments, ML model outputs may include patient-specific risk scores for events such as respiratory fatigue, COPD hospitalization, or ventilator weaning failure. These scores may be presented alongside real-time parameters and used to inform clinical triage, discharge planning, or treatment escalation.

Inference results may be visualized on the GUI 102 (FIG. 12), the clinician dashboard, or the mobile caregiver, and may drive decisions rendered by the alert logic module, therapy control module, or control signal generator.

The model's accuracy and performance may be validated against real-world data streams using the Model Training Processor 730 (FIG. 7), which may compare predicted outcomes with clinician input, patient response, and feedback from the monitoring feedback loop.

### 5.4 Personalization and Adaptive Thresholds

Once deployed, ML models may continue to adapt and improve using real-time and historical patient data. In some embodiments, the machine learning model may be updated using rolling datasets collected during system operation. Updates may be performed locally or via the cloud machine learning engine 650, and may use batch retraining, transfer learning, or federated aggregation to preserve performance across heterogeneous patient populations.

As shown in FIG. 27, the adaptive logic module 2730 may recalibrate parameter thresholds based on:
Patient-specific baseline drift or recovery patterns;
Cross-patient comparisons using cohort models;
Clinician override behavior (FIG. 20);
Observed therapy response to prior escalations.

Threshold updates may be delivered via the updated threshold output 2740, logged into the electronic health record interface 640 (FIG. 6), or re-injected into the system's classifier or alert logic to drive more accurate and context-aware decisions.

In federated or cloud-based deployments, anonymized and de-identified patient data may also be used to periodically retrain or fine-tune population-level models, ensuring that inference remains relevant as the system is deployed across new clinical settings, patient populations, or use cases.

Together, these procedures support a broad range of claims directed to intelligent respiratory parameter derivation, adaptive alert generation, personalized inference, and continuous machine learning-driven improvement of wearable monitoring systems for inpatient, outpatient, or home use.

### 6. User Interfaces and Interaction

The respiratory monitoring system may include a suite of user interfaces tailored for clinical, mobile, and supervisory use. These interfaces enable users to view respiratory trends, respond to alerts, annotate patient data, approve or override therapy recommendations, and monitor system status in real time. User interfaces may be presented on the GUI 102 (FIG. 1, FIG. 12), through a clinician dashboard, or on a mobile caregiver interface, depending on the use case and access level.

### 6.1 GUI (GUI) on Base Unit

The GUI 102 may be implemented as a capacitive or resistive touchscreen mounted directly on the base unit 100, with a layout optimized for real-time use at the bedside or in mobile clinical settings. As shown in FIG. 12, the GUI may include:
Live bar graphs or gauges for RSBI, RR, MV, I:E ratio, and tidal volume;
Color-coded indicators based on severity thresholds and trend behavior;
The ability to compare short-term and long-term therapy trends in single view.
Visual alert symbols, waveform overlays, or parameter dials that reflect current system status;
Navigation controls to switch between signal views, configure thresholds, or acknowledge alerts.

The GUI may also include a capacitive touch button 108 or on-screen equivalent to enable quick user actions, such as pausing monitoring, launching recalibration (FIG. 18), or silencing non-critical alerts.

### 6.2 Role-Based Access and Safety Layers

User interfaces may be configured based on user role (e.g., attending physician, nurse, respiratory therapist, remote caregiver, self user). Each role may have specific access permissions:
Clinicians may approve or reject therapy recommendations (FIG. 20);
Caregivers may receive alerts but not change thresholds;
System administrators may configure reporting or sync schedules.

Safety layers are incorporated to ensure that all therapy-affecting actions are auditable, overrideable, and subject to hard-coded or adaptive threshold review. (see FIG. 25 for dual-mode operation and FIG. 28 for escalation protocols).

Together, these interface components allow seamless interaction between users and the respiratory monitoring system, promoting transparency, trust, and responsiveness. The interface suite supports both real-time care and long-term patient tracking and enables AI-driven systems to remain interpretable and clinician-supervised at every step.

### 7. Closed-Loop and Therapy-Assist Configurations

In various example embodiments, the system may be configured to operate in a closed-loop or therapy-assist mode, allowing it to directly or indirectly influence respiratory therapy delivery based on real-time signal trends and derived parameters. These configurations enable dynamic therapy adaptation to the patient's condition and support claims involving intelligent ventilator integration, safety interlocks, mode switching, and explainable decision-making.

### 7.1 Control Loop Architecture

As shown in FIG. 24, the system may implement a closed-loop architecture that uses respiratory data to compute patient effort metrics and automatically adjust ventilator parameters in response. The control loop may include:
An RSBI computation unit 2420, which calculates the patient's rapid shallow breathing index (RSBI) from respiratory rate and tidal volume inputs derived from MMG or diaphragm displacement data;
A decision threshold logic module 2430, which compares RSBI and other metrics against adaptive thresholds (from for example FIG. 28);
A control signal generator 2440, which formulates a therapy adjustment (e.g., increase inspiratory pressure, decrease backup rate);
A ventilator parameter interface 2450, which communicates with compatible ventilator hardware over a secure bidirectional communication link 2460.

In some cases, the control loop may include feedback from a monitoring feedback loop, which evaluates the patient's post-adjustment response (e.g., RSBI normalization or effort reduction) to confirm or refine the intervention.

### 7.2 Therapy Recommendations and Clinician Overrides

Where automated control is not permitted or desired, the system may instead operate in therapy-assist mode, as shown in FIGS. 20 and 25. In this mode:
Therapy suggestions may be generated by the machine learning inference block 540 and presented in the therapy suggestion panel of the clinician dashboard or mobile device;
Clinicians may review the recommendation in the clinician dashboard or mobile caregiver interface and choose to accept, reject, or modify it;
A therapy override protocol (FIG. 20) may require dual confirmation, signoff, or supervisory review before action is taken;
Safety limits implemented in the safety logic gate 2030 ensure that even clinician-approved changes remain within safe operating parameters. In some configurations, the system includes built-in safety logic gates to verify that any automatically generated ventilator adjustment complies with pre-established safety boundaries. Where confidence scores fall below a defined minimum threshold or where signal quality is degraded, the system may suspend automated control and revert to a monitoring-only mode while notifying a supervising clinician for manual intervention.

All override actions, acceptances, and dismissals may be logged in the system's internal audit trail, and used to further personalize future inferences or threshold tuning (FIG. 28).

### 7.3 Mode Switching and Logic Flow

As shown in FIG. 25, the system may support dynamic switching between monitoring-only mode and therapy-assist mode, depending on:
Device capabilities and configured permissions;
Current patient condition or risk score (e.g., severity score from FIG. 28);
Care environment (e.g., ICU vs. home);
Clinician preference or system policy.

A mode transition condition evaluator 2530 (FIG. 25) may assess whether the criteria for mode switch are met, and initiate transition logic with visual alerts or user confirmation prompts.

The system may also support fallback logic for degraded situations (e.g., communication loss with ventilator, data quality drop, user override), during which the system pauses therapy influence and reverts to monitoring-only status.

### 7.4 Multi-Parameter Control and Escalation Logic

In closed-loop or assist modes, the system may evaluate multiple parameters before recommending or applying therapy changes. For example, as shown in FIG. 26, a control loop based on RSBI may be layered with:
Confidence scoring from data fusion models (FIG. 21);
Posture-aware signal normalization;
Alerting logic from severity scoring modules (FIG. 28).

This enables nuanced behavior such as:
Escalating therapy when RSBI is high and SpO₂ is dropping;
Withholding change during transient motion artifacts;
Flagging low-confidence recommendations for review instead of action.

Each therapy suggestion or automated adjustment may be labeled with metadata (e.g., “Model-based escalation: confidence=91%, priority=high”) and visualized for transparency.

### 7.5 Therapy Targets and Parameters

The system may target multiple ventilator or therapy parameters, including but not limited to:
Inspiratory pressure support or flow volume support;
Trigger sensitivity;
Backup rate;
Expiratory delay or cycling thresholds;
Oxygen flow volume or neuromuscular stimulation (in alternate embodiments).

Adjustments may be made incrementally (e.g., ±1 cm H₂O) or proportionally based on severity scoring or previous therapy response, and may be confirmed visually on the GUI 102 or dashboard prior to action.

Together, these configurations support intelligent respiratory therapy management across multiple care environments, enabling real-time optimization, clinician-supervised assistance, or full closed-loop control, depending on system capabilities and user needs. These features fulfill claims directed to machine learning-driven therapy control, real-time risk evaluation, patient-specific feedback loops, and dual-mode safety-aware architecture.

### 8. Remote Monitoring and Cloud Integration

The respiratory monitoring system may be configured to operate in connection with a secure, HIPAA-compliant or GDPR-compliant cloud-based infrastructure. In various example embodiments, the system may upload, synchronize, or distribute patient data, model inferences, trend reports, alerts, and configuration metadata to remote platforms, enabling real-time clinical oversight, caregiver collaboration, longitudinal analytics, and model adaptation across diverse care environments.

### 8.1 Cloud Architecture and Data Routing

As shown in FIG. 19, remote monitoring may be enabled through a cloud-based infrastructure 1900 that receives physiological signal streams, derived parameters, and inference results from the base unit 100 via the cloud interface. The system may operate continuously or intermittently, using push-based, event-triggered, or scheduled data uploads, depending on configuration.

A remote data routing node 1910 may handle load balancing, endpoint matching, or anonymization. This may support:
Real-time dashboard updates;
Mobile notifications to caregiver devices;
Synchronization with an electronic health record interface 640 (FIG. 6);
Delayed uploads in store-and-forward configurations (e.g., in low-connectivity home settings).

All data may be encrypted in transit and at rest, and access may be governed by role-based access control policies, biometric login, and audit logging.

### 8.2 Web and Mobile Interfaces

Clinicians, caregivers, or authorized observers may access remote data via:
A browser-based dashboard interface;
A mobile caregiver interface, including signal panels, alert logs, and data annotation tools;
Administrative control panels for device fleet management, firmware updates, or reporting.

These tools may support multi-patient review, cross-patient stratification, or care coordination via shared event annotations and synchronized therapy logs.

### 8.3 Alert Propagation and Notification Systems

Alert events generated by the alert logic module 102 (FIG. 1) may be transmitted via the cloud to trigger push notifications, emails, or platform-specific alerts. Each alert may include:
A summary of the triggering parameter (e.g., RSBI=127);
Timestamp, duration, and escalation tier (e.g., “critical,” “moderate”);
Confidence level or override status (e.g., “auto-dismissed after 30 seconds,” “escalated for clinician review”).

Alerts may be configurable per patient, user role, time of day, or known conditions, and may be linked to trend correlation highlights or feedback tracking from the monitoring feedback loop.

8.4 Integration with EHR and Third-Party Systems

The system may include a standards-compliant electronic health record interface 640 (FIG. 6), capable of formatting and transmitting data in HL7, FHIR, or custom JSON formats. This interface may:
Push summary reports, waveform segments, or time-aligned therapy notes to EHR platforms;
Synchronize medication logs, symptom reports, or decision events back into the patient's longitudinal record;
Retrieve clinician annotations to support adaptive inference via the adaptive logic module 2730 (FIG. 27).

System APIs may also expose integration points for third-party analytics, care coordination platforms, or clinical trial dashboards.

### 8.5 Model Update, Audit, and Federated Learning Support

In certain configurations, cloud integration may also support dynamic model updates, including:
Uploading anonymized patient data to retrain population-level models (see FIG. 7);
Supporting federated learning across distributed edge devices (i.e., learning without raw data sharing).

This infrastructure enables adaptive model performance across evolving patient populations, care settings, and use cases, while retaining local data sovereignty.

All cloud interactions may be version-controlled, time-stamped, and logged in secure audit trails to support compliance, traceability, and device lifecycle documentation.

Together, these remote integration capabilities allow the respiratory monitoring system to function not only as a standalone bedside tool, but also as a distributed, learning-enabled clinical platform capable of delivering real-time insight, collaborative oversight, and continuous improvement across hospital, outpatient, and home care environments.

### 9. Use in Specific Clinical Contexts

The respiratory monitoring system described herein may be deployed across a wide range of clinical use cases and patient populations. In various example embodiments, the system may support acute, chronic, procedural, rehabilitative, and home-based respiratory care. The following subsections describe example use contexts, without limitation, that a person of ordinary skill in the art would recognize as supported by the structure and methods disclosed in this application.

### 9.1 Chronic Obstructive Pulmonary Disease (COPD) Monitoring

Patients with COPD may benefit from long-term respiratory monitoring to detect early signs of exacerbation, progressive fatigue, or changes in response to therapy. The system may:
Track RSBI trends, minute volume, and I:E ratio (FIGS. 13-17);
Detect increased effort or shallow breathing using MMG signal curve;
Deliver early warning alerts via the alert logic module;
Trigger caregiver or clinician notifications through the mobile interface or clinician dashboard;
Allow integration of patient-reported symptoms (e.g., dyspnea, fatigue) via the manual input control;
Provide real-time therapy feedback in home settings, with fallback to monitoring-only mode as needed (FIG. 28).

### 9.2 Post-Surgical Respiratory Monitoring

Following abdominal, thoracic, or cardiac surgery, patients may be at risk of postoperative complications. The system may:
Continuously monitor diaphragmatic effort and tidal volume using the diaphragm sensor element 2220 (FIG. 22);
Track I:E ratio and RSBI for signs of progressive deterioration;
Provide visual feedback to staff or patients using the GUI (FIG. 12);
Interface with ventilators in post-anesthesia or ICU environments using closed-loop logic (FIG. 24);
Support mobility and early ambulation by eliminating the need for fixed monitoring lines.

### 9.4 Neuromuscular Disease or Ventilatory Weakness

Patients with ALS, muscular dystrophy, or spinal cord injury may experience respiratory decline due to ventilatory muscle fatigue. The system may:
Track high-resolution tEAdi waveform 302 and detect diminished diaphragm activity;
Identify rising RSBI or paradoxical breathing events (FIG. 17);
Provide haptic feedback 1810 or visual indicators to prompt breath coaching or therapy initiation (FIG. 18);
Support home-based monitoring with real-time trend display and cloud alerts (FIG. 19);
Enable clinicians to view and validate therapy adjustments or overrides via the therapy override protocol (FIG. 20).

### 9.5 Sleep and Ambulatory Use Cases

In sleep studies, home sleep apnea testing, or ambulatory monitoring scenarios, the system may:
Operate in passive monitoring mode with time-synchronized signal logging;
Detect apneic patterns, irregular breathing, or motion-correlated events using accelerometer 2230 and diaphragm sensor 2220;
Upload signal data to a cloud-based dashboard 1900 (FIG. 19) for clinician review;
Align patient sleep diary inputs with waveform anomalies using the input overlay timeline;
Inform CPAP titration or initiate respiratory therapist follow-up.

### 9.6 Rehabilitation and Weaning Environments

In pulmonary rehab or weaning trials, especially in long-term acute care (LTAC) settings, the system may:
Provide breath-by-breath RSBI feedback during ventilator liberation trials;
Log success/failure markers and integrate clinician judgment into the adaptive logic module 2730 (FIG. 27);
Drive safe escalation of support using escalation decision logic 2830 (FIG. 28);
Provide visual cues to respiratory therapists or patients via the therapy overlay graph;
Integrate with exercise, neuromuscular training, or incentive spirometry programs.

Accordingly, these clinical contexts demonstrate that the system is not limited to a single use case, device configuration, or environment, but instead enables a comprehensive, intelligent respiratory monitoring solution that may be tailored to the patient, care setting, and therapeutic objectives-supporting claims directed to flexibility, adaptability, safety, and clinical decision support.

### 10.0 End-to-End Example Use Scenario

In one illustrative example, a 72-year-old patient recovering from cardiac surgery is admitted to a step-down telemetry unit. To support early detection of respiratory decline, a nurse attaches the system's wearable diaphragm sensor 200, positioning the sensor housing 204 on the upper abdomen and securing it with the belt 210.

The patient's real-time RSBI, tidal volume, and respiratory rate are computed locally via the processor/GUI 102 and visualized on the GUI shown in FIG. 12. A trend line begins forming on the dashboard interface, shared with the respiratory therapist and the remote ICU oversight team. When the patient shifts from supine to seated, the accelerometer detects the change and triggers posture-aware threshold recalibration using the logic described above.

During hour 3 of monitoring, the system detects a rising RSBI and falling SpO₂. The adaptive logic references the patient's surgical history and recent weaning trends to adjust alert thresholds downward. The alert logic module raises a moderate-severity alert, which is logged and shown both on the base unit GUI and pushed to the mobile caregiver interface.

The clinician accesses the dashboard and reviews both the 3D tEAdi signal surface 408 (FIG. 4) and the patient's trend overlay, which includes a manual input note from the caregiver: “Patient appears fatigued. Shallow breathing observed.” Based on this corroborating evidence, the system generates a therapy suggestion recommending increased inspiratory pressure support.

Because the ventilator is connected via the ventilator parameter interface, the clinician elects to approve the suggestion, which is executed by the control signal generator 2440. The system logs the change, updates thresholds, and resumes real-time feedback. Within 20 minutes, the patient's RSBI drops below the adapted target value, and the system logs a recovery event. Trend data is automatically stored and synchronized with the cloud-based infrastructure 1900 (FIG. 19), where it may be used for population-level model retraining.

This scenario illustrates how the system combines wearable sensing, real-time AI inference, adaptive decision thresholds, clinician input, and therapy integration to support early intervention, improve patient outcomes, and reduce cognitive burden on the care team. The system remains flexible enough to operate passively in home settings, aggressively in closed-loop ICU environments, or in a hybrid assist mode with optional clinician override, or as a standalone monitoring solution post-ventilatory support, depending on configuration.

### 11.0 Additional Hardware & Software Implementation Details

Although an example processing system has been described above, implementations of the subject matter and the functional operations described herein can be implemented in other types of digital electronic circuitry, or in computer software, firmware, or hardware, including the structures disclosed in this specification and their structural equivalents, or in combinations of one or more of them.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described herein can be implemented as one or more computer programs, i.e., one or more modules of computer program instructions, encoded on computer storage medium for execution by, or to control the operation of, information/data processing apparatus. Alternatively, or in addition, the program instructions can be encoded on an artificially generated propagated signal, e.g., a machine-generated electrical, optical, or electromagnetic signal, which is generated to encode information/data for transmission to suitable receiver apparatus for execution by an information/data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in. one or more separate physical components or media (e.g., multiple CDs, disks, or other storage devices).

The operations described herein can be implemented as operations performed by an information/data processing apparatus on information/data stored on one or more computer-readable storage devices or received from other sources.

The terms “‘processor”, “computer.” “data processing apparatus”, and the like encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing, and grid computing infrastructures.

A computer program (also known as a program, software, software application, script, code, program code, and the like) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or information/data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described herein can be performed by one or more programmable processors executing one or more computer programs to perform actions by operating on input information/data and generating output. Processors suitable for the execution of a computer program include, by way of example, both general and special purpose microprocessors, and any one or more processors of any kind of digital computer. Generally, a processor will receive instructions and information/data from a read only memory′ or a random-access memory or both. The essential elements of a computer are a processor for performing actions in accordance with instructions and one or more memory devices for storing instructions and data. Generally, a computer will also include, or be operatively coupled to receive information/data from or transfer information/data to, or both, one or more mass storage devices for storing data, e.g., magnetic, magneto-optical disks, or optical disks. However, a computer need not have such devices. Devices suitable for storing computer program instructions and information/data include all forms of non-volatile memory, media, and memory′ devices, including by way of example semiconductor memory devices, e.g., EPROM, EEPROM, and flash memory devices; magnetic disks, e.g., internal hard disks or removable disks; magneto-optical disks; and CD ROM and DVD-ROM disks. The processor and the memory can be supplemented by, or incorporated in, special purpose logic circuitry′.

To provide for interaction with a user, embodiments of the subject matter described herein can be implemented on a computer having a display device, e.g., a CRT (cathode ray tube) or LCD (liquid crystal display) monitor, for displaying information/data to the user and a keyboard and a pointing device, e.g., a mouse or a trackball, by which the user can provide input to the computer. Other kinds of devices can be used to provide for interaction with a user as well; for example, feedback provided to the user can be any form of sensor feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described herein can be implemented in a computing system that includes a backend component, e.g., as an information/data server, or that includes a middleware component, e.g., an application server, or that includes a frontend component, e.g., a client computer having a GUI or a web browser through which a user can interact with an implementation of the subject matter described herein, or any combination of one or more such back end, middleware, or front end components. The components of the system can be interconnected by any form or medium of digital information/data communication, e.g., a communication network. Examples of communication networks include a local area network (“LAN”) and a wide area network (“WAN”), an inter-network (e g., the Internet), and peer-to-peer networks (e.g., ad hoc peer-to-peer networks).

The computing system can include clients and servers. A client and server are generally remote from each other and typically interact through a communication network, the relationship of client and server arises by virtue of computer programs running on the respective computers and having a client-server relationship with each other. In some embodiments, a server transmits information/data (e.g., an HTML page) to a client device (e.g., for purposes of displaying information/data to and receiving user input from a user interacting with the client device). Information/data generated at the client device (e.g., a result of the user interaction) can be received from the client device at the server.

While this specification contains many specific implementation details, these should not be construed as limitations on the scope of any embodiment or of what may be claimed, but rather as descriptions of features specific to particular embodiments. Certain features that are described herein in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations arc depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

In some embodiments of the system described herein, the entire system can be implemented and offered to the end-users and operators over the Internet, in a so-called cloud implementation. No local installation of software or hardware would be needed, and the end-users and operators would be allowed access to the systems of the system described herein directly over the Internet, using either a web browser or similar software on a client, which client could be a desktop, laptop, mobile device, and so on. This eliminates any need for custom software installation on the client side and increases the flexibility of delivery of the service (software-as-a-service), and increases user satisfaction and ease of use. Various business models, revenue models, and delivery mechanisms for the system described herein are envisioned, and are all to be considered within the scope of the system described herein.

In general, the method executed to implement the embodiments of the invention, may be implemented as part of an operating system or a specific application, component, program, object, module, or sequence of instructions referred to as “program code,” “computer program(s)”, “computer code(s).” and the like. The computer programs typically comprise one or more instructions set at various times in various memory and storage devices in a computer, and that, when read and executed by one or more processors in a computer, cause the computer to perform operations necessary to execute elements involving the various aspects of the invention. Moreover, while the invention has been described in the context of fully functioning computers and computer systems, those skilled in the art will appreciate that the various embodiments of the invention are capable of being distributed as a program product in a variety of forms, and that the invention applies equally regardless of the particular type of machine or computer-readable media used to actually affect the distribution. Examples of computer-readable media include but are not limited to recordable type media such as volatile and non-volatile (or non-transitory) memory devices, floppy and other removable disks, hard disk drives, optical disks, which include Compact Disk Read-Only Memory (CD ROMS), Digital Versatile Disks (DVDs), etc., as well as digital and analog communication media.

One of ordinary skill in the art knows that the use cases, structures, schematics, flow diagrams, and steps may be performed in any order or sub-combination, while the inventive concept of the system described herein remains without departing from the broader scope of the invention. Every embodiment may be unique, and step(s) of method(s) may be either shortened or lengthened, overlapped with other activities, postponed, delayed, and/or continued after a time gap, such that every active user and running application program is accommodated by the server(s) to practice the methods of the system described herein.

For simplicity of explanation, the embodiments of the methods of this disclosure are depicted and described as a series of acts or steps. However, acts or steps in accordance with this disclosure can occur in various orders and/or concurrently, and with other acts or steps not presented and described herein. Furthermore, not all illustrated acts or steps may be required to implement the methods in accordance with the disclosed subject matter. In addition, those skilled in the art will understand and appreciate that the methods could alternatively be represented as a series of interrelated states via a state diagram or events or their equivalent.

As used herein, the singular forms “′a,” “″an,” and “′the” include plural references unless the context clearly indicates otherwise. Thus, for example, reference to “a cable” includes a single cable as well as a bundle of two or more different cables, and the like.

Tire terms “comprise,” “comprising,” “includes,” “including,” “have,” “having,” and the like, used in the specification and claims are meant to be open-ended and not restrictive, meaning “including but not limited to.”

In the foregoing description, numerous specific details are set forth, such as specific structures, dimensions, processes, parameters, etc., to provide a thorough understanding of the system described herein. The particular features, structures, materials, or characteristics may be combined in any suitable manner in one or more embodiments. The words “example”, “exemplary”, “illustrative” and the like, are used herein to mean serving as an example, instance, or illustration. Any aspect or design described herein as “example” or its equivalents is not necessarily to be construed as preferred or advantageous over other aspects or designs. Rather, use of the words “example” or equivalents is intended to present concepts in a concrete fashion.

As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A, X includes B, or X includes both A and B, then “X includes A or B” is satisfied under any of the foregoing instances.

Reference throughout this specification to “an embodiment,” “certain embodiments,” or “one embodiment” means that a particular feature, structure, or characteristic described in connection with the embodiment is included in at least one embodiment. Thus, the appearances of the phrase “an embodiment,” “certain embodiments,” or “one embodiment” throughout this specification are not necessarily all referring to the same embodiment.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. For example, in some exemplary embodiments, the term “about” may include the recited number ±10%, such that “about 10” would include from 9 to 11. In other exemplary embodiments, the term “about” may include the recited number ±X %, where X is considered the normal variation in said measurement by one of ordinary skill in the art.

Although the system may be described in functional terms (e.g., ‘configured to . . . ’), such descriptions are not intended to invoke 35 U.S.C. § 112(f) unless explicitly stated as ‘means for.’

Features which are described in the context of separate embodiments may also be provided in combination in a single embodiment. Conversely, various features which are, for brevity, described in the context of a single embodiment, may also be provided separately or in any suitable sub-combination, the applicant hereby gives notice that new claims may be formulated to such features and/or combinations of such features during the prosecution of the present application or of any further application derived therefrom. Features of the transitory physical storage medium described may be incorporated into/used in a corresponding method, digital documentation system and/or system, and vice versa. Accordingly, it should be understood that the structural and functional components described herein may be rearranged, combined, or separated to form alternative embodiments, including method-based, apparatus-based, and computer-implemented configurations. Unless otherwise noted, any claim element described as a hardware component may alternatively be implemented as software, and vice versa, to the extent technically feasible. For example, in various example embodiments, certain system components may be implemented entirely in software, including sensor emulation, retrospective data analysis, or inference replay. The core signal processing and decision support logic may be hosted on tablets, smartphones, or cloud platforms without requiring a physical base unit, thereby enabling deployment in low-resource settings or for retrospective clinical research. Likewise, in various implementations, the respiratory analysis algorithms, machine learning models, and control logic described herein may be embodied as software instructions stored on a non-transitory computer-readable medium and executed by one or more processors. The code may include embedded firmware, containerized services, or downloadable mobile applications, and may operate on local processors within the base unit or wearable sensor, or remotely via cloud infrastructure.

### 12.0 Example Embodiments

Provided hereafter are non-limiting examples of certain embodiments of the technology.

A1. A wearable patient monitoring system comprising:
a diaphragm-mounted flexible capacitive sensor configured to measure mechanomyographic data;
a photoplethysmographic (PPG) sensor configured to measure oxygen saturation (SpO₂);
a carbon dioxide (CO₂) sensor and a temperature sensor;
a processor configured to create synchronized multi-sensor signals by receiving and synchronizing signals from the diaphragm sensor, PPG sensor, CO₂ sensor, and temperature sensor;
a machine learning model trained to derive respiratory health indicators based on the synchronized multi-sensor signals;
a user interface configured to display the health indicators to a clinician in real time.

A1.1. The system of embodiment A1, wherein the machine learning model is trained using datasets labeled with specific respiratory conditions, including COPD, respiratory fatigue, and ventilation-perfusion mismatch.

A1.2. The system of embodiment A1, wherein the processor is configured to identify a correlation trend over time between changes in diaphragmatic effort and changes in oxygen saturation, and to associate the trend with a respiratory condition selected from the group consisting of respiratory fatigue, neuromuscular decline, or ventilation-perfusion mismatch.

A1.3. The system of embodiment A1, wherein the system is configured to calculate a severity score based on aggregated sensor trends to prioritize clinical alerts.

A1.4. The system of embodiment A1, wherein the GUI is configured to display a real-time active multi-parameter dashboard including SpO₂, respiratory rate, and minute volume values.

A1.5. The system of embodiment A1, wherein the system is configured to use data from the temperature sensor to distinguish fever-induced hyperventilation from other respiratory changes.

A1.6 The system of embodiment A1, wherein the system is configured to use data from the CO₂ sensor to track end-tidal or partial carbon dioxide levels for ventilatory assessment.

A2. A method of operating a respiratory monitoring system, comprising:
acquiring patient posture data from a body-position sensor;
receiving patient-reported symptom input via a user interface;
collecting physiological data from a diaphragm sensor and a PPG sensor;
selecting one of a plurality of machine learning inference profiles based at least in part on the posture data and symptom input;
applying the selected profile to derive a respiratory parameter comprising at least one of respiratory rate or minute volume; and
generating an alert when the respiratory parameter deviates from a profile-specific context threshold.

A2.1. The method of embodiment A2, further comprising applying real-time filtering to account for movement artifacts based on position data.

A2.2. The method of embodiment A2, wherein the patient posture data corresponds to one or more patient positions including at least one of: prone, supine, sitting, inclined, or walking.

A2.3. The method of embodiment A2, wherein the selected inference profile adjusts alert thresholds for respiratory parameters.

A2.4. The method of embodiment A2, wherein the alert includes a recommendation for therapy escalation or de-escalation.

A2.5. The method of embodiment A2, wherein the patient-reported symptom input comprises at least one of perceived shortness of breath, pain level, or fatigue.

A2.6. The method of embodiment A2, further comprising comparing derived respiratory parameters against a patient-specific baseline to detect respiratory deviations associated with clinical events.

A2.7. The method of embodiment A2, further comprising comparing the sensitivity of signals from a diaphragm sensor and a PPG sensor in relation to changes in therapy mode or settings, or through a longitudinal comparison of the sensors' sensitivity responses over time with respect to each other.

A2.8. The method of embodiment A2, further comprising identifying patterns associated with respiratory distress caused by airway inflammation, internal bleeding, or fluid overload by further processing the derived respiratory parameters.

A2.9. The method of embodiment A2, wherein the system operates in one of two modes: a therapy-assist mode that adjusts therapy parameters based on sensor feedback, and a monitoring-only mode that tracks sensor data.

A2.10. The method of embodiment A2, further comprising storing patient-reported symptom data and derived parameters and conducting longitudinal trend analysis using the stored patient-reported symptom data and derived parameters.

A2.11. The method of embodiment A2, further comprising:
dynamically selecting an inference model and adjusting respiratory alert thresholds based on a combination of:
   a. real-time patient posture data acquired from a body-position sensor;
   b. patient-reported symptom input received through a user interface; and
   c. movement artifacts identified during signal preprocessing.

A3. A cloud-integrated respiratory monitoring system comprising:
a wearable respiratory sensor unit comprising a diaphragm sensor and a PPG sensor;
a processor configured to derive respiratory parameters from the sensor signals;
a cloud interface module configured to transmit derived parameters and time-stamped events to a remote server;
a GUI hosted on the remote server, the interface configured to display patient-specific respiratory trends along with user-entered treatment notes;
an alert engine configured to transmit prioritized notifications to remote caregivers based on deviations in the respiratory trends.

A3.1. The system of embodiment A3, wherein the cloud interface includes HIPAA-compliant data encryption and access controls.

A3.2. The system of embodiment A3, wherein the GUI is configured to display real-time updates across a plurality of patients simultaneously.

A3.3. The system of embodiment A3, the system is configured to time-stamp treatment notes entered by a clinician and correlate the treatment notes with sensor data trends.

A3.4. The system of embodiment A3, wherein the alert engine is configured to rank notifications by a calculated severity index.

A3.5. The system of embodiment A3, wherein the remote server is configured to export respiratory parameter logs to an electronic health record (EHR) system.

A.4. A method of monitoring a patient following surgery, comprising:
positioning a wearable sensor comprising a diaphragm sensor and an accelerometer over the patient's diaphragm;
acquiring respiratory signals during a post-operative recovery period;
deriving respiratory parameters comprising at least respiratory rate and minute volume;
detecting changes in the respiratory parameters indicative of potential complications selected from the group consisting of internal bleeding, fluid retention, and respiratory deterioration; and
generating an alert or flagging a patient record for clinician review.

A5. A respiratory support system comprising:
a wearable respiratory sensor configured to measure diaphragm movement and oxygen saturation;
a processor configured to derive respiratory parameters including at least respiratory rate and minute volume from sensor signals;
a ventilator controller operatively coupled to a respiratory therapy device;
wherein the ventilator controller is configured to adjust one or more therapy parameters selected from pressure, flow rate, or oxygen concentration in response to changes in the derived respiratory parameters.

A5.1. The system of embodiment A5, wherein the respiratory sensor comprises both a diaphragm sensor and a PPG sensor.

A5.2. The system of embodiment A5, wherein the ventilator controller is configured to operate automatically without clinician input.

A5.3. The system of embodiment A5, wherein the processor is configured to calculate a rapid shallow breathing index (RSBI) and to adjust inspiratory pressure accordingly.

A5.4. The system of embodiment A5, wherein the system is configured to allow therapy adjustments to be confirmed by clinician override.

A6. A patient-managed respiratory monitoring system comprising:
a wearable diaphragm sensor configured to detect mechanomyographic activity;
a mobile device application configured to:
   receive sensor data wirelessly;
   display real-time respiratory trends to the patient;
   receive patient-reported inputs including symptoms or perceived distress; and
   transmit the data to a remote clinical dashboard.

A6.1. The system of embodiment A6, wherein the mobile application includes voice-input fields for symptom reporting.

A6.2. The system of embodiment A6, wherein the mobile device application is configured to store historical respiratory data and to transmit weekly summaries to a clinical provider.

A6.3. The system of embodiment A6, wherein the wearable sensor includes a haptic feedback element configured to alert the patient.

A6.4. The system of embodiment A6, wherein the system is configured to display alerts on both the mobile device and a remote clinical dashboard in synchrony.

A6.5. The system of embodiment A6, wherein the mobile application is configured to enable entry of therapy changes or medication updates and to display those alongside physiological parameter trends.

A7. A respiratory monitoring system comprising:
a diaphragm-mounted sensor configured to detect time-varying mechanomyographic (MMG) data corresponding to diaphragmatic effort;
a photoplethysmographic (PPG) sensor configured to detect oxygen saturation (SpO₂);
a processor configured to determine a time-correlated divergence between the diaphragmatic effort signal and the SpO₂ signal;
an inference engine configured to identify a respiratory abnormality indicative of post-dialysis fatigue or impaired gas exchange based on the divergence; and
an alert module configured to generate a notification in response to detection of the respiratory abnormality.

A7.1. The system of embodiment A7, wherein the alert module is configured to transmit the notification to a caregiver via a mobile application or web-based interface or both.

A7.2. The system of embodiment A7, wherein the processor and sensors are configured for continuous operation during an interdialytic period.

A7.3. The system of embodiment A7, wherein the inference engine comprises a rule-based threshold module and a neural network classifier configured to validate detection of the respiratory abnormality.

A7.4. The system of embodiment A7, wherein the inference engine is configured to distinguish between diaphragm fatigue and central hypoventilation based on a time-correlated divergence between diaphragmatic effort signals and oxygen saturation (SpO₂) signals.

A8. A respiratory analytics platform comprising:
a server configured to receive multi-parameter respiratory data from a plurality of wearable monitoring systems;
a machine learning model trained using population-level datasets labeled with respiratory outcomes to generate predictive thresholds;
a patient-specific adaptation module configured to adjust alert sensitivity based on individualized deviation from the population-trained thresholds; and
a notification engine configured to transmit alerts to clinicians when patient-specific adjusted thresholds are exceeded.

A8.1. The platform of embodiment A8, wherein the population-trained model includes clinical outcome labels including hospitalization or COPD exacerbation or both.

A8.2. The platform of embodiment A8, wherein the patient-specific alert threshold is configured to be recalculated weekly or more frequently based on trend data.

A8.3. The platform of embodiment A8, wherein the platform is configured to be integrated with a hospital's electronic health record system for real-time decision support.

A8.4. The platform of embodiment A8, wherein the machine learning engine is configured to be calibrated with data from at least 100 patients.

A8.5. The platform of embodiment A8, wherein the machine learning engine is configured to compare patient respiratory patterns against condition-specific baselines to identify post-operative complications.

A8.6. The platform of embodiment A8, wherein the machine learning engine is configured to compare patient respiratory patterns against condition-specific baselines to identify overdue dialysis events.

A8.7. The platform of embodiment A8, wherein the machine learning engine is configured to compare patient respiratory patterns against condition-specific baselines to identify changes in patient condition during dialysis procedure (peritoneal or hemodialysis).

Although the system described herein has been described with reference to specific exemplary embodiments, it will be evident that the various modifications and changes can be made to these embodiments without departing from the broader scope of the invention. Accordingly, the specification and drawings are to be regarded in an illustrative sense rather than in a restrictive sense. It will also be apparent to the skilled artisan that the embodiments described above are specific examples of a single broader invention which may have greater scope than any of the singular descriptions taught. There may be many alterations made in the descriptions without departing from the scope of the present invention, which is defined by the claims.

1. A respiratory monitoring system comprising:
a wearable sensor configured to be positioned over a patient's diaphragm and comprising:
a flexible capacitive diaphragm sensor configured to detect mechanomyographic (MMG) data in three spatial dimensions; and
an accelerometer configured to detect patient motion;
a processor configured to receive signals comprising time-varying signals from the diaphragm sensor and accelerometer;
a machine learning module stored in memory and configured to be executed by the processor, the machine learning module trained to derive, based on the received signals, one or more respiratory parameters selected from the group consisting of respiratory rate, inspiratory and expiratory phases, inspiratory-expiratory ratio, tidal volume, minute volume, respiratory mechanics, rapid shallow breathing index, cough detection, and dyspnea detection;
a GUI configured to display at least one of said respiratory parameters in real time; and
an alert module configured to generate a notification when one or more of the respiratory parameters deviates from a predefined threshold.

2. The system of claim 1, further comprising a non-elastic adjustable belt and buckle configured to secure the wearable sensor to the patient.

3. The system of claim 1, wherein the diaphragm sensor comprises two or more stretchable electrodes separated by a dielectric polymer.

4. The system of claim 1, wherein the accelerometer and diaphragm sensor are enclosed in a housing configured for patient comfort during long-term use.

5. The system of claim 1, wherein the machine learning module is a deep learning model comprising at least three hidden layers.

6. The system of claim 1, wherein the processor is configured to derive both inspiratory and expiratory tidal volume from the signals.

7. The system of claim 1, wherein the machine learning module is configured to classify patient condition based on one or more of the respiratory parameters, the condition selected from the group consisting of diaphragm atrophy, neuromuscular weakness, or restrictive lung disease.

8. The method of claim 1, wherein the machine learning model comprises training data obtained from both healthy individuals and individuals with chronic obstructive pulmonary disease.

9. The system of claim 1, wherein the GUI is configured to display real-time active bar graphs corresponding to one or more respiratory parameter's current, minimum, maximum, and average values over a selected time window.

10. The system of claim 1, wherein the alert module includes a 360-degree visual indicator configured to be visible from all lateral directions.

11. The system of claim 1, wherein the alert module is configured to prioritize alerts based on a severity index computed from multiple parameters.

12. The system of claim 1, further comprising a wireless communication module and a cloud database interface, the system being configured to continuously monitor patient respiratory parameters remotely in home care or outpatient settings, and to transmit time-stamped respiratory data and alerts to remote caregivers or clinicians.

13. The system of claim 1, wherein the machine learning module is configured to identify patterns indicative of early-stage COPD exacerbation based on multi-parameter trend analysis of historical respiratory training data.

14. The system of claim 1, wherein the machine learning module is configured to detect deviations indicative of clinical deterioration by comparing current respiratory parameter values with patient-specific baselines.

15. The system of claim 1, further comprising a thoracic impedance sensor configured to provide impedance-based respiratory measurements as part of multi-modal parameter analysis.

16. The system of claim 1, wherein the machine learning module is configured to adapt its classification thresholds based on an individual patient's historical data, physiological characteristics, and longitudinal respiratory trends.

17. The system of claim 1, wherein the wearable sensor is configured for use during patient recovery following major surgery selected from the group consisting of liver surgery, cardiac surgery, and abdominal procedures, and wherein the system is configured to detect changes in respiratory patterns indicative of post-operative complications.

18. The system of claim 1, wherein the processor is configured to generate visualizations correlating patient-entered treatment changes with measured respiratory parameters in a trend display.

19. The system of claim 1, wherein the processor is configured to generate a 3D surface profile of diaphragmatic activity using three-dimensional tEAdi signal data.

20. A system for monitoring respiratory parameters, comprising:
a wearable respiratory sensor configured to be positioned over a patient's diaphragm, the sensor including a flexible capacitive element and an accelerometer;
a processor configured to extract time-varying signals from the sensor;
a machine learning engine trained to derive multiple respiratory parameters from the signals, including respiratory rate and minute volume;
a user interface configured to display trends in said parameters over time;
a therapy integration module configured to:
(i) receive user input regarding changes in respiratory therapy, and
(ii) correlate said input with parameter trends to facilitate adjustment of therapy settings;
wherein the system is operable in both a therapy-assist mode, in which the system provides feedback for adjusting therapy settings, and a post-therapy monitoring mode, in which the system tracks and displays long-term respiratory trends without active therapy integration.

21. The system of claim 20, wherein the therapy integration module is configured to transmit respiratory parameter trends or alert conditions as control signals to a connected respiratory therapy device.

22. The system of claim 20, wherein the user interface is configured to receive manual inputs of changes to therapy, medication, or patient status, and to display said changes alongside respiratory trends, and wherein such inputs are configured to be stored in association with time-stamped parameter records for correlation and review.

23. The system of claim 20, wherein the processor is configured to optimize therapy settings by correlating changes in tidal volume and respiratory rate with clinical interventions.

24. The system of claim 20, wherein the post-therapy monitoring mode is configured to analyze long-term trends of respiratory parameters stored in a local or cloud-based database.

25. The system of claim 20, wherein the system is configured to automatically switch between therapy-assist mode and monitoring mode based on detection of a change in respiratory support device status.

26. A method of monitoring a patient's respiratory condition, comprising:
detecting diaphragmatic movement using a wearable sensor comprising a flexible capacitive sensor and an accelerometer positioned over the patient's diaphragm;
generating a time-varying signal representing three-dimensional mechanical motion of the diaphragm;
processing the signal using a trained machine learning model to derive a set of respiratory parameters comprising one or more of respiratory rate and tidal volume;
comparing at least one of the respiratory parameters to a predefined threshold; and
generating an alert when the threshold is exceeded.
